(19) United States
(12) Patent Application Publication
(10) Pub. No.: US 2016/0003808 A1
(43) Pub. Date: Jan. 7, 2016

(54) MOLECULAR TARGETS AND COMPOUNDS, AND METHODS TO IDENTIFY THE SAME, USEFUL IN THE TREATMENT OF DISEASES ASSOCIATED WITH EPITHELIAL MESENCHYMAL TRANSITION

(71) Applicant: GALAPAGOS NV, Mechelen (BE)

(72) Inventors: Richard Antonius Jozef Janssen, Leiden (NL); Annemarie Nicolete Lekkerkerker, Palo Alto, CA (US); Jamil Aarbiou, Leiden (NL)

(21) Appl. No.: 14/775,861

(22) PCT Filed: Mar. 7, 2014
(86) PCT No.: PCT/EP2014/054443

§ 371 Date: Sep. 14, 2015

Related U.S. Application Data
(60) Provisional application No. 61/781,795, filed on Mar. 14, 2013.

(51) Int. Cl. G01N 33/50; C07K 16/40; C07K 16/18; C12N 15/113

(52) U.S. Cl. CPC G01N 33/5073; C12N 15/113; C07K 16/40; C07K 16/18; G01N 2333/96486; G01N 2333/78; G01N 2333/70596; G01N 2500/10; G01N 2500/04; C12N 2310/531; C12N 2320/30; C12N 2310/111; C12N 2310/14; C12N 2310/141; C12N 2310/12

(57) ABSTRACT

The present invention relates to methods and assays for identifying agents useful in the treatment of diseases associated with epithelial mesenchymal transition (EMT), in particular fibrotic diseases and cancer. The invention provides polypeptide and nucleic acid TARGETs, siRNA sequences based on these TARGETs and antibodies against the TARGETs. The invention is further related to pharmaceutical composition comprising siRNA sequences based on the TARGETs and antibodies against the TARGETs for use in the treatment of diseases associated with epithelial mesenchymal transition, in particular fibrotic disease and cancer. The invention further provides in vitro methods for inhibition of epithelial mesenchymal transition.

### TECHNICAL FIELD OF THE INVENTION

The present invention is in the field of molecular biology and biochemistry. The present invention relates to methods for identifying agents useful in treatment of fibrotic disease, in particular, agents that inhibit epithelial mesenchymal transition (EMT) Inhibition of EMT is useful in the prevention and/or treatment of diseases where EMT plays an important role. In particular, the present invention provides methods for identifying agents for use in the prevention and/or treatment of fibrotic diseases and cancer.

### BACKGROUND OF THE INVENTION

The epithelial mesenchymal transition (EMT) is the process during which epithelial cells convert into mesenchymal cells. Generally, such process is reversible and is characterized by changes in cell adhesion and cellular mobility. This process is commonly accompanied by repression of the expression of E-cadherin, and the generated mesenchymal cells are characterized by new migratory, invasive and fibrogenic properties. EMT is an important biological process and plays an important role in embryogenesis and normal wound healing (Hay, 2005). Although, EMT contributes to tissue repair, it can also adversely cause organ fibrosis and promote carcinoma progression through a variety of mechanisms. EMT was shown to play role in cancer progression and metastasis (Thiery, 2002). EMT has also been identified to contribute to the pathogenesis of degenerative fibrotic disorders in different organs, including the lung (Wilson, 2009; Lekkerkerker et al, 2012).

Hepatocytes and biliary epithelial cells in the liver (Firrincieli et al, 2010; Choi et al, 2009) and epithelial cells in the lung may contribute to fibrosis through EMT. These epithelial cells lose their epithelial phenotype, acquire fibroblast-like properties, and display reduced cell adhesion and increased motility. During this process, epithelial cells lose their cellular polarity and undergo remodeling of epithelial cell-cell and cell-matrix adhesion contacts. The reduction of adhesion molecules allows the cells to detach from the epithelial layer and migrate towards the site of injury or inflammation where they demonstrate their profibrotic effects. During EMT typical markers of polarized epithelial cells, such as E-cadherin and some cytokeratins, are lost, whereas markers of mesenchymal cells such as vimentin, and N-cadherin or markers of myofibroblasts, such as a-smooth muscle actin (α-SMA), are acquired (Zavadil et al, 2005). Several studies have demonstrated that EMT may occur in human lung epithelial cell lines and primary bronchial epithelial cells upon exposure to TGFβ (Câmara, 2010; Kasai, 2005). The precise mechanisms of this process still need to be explored. It is known that TGFβ is elicited predominantly by activated inflammatory cells including macrophages that are attracted to the site of injury and binds the TGFβ type 2 receptor (TGFBR2), which then forms a complex with TGFβ type 1 receptor (TGFBR1). This complex initiates a signaling cascade in which a complex of Smad2 and Smad3 and subsequently Smad4 is activated. The activated complex of Smads translocates to the nucleus and induces gene transcription. Although TGFβ appears to be essential for EMT, other factors may influence this process. Inflammatory cytokines, such as IL-1β and TNFα (Borthwick et al, 2010; Camara et al, 2010), and also bacteria (Borthwick et al, 2011) and viruses (Shimamura et al, 2010) were shown to enhance TGFβ-induced markers of EMT, even though the cytokines themselves do not have an EMT inducing capacity. The precise mechanisms of this process still need to be studied further.

Some examples of modulation of EMT utilize lipocalin 2 (WO2006/078717) or regulators of GAPR-1 protein (WO2007/038264). WO2007/069839 discloses use of Erythropoietin (EPO) for the preparation of an agent for inhibition of the EMT. It further describes a method for prevention and treatment of fibrosis using EPO.

US2006/234911 discloses pharmaceutical compositions comprising a kinase inhibitor capable of reversing EMT. Selected disclosed inhibitors are the inhibitors of TGFβ, RhoA or p38 MAP kinases. Similarly, the invention describes a method of reversing EMT in a patient suffering from fibrosis or cancer.

Known targets and inhibitors of those targets still possess many challenges. For example, there are many processes regulated by TGFβ and the use of inhibitors against TGFβ also affects cellular processes essential for normal cell function. Therefore, such inhibitors provoke several secondary effects in patients suffering from cancer or fibrotic conditions. Therefore, further understanding of the EMT is needed to develop more efficient methods to identify new drug targets and therapies.

In the past decades much effort has been put into the development of in vitro and in vivo models to unravel the molecular mechanisms regulating EMT processes in the lung. Many studies focused on various cell lines derived from the lung (e.g. A549, NCI-H292, BEAS2B and 16HBE) as an in vitro model for molecular and cellular processes in lung epithelium and these have contributed considerably to the present understanding of the signaling pathways epithelial cells utilize to exercise their effects. However, cell lines may not always provide the best model for studying molecular processes as they often carry transforming mutations and have abnormal chromosome copy numbers. In addition, extensive passaging of cells and varying culture conditions may introduce additional genetic and post-transcriptional changes affecting molecular and cellular function and causing inconsistencies between different reports.

The use of cell lines may therefore introduce biases towards certain molecular pathways or the risk that important cellular processes are overlooked. Employment of primary cells and preferably those from patients will minimize such risks and provide us with better insights in the molecular processes involved in the EMT.

Finally, better and more relevant in vitro models of EMT are needed. It would be advantageous to set up more functional cellular assays employing patient-derived cells in physiological relevant conditions. Such assays could then be used to perform functional genomics studies to identify novel drug targets, and new compounds for the treatment of diseases associated with EMT, in particular fibrosis and carcinomas.

### SUMMARY OF THE INVENTION

The present invention is based on the discovery that agents that inhibit the expression and/or activity of the TARGETS disclosed herein are capable of inhibiting epithelial mesenchymal transition (EMT), as indicated by a inhibition of expression or/and release of markers of EMT. In particular, the suppression of the release or expression of MMP10, fibronectin, E-cadherin and/or soluble fibronectin are exemplary indicators. The present invention, therefore, provides TARGETS which play a role in EMT, methods for screening for agents capable of down-regulating the expression and/or activity of TARGETS and the use of these agents in the prevention and/or treatment of diseases associated with EMT, in particular fibrosis and carcinomas. The present invention provides TARGETS which are involved in the biology of EMT, in particular with fibrotic disorders associated with epithelial mesenchymal transition. In a particular aspect, the present invention provides TARGETS which are involved in or otherwise associated with development of fibrotic diseases and cancer.

The present invention relates to a method for identifying a compound useful for the treatment of a disease associated with EMT, said method comprising: contacting a test compound with a TARGET polypeptide, fragments and structurally functional derivatives thereof, determining a binding affinity of the test compound to said polypeptide or an activity of said polypeptide, contacting the test compound with a population of epithelial cells, measuring a property related to EMT, and identifying a compound inhibiting EMT and which either demonstrates a binding affinity to said polypeptide or is able to inhibit the activity of said polypeptide.

The present invention further relates to a method for identifying a compound useful for the treatment of a disease associated with EMT, said method comprising: contacting a test compound with population of epithelial cells and expressing a TARGET polypeptide, measuring expression and/or amount of said polypeptide in said cells, measuring a property related to EMT, and identifying a compound which reduces the expression and/or amount of said polypeptide and which is inhibiting EMT.

The present invention relates to a method for identifying a compound inhibiting EMT said method comprising: contacting a test compound with a TARGET polypeptide, fragments or structurally functional derivatives thereof, determining a binding affinity of the test compound to said polypeptide or an activity of said polypeptide, contacting the test compound with a population of epithelial cells, measuring a property related to EMT, and identifying a compound inhibiting EMT and which demonstrates a binding affinity to said polypeptide and/or is able to inhibit the activity of said polypeptide.

The present invention provides a method for identifying a compound inhibiting EMT said method comprising: contacting a test compound with a TARGET polypeptide, fragments or structurally functional derivatives thereof, determining a binding affinity of the test compound to said polypeptide or expression or an activity of said polypeptide, and identifying a compound inhibiting EMT as a compound which demonstrates a binding affinity to said polypeptide and/or is able to inhibit the expression or activity of said polypeptide.

The present invention also relates to:
a) pharmaceutical compositions comprising an antibody or a fragment thereof which specifically binds to a TARGET polypeptide, for use in the treatment of a disease associated with EMT.
b) pharmaceutical compositions comprising an agent selected from the group consisting of an antisense polynucleotide, a ribozyme, a small interfering RNA (siRNA) and a short-hairpin RNA (shRNA) for use in the treatment of a fibrotic condition, wherein said agent comprises a nucleic acid sequence complementary to, or engineered from, a naturally-occurring polynucleotide sequence of about 17 to about 30 contiguous nucleotides of a nucleic acid sequence selected encoding a TARGET polypeptide for use in the treatment of a disease associated with EMT.

Another aspect of this invention relates to an in vitro method of inhibiting EMT said method comprising contacting a population of epithelial cells with an inhibitor of the activity or expression of a TARGET polypeptide.

### BRIEF DESCRIPTION OF THE DRAWINGS

FIG. 1 shows a schematic overview of the EMT assay.

FIG. 2 shows the Inter-quartile Range (IQR) values for negative controls (N1, N2 and N3), positive controls (P1, P2, P3, P4 and P5) and samples (S) for both Fibronectin (FN) and methalloproteinase-10 (MMP10) read-outs for the complete primary screen. Dotted line indicates an IQR cut-off of −1.5.

FIG. 3 shows a rescreen plate layout, well G02 was mock transduced.

FIG. 4 shows Meso Scale Discovery platform (MSD) signal values in the rescreen for the controls and samples for both Fibronectin (FN) and methalloproteinase-10 (MMP10) read-outs. E: mock treated; S: samples.

FIG. 5 shows the validation plate layout. Well G02 contained no sample but was mock transduced for 9 source plates.

FIG. 6 shows the schematic assay overview of the on target screen with three read-outs: Fibronectin (FN), methalloproteinase-10 (MMP10) and CellTiter-Blue (CTB) fluorescence.

FIG. 7 shows the on target plate layout, well G02 was mock transduced in 12 of the 18 plates.

FIG. 8 shows the control performance in the “on target” screen for FN and MMP10, MSD signal is plotted. T−: no trigger, T+: trigger only and S: samples.

### DETAILED DESCRIPTION

The following terms are intended to have the meanings presented below and are useful in understanding the description and intended scope of the present invention.

The term ‘agent’ means any molecule, including polypeptides, polynucleotides, natural products and small molecules. In particular the term agent includes compounds such as test compounds or drug candidate compounds.

The term ‘activity inhibitory agent’ or ‘activity inhibiting agent’ means an agent, e.g. a polypeptide, small molecule, compound designed to interfere or capable of interfering selectively with the activity of a specific polypeptide or protein normally expressed within a cell.

The term ‘agonist’ refers to an agent that stimulates the receptor the agent binds to in the broadest sense.

As used herein, the term ‘antagonist’ is used to describe an agent that does not provoke a biological response itself upon binding to a receptor, but blocks or dampens agonist-mediated responses, or prevents or reduces agonist binding and, thereby, agonist-mediated responses.

The term ‘assay’ means any process used to measure a specific property of an agent, including a compound. A ‘screening assay’ means a process used to characterize or select compounds based upon their activity from a collection of compounds.

The term ‘binding affinity’ is a property that describes how strongly two or more compounds associate with each other in a non-covalent relationship. Binding affinities can be characterized qualitatively, (such as ‘strong’, ‘weak’, ‘high’, or low′) or quantitatively (such as measuring the KD).

The term ‘carrier’ means a non-toxic material used in the formulation of pharmaceutical compositions to provide a medium, bulk and/or useable form to a pharmaceutical composition. A carrier may comprise one or more of such materials such as an excipient, stabilizer, or an aqueous pH buffered solution. Examples of physiologically acceptable carriers include aqueous or solid buffer ingredients including phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

The term ‘complex’ means the entity created when two or more compounds bind to, contact, or associate with each other.

The term ‘compound’ is used herein in the context of a ‘test compound’ or a ‘drug candidate compound’ described in connection with the assays and methods of the present invention. As such, these compounds comprise organic or inorganic compounds, derived synthetically or from natural sources. The compounds include inorganic or organic compounds such as polynucleotides (e.g. siRNA or cDNA), lipids or hormone analogs. Other biopolymeric organic test compounds include peptides comprising from about 2 to about 40 amino acids and larger polypeptides comprising from about 40 to about 500 amino acids, including polypeptide ligands, enzymes, receptors, channels, antibodies or antibody conjugates.

The term ‘condition’ or ‘disease’ means the overt presentation of symptoms (i.e., illness) or the manifestation of abnormal clinical indicators (for example, biochemical or cellular indicators). Alternatively, the term ‘disease’ refers to a genetic or environmental risk of or propensity for developing such symptoms or abnormal clinical indicators.

The term ‘contact’ or ‘contacting’ means bringing at least two moieties together, whether in an in vitro system or an in vivo system.

The term ‘derivatives of a polypeptide’ relates to those peptides, oligopeptides, polypeptides, proteins and enzymes that comprise a stretch of contiguous amino acid residues of the polypeptide and that retain a biological activity of the protein, for example, polypeptides that have amino acid mutations compared to the amino acid sequence of a naturally-occurring form of the polypeptide. A derivative may further comprise additional naturally occurring, altered, glycosylated, acylated or non-naturally occurring amino acid residues compared to the amino acid sequence of a naturally occurring form of the polypeptide. It may also contain one or more non-amino acid substituents, or heterologous amino acid substituents, compared to the amino acid sequence of a naturally occurring form of the polypeptide, for example a reporter molecule or other ligand, covalently or non-covalently bound to the amino acid sequence.

The term ‘derivatives of a polynucleotide’ relates to DNA-molecules, RNA-molecules, and oligonucleotides that comprise a stretch of nucleic acid residues of the polynucleotide, for example, polynucleotides that may have nucleic acid mutations as compared to the nucleic acid sequence of a naturally occurring form of the polynucleotide. A derivative may further comprise nucleic acids with modified backbones such as PNA, polysiloxane, and 2′-O-(2-methoxy)ethyl-phosphorothioate, non-naturally occurring nucleic acid residues, or one or more nucleic acid substituents, such as methyl-, thio-, sulphate, benzoyl-, phenyl-, amino-, propyl-, chloro-, and methanocarbanucleosides, or a reporter molecule to facilitate its detection.

The term ‘endogenous’ shall mean a material that a mammal naturally produces. Endogenous in reference to the term ‘enzyme’, ‘protease’, ‘kinase’, or G-Protein Coupled Receptor (‘GPCR’) shall mean that which is naturally produced by a mammal (for example, and not by limitation, a human). In contrast, the term non-endogenous in this context shall mean that which is not naturally produced by a mammal (for example, and not by limitation, a human). Both terms can be utilized to describe both in vivo and in vitro systems. For example, and without limitation, in a screening approach, the endogenous or non-endogenous TARGET may be in reference to an in vitro screening system. As a further example and not limitation, where the genome of a mammal has been manipulated to include a non-endogenous TARGET, screening of a candidate compound by means of an in vivo system is feasible.

The term ‘expressible nucleic acid’ means a nucleic acid coding for or capable of encoding a proteinaceous molecule, peptide or polypeptide, and may include an RNA molecule, or a DNA molecule.

The term ‘expression’ comprises both endogenous expression and non-endogenous expression, including overexpression by transduction.

The term ‘expression inhibitory agent’ or ‘expression inhibiting agent’ means an agent, e.g. a polynucleotide designed to interfere or capable of interfering selectively with the transcription, translation and/or expression of a specific polypeptide or protein normally expressed within or by a cell. More particularly and by example, ‘expression inhibitory agent’ comprises a DNA or RNA molecule that contains a nucleotide sequence identical to or complementary to at least about 15-30, particularly at least 17, sequential nucleotides within the polyribonucleotide sequence coding for a specific polypeptide or protein. Exemplary such expression inhibitory molecules include ribozymes, microRNAs, double stranded siRNA molecules, self-complementary single-stranded siRNA molecules, genetic antisense constructs, and synthetic RNA antisense molecules with modified stabilized backbones.

The term “‘RNAi inhibitor” refers to any molecule that can down regulate, reduce or inhibit RNA interference function or activity in a cell or organism. An RNAi inhibitor can down regulate, reduce or inhibit RNAi (e.g., RNAi mediated cleavage of a target polynucleotide, translational inhibition, or transcriptional silencing) by interaction with or interfering with the function of any component of the RNAi pathway, including protein components such as RISC, or nucleic acid components such as miRNAs or siRNAs. A RNAi inhibitor can be an siNA molecule, an antisense molecule, an aptamer, or a small molecule that interacts with or interferes with the function of RISC, a miRNA, or an siRNA or any other component of the RNAi pathway in a cell or organism. By inhibiting RNAi (e.g., RNAi mediated cleavage of a target polynucleotide, translational inhibition, or transcriptional silencing), a RNAi inhibitor of the invention can be used to modulate (e.g., down regulate) the expression of a target gene.

The term “microRNA” or “miRNA” or “miR” as used herein refers to its meaning as is generally accepted in the art. More specifically, the term refers a small double-stranded RNA molecules that regulate the expression of target messenger RNAs either by mRNA cleavage, translational repression/inhibition or heterochromatic silencing (see for example Ambros, 2004, Nature, 431, 350-355; Barrel, 2004, Cell, 1 16, 281-297; Cullen, 2004, Virus Research., 102, 3-9; He et al, 2004, Nat. Rev. Genet., 5, 522-531; Ying el al, 2004, Gene, 342, 25-28; and Sethupathy et al, 2006, RNA, 12:192-197). As used herein, the term includes mature single stranded miRNAs, precursor miRNAs (pre-miR), and variants thereof, which may be naturally occurring. In some instances, the term “miRNA” also includes primary miRNA transcripts and duplex miRNAs.

The term ‘fragment of a polynucleotide’ relates to oligonucleotides that comprise a stretch of contiguous nucleic acid residues that exhibit substantially a similar, but not necessarily identical, activity as the complete sequence. In a particular aspect, ‘fragment’ may refer to a oligonucleotide comprising a nucleic acid sequence of at least 5 nucleic acid residues (preferably, at least 10 nucleic acid residues, at least 15 nucleic acid residues, at least 20 nucleic acid residues, at least 25 nucleic acid residues, at least 40 nucleic acid residues, at least 50 nucleic acid residues, at least 60 nucleic residues, at least 70 nucleic acid residues, at least 80 nucleic acid residues, at least 90 nucleic acid residues, at least 100 nucleic acid residues, at least 125 nucleic acid residues, at least 150 nucleic acid residues, at least 175 nucleic acid residues, at least 200 nucleic acid residues, or at least 250 nucleic acid residues) of the nucleic acid sequence of said complete sequence.

The term ‘fragment of a polypeptide’ relates to peptides, oligopeptides, polypeptides, proteins, monomers, subunits and enzymes that comprise a stretch of contiguous amino acid residues, and exhibit substantially a similar, but not necessarily identical, functional or expression activity as the complete sequence. In a particular aspect, ‘fragment’ may refer to a peptide or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of said complete sequence.

The term ‘hybridization’ means any process by which a strand of nucleic acid binds with a complementary strand through base pairing. The term ‘hybridization complex’ refers to a complex formed between two nucleic acid sequences by virtue of the formation of hydrogen bonds between complementary bases. A hybridization complex may be formed in solution (for example, COt or ROt analysis) or formed between one nucleic acid sequence present in solution and another nucleic acid sequence immobilized on a solid support (for example, paper, membranes, filters, chips, pins or glass slides, or any other appropriate substrate to which cells or their nucleic acids have been fixed). The term “stringent conditions” refers to conditions that permit hybridization between polynucleotides and the claimed polynucleotides. Stringent conditions can be defined by salt concentration, the concentration of organic solvent, for example, formamide, temperature, and other conditions well known in the art. In particular, reducing the concentration of salt, increasing the concentration of formamide, or raising the hybridization temperature can increase stringency. The term ‘standard hybridization conditions’ refers to salt and temperature conditions substantially equivalent to 5×SSC and 65° C. for both hybridization and wash. However, one skilled in the art will appreciate that such ‘standard hybridization conditions’ are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20NC below the predicted or determined Tm with washes of higher stringency, if desired.

The term ‘inhibit’ or ‘inhibiting’, in relationship to the term ‘response’ means that a response is decreased or prevented in the presence of a compound as opposed to in the absence of the compound.

The term ‘inhibition’ refers to the reduction, down regulation of a process or the elimination of a stimulus for a process, which results in the absence or minimization of the expression or activity of a protein or polypeptide.

The term ‘induction’ refers to the inducing, up-regulation, or stimulation of a process, which results in the expression, enhanced expression, activity, or increased activity of a protein or polypeptide.

The term ligand′ means an endogenous, naturally occurring molecule specific for an endogenous, naturally occurring receptor.

The term ‘pharmaceutically acceptable salts’ refers to the non-toxic, inorganic and organic acid addition salts, and base addition salts, of compounds which inhibit the expression or activity of TARGETS as disclosed herein. These salts can be prepared in situ during the final isolation and purification of compounds useful in the present invention.

The term ‘polypeptide’ relates to proteins (such as TARGETS), proteinaceous molecules, fragments of proteins, monomers or portions of polymeric proteins, peptides, oligopeptides and enzymes (such as kinases, proteases, GPCR's etc.).

The term ‘polynucleotide’ means a polynucleic acid, in single or double stranded form, and in the sense or antisense orientation, complementary polynucleic acids that hybridize to a particular polynucleic acid under stringent conditions, and polynucleotides that are homologous in at least about 60 percent of its base pairs, and more particularly 70 percent of its base pairs are in common, particularly 80 percent, most particularly 90 percent, and in a special embodiment 100 percent of its base pairs. The polynucleotides include polyribonucleic acids, polydeoxyribonucleic acids, and synthetic analogues thereof. It also includes nucleic acids with modified backbones such as peptide nucleic acid (PNA), polysiloxane, and 2′-O-(2-methoxy)ethylphosphorothioate. The polynucleotides are described by sequences that vary in length, that range from about 10 to about 5000 bases, particularly about 100 to about 4000 bases, more particularly about 250 to about 2500 bases. One polynucleotide embodiment comprises from about 10 to about 30 bases in length. A special embodiment of polynucleotide is the polyribonucleotide of from about 17 to about 22 nucleotides, more commonly described as small interfering RNAs (siRNAs—double stranded siRNA molecules or self-complementary single-stranded siRNA molecules (shRNA)). Another special embodiment are nucleic acids with modified backbones such as peptide nucleic acid (PNA), polysiloxane, and 2′-O-(2-methoxy)ethylphosphorothioate, or including non-naturally occurring nucleic acid residues, or one or more nucleic acid substituents, such as methyl-, thio-, sulphate, benzoyl-, phenyl-, amino-, propyl-, chloro-, and methanocarbanucleosides, or a reporter molecule to facilitate its detection. Polynucleotides herein are selected to be ‘substantially’ complementary to different strands of a particular target DNA sequence. This means that the polynucleotides must be sufficiently complementary to hybridize with their respective strands. Therefore, the polynucleotide sequence need not reflect the exact sequence of the target sequence. For example, a non-complementary nucleotide fragment may be attached to the 5′ end of the polynucleotide, with the remainder of the polynucleotide sequence being complementary to the strand. Alternatively, non-complementary bases or longer sequences can be interspersed into the polynucleotide, provided that the polynucleotide sequence has sufficient complementarity with the sequence of the strand to hybridize therewith under stringent conditions or to form the template for the synthesis of an extension product.

The term ‘preventing’ or ‘prevention’ refers to a reduction in risk of acquiring or developing a disease or disorder (i.e., causing at least one of the clinical symptoms of the disease not to develop) in a subject that may be exposed to a disease-causing agent, or predisposed to the disease in advance of disease onset.

The term ‘prophylaxis’ is related to and encompassed in the term ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

The term ‘subject’ includes humans and other mammals.

The term ‘TARGET’ or ‘TARGETS’ means the protein(s) identified in accordance with the assays described herein and determined to be involved in EMT. The term TARGET or TARGETS includes and contemplates alternative species forms, isoforms, and variants, such as splice variants, allelic variants, alternate in frame exons, and alternative or premature termination or start sites, including known or recognized isoforms or variants thereof such as indicated in Table 1. The NCBI accession numbers are provided to assist a skilled person to identify the transcripts and polypeptides. However, the term TARGET or TARGETS is not limited to those particular versions of the sequences and encompasses functional variants of nucleic acids and polypeptides corresponding to those sequences.

‘Therapeutically effective amount’ or ‘effective amount’ means that amount of a compound or agent that will elicit the biological or medical response in or of a subject that is being sought by or is accepted by a medical doctor or other clinician.

The term ‘treating’ or ‘treatment’ of any disease or disorder refers, in one embodiment, to ameliorating the disease or disorder (i.e., arresting the disease or reducing the manifestation, extent or severity of at least one of the clinical symptoms thereof). Accordingly, ‘treating’ refers to both therapeutic treatment and prophylactic or preventative measures. Those in need of treating include those already with the disorder as well as those in which the disorder is to be prevented. The related term ‘treatment,’ as used herein, refers to the act of treating a disorder, symptom, disease or condition. In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g., stabilization of a discernible symptom), physiologically, (e.g., stabilization of a physical parameter or of a physiologically measurable parameter), or both. In a further embodiment, ‘treating’ or ‘treatment’ relates to slowing the progression of the disease.

The term “vectors” also relates to plasmids as well as to viral vectors, such as recombinant viruses, or the nucleic acid encoding the recombinant virus.

The term “vertebrate cells” means cells derived from animals having vertebral structure, including fish, avian, reptilian, amphibian, marsupial, and mammalian species. Preferred cells are derived from mammalian species, and most preferred cells are human cells. Mammalian cells include feline, canine, bovine, equine, caprine, ovine, porcine, murine, such as mice and rats, and rabbits.

The term “EMT” or “epithelial mesenchymal transition” refers to a process that allows a polarized epithelial cell, which normally interacts with basement membrane via its basal surface, to undergo multiple biochemical changes that enable it to assume a mesenchymal cell phenotype, which includes enhanced migratory capacity, invasiveness, elevated resistance to apoptosis, and greatly increased production of ECM components.

The term “diseases related to EMT” refers to any condition or disease that has as one of the underlying causes the EMT process. Such diseases include, but not limited to, fibrotic diseases and cancer.

As used herein the term ‘fibrotic diseases’ refers to diseases characterized by excessive or persistent scarring, particularly due to excessive or abnormal production, deposition of extracellular matrix, and are that are associated with the abnormal accumulation of cells and/or fibronectin and/or collagen and/or increased fibroblast recruitment and include but are not limited to fibrosis of individual organs or tissues such as the heart, kidney, liver, joints, lung, pleural tissue, peritoneal tissue, skin, cornea, retina, musculoskeletal and digestive tract. In particular aspects, the term fibrotic diseases refers to idiopathic pulmonary fibrosis (IPF), cystic fibrosis, other diffuse parenchymal lung diseases of different etiologies including iatrogenic drug-induced fibrosis, occupational and/or environmental induced fibrosis, granulomatous diseases (sarcoidosis, hypersensitivity pneumonia), collagen vascular disease, alveolar proteinosis, langerhans cell granulomatosis, lymphangioleiomyomatosis, inherited diseases (Hermansky-Pudlak Syndrome, tuberous sclerosis, neurofibromatosis, metabolic storage disorders, familial interstitial lung disease), radiation induced fibrosis, chronic obstructive pulmonary disease (COPD), scleroderma, bleomycin induced pulmonary fibrosis, chronic asthma, silicosis, asbestos induced pulmonary fibrosis, acute respiratory distress syndrome (ARDS), kidney fibrosis, tubulointerstitium fibrosis, glomerular nephritis, focal segmental glomerular sclerosis, IgA nephropathy, hypertension, Alport syndrome, gut fibrosis, liver fibrosis, cirrhosis, alcohol induced liver fibrosis, toxic/drug induced liver fibrosis, hemochromatosis, nonalcoholic steatohepatitis (NASH), biliary duct injury, primary biliary cirrhosis, infection induced liver fibrosis, viral induced liver fibrosis, autoimmune hepatitis, corneal scarring, hypertrophic scarring, Dupuytren disease, keloids, cutaneous fibrosis, cutaneous scleroderma, systemic sclerosis, spinal cord injury/fibrosis, myelofibrosis, vascular restenosis, atherosclerosis, arteriosclerosis, Wegener's granulomatosis and Peyronie's disease. More particularly, the term “fibrotic diseases” refers to idiopathic pulmonary fibrosis (IPF).

As used herein, the term ‘cancer’ refers to a malignant or benign growth of cells in skin or in body organs, for example but without limitation, breast, prostate, lung, kidney, pancreas, stomach or bowel. A cancer tends to infiltrate into adjacent tissue and spread (metastasise) to distant organs, for example to bone, liver, lung or the brain. As used herein the term cancer includes both metastatic tumour cell types (such as but not limited to, melanoma, lymphoma, leukaemia, fibrosarcoma, rhabdomyosarcoma, and mastocytoma) and types of tissue carcinoma (such as but not limited to, colorectal cancer, prostate cancer, small cell lung cancer and non-small cell lung cancer, breast cancer, pancreatic cancer, bladder cancer, renal cancer, gastric cancer, glioblastoma, primary liver cancer, ovarian cancer, prostate cancer and uterine leiomyosarcoma). In particular, the term “cancer” refers to acute lymphoblastic leukemia, acute myeloidleukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, embryonal tumors, endometrial cancer, ependymoblastoma, ependymoma, esophageal cancer, ewing sarcoma family of tumors, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, Acute lymphoblastic leukemia, acute myeloid leukemia, chronic lymphocytic leukemia, chronic myelogenous leukemia, hairy cell leukemia, liver cancer, non-small cell lung cancer, small cell lung cancer, Burkitt lymphoma, cutaneous T-cell lymphoma, Hodgkin lymphoma, non-Hodgkin lymphoma, lymphoma, Waldenstrom macroglobulinemia, medulloblastoma, medulloepithelioma, melanoma, mesothelioma, mouth cancer, chronic myelogenous leukemia, myeloid leukemia, multiple myeloma, asopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, retinoblastoma, rhabdomyosarcoma, salivary gland cancer, sarcoma, Ewing sarcoma family of tumors, sarcoma, kaposi, Sezary syndrome, skin cancer, small cell Lung cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, and Wilms tumor. More specifically the term “cancer” includes melanoma, lymphoma, leukaemia, fibrosarcoma, rhabdomyosarcoma, mastocytoma, colorectal cancer, prostate cancer, small cell lung cancer and non-small cell lung cancer, breast cancer, pancreatic cancer, bladder cancer, renal cancer, gastric cancer, glioblastoma, primary liver cancer, ovarian cancer, prostate cancer and uterine leiomyosarcoma. In more specific aspect the term “cancer’ is related to a cancer associated and/or correlated with EMT, more specifically cancer metastasis.

### Targets

Applicant's invention is relevant to the treatment, prevention and alleviation of conditions and disorders associated with EMT, more particular with fibrotic diseases and cancer.

The present invention is based on extensive work by the present inventors to develop an in vitro (cell-free or cell based) assay system suitable to provide a scientifically valid substitute for the naturally occurring in vivo process of epithelial mesenchymal transition (EMT). The process of EMT is known to be involved in fibrosis and cancer development; however it is a complex process. The present invention provides an artificial model for the natural system using distinct and quantifiable in vitro parameters, which is suitable for the identification of compounds inhibiting EMT, and, thus, identify compounds that may be useful in the treatment and/or prevention of fibrosis and carcinomas.

The present invention provides methods for assaying for drug candidate compounds useful in treatment of diseases associated with EMT, particular useful in reducing and/or inhibiting EMT comprising contacting the compound with a cell expressing a TARGET, and determining the relative amount or degree of inhibition of EMT in the presence and/or absence of the compound. The present invention provides methods for assaying for drug candidate compounds useful in treatment of diseases associated with EMT, particularly useful in reducing and/or inhibiting EMT, comprising contacting the compound with a cell expressing a TARGET, and determining the relative amount or degree of inhibition of the expression or activity of the TARGET, whereby inhibition of expression or activity of the TARGET is associated with or results in inhibition of or reduced EMT in the presence and/or absence of the compound. Such methods may be used to identify target proteins that act to inhibit said transition; alternatively, they may be used to identify compounds that down-regulate or inhibit the expression or activity of TARGET proteins. The invention provides methods for assaying for drug candidate compounds useful in the treatment of fibrosis, comprising contacting the compound with a TARGET, under conditions wherein the expression or activity of the TARGET may be measured, and determining whether the TARGET expression or activity is altered in the presence of the compound, contacting a population of epithelial cells with said test compound and measuring a property related to EMT. Exemplary such methods can be designed and determined by the skilled artisan. Particular such exemplary methods are provided herein.

The present invention is based on the inventors' discovery that the TARGET polypeptides and their encoding nucleic acids, identified as a result of screens described below in the Examples, are factors involved in the fibrosis and in particular in EMT. A reduced activity or expression of the TARGET polypeptides and/or their encoding polynucleotides is causative, correlative or associated with reduced or inhibited EMT. Alternatively, a reduced activity or expression of the TARGET polypeptides and/or their encoding polynucleotides is causative, correlative or associated with decrease of the markers of EMT.

In a particular embodiment of the invention, the TARGET polypeptide comprises an amino acid sequence selected from the group consisting of SEQ ID NO: 18-34 as listed in Table 1.

TABLE 1

| Target Gene | GenBank | SEQ ID NO: | GenBank | SEQ ID NO: | | |
|---|---|---|---|---|---|---|
| Symbol | Nucleic Acid Acc #: | DNA | Protein Acc # | Protein | NAME | Class |
| CLK2 | NM_003993.2 | 1 | NP_003984.2 | 18 | CDC-like kinase 2 | Kinase |
| CSNK2A2 | NM_001896.2 | 2 | NP_001887.1 | 19 | casein kinase 2, alpha | Kinase |
| | | | | | prime polypeptide | |
| PARP1 | NM_001618.3 | 3 | NP_001609.2 | 20 | poly (ADP-ribose) | Transferase |
| | | | | | polymerase 1 | |
| IGFBP7 | NM_001553.2 | 4 | NP_001544.1 | 21 | insulin-like growth factor | Secreted/ |
| | NM_001253835.1 | 5 | NP_001240764.1 | 22 | binding protein 7 | Extracellular |
| APOL1 | NM_003661.3 | 6 | NP_003652.2 | 23 | apolipoprotein L, 1 | Secreted/ |
| | NM_145343.2 | 7 | NP_663318.1 | 24 | | Extracellular |
| | NM_001136540.1 | 8 | NP_001130012.1 | 25 | | |
| | NM_001136541.1 | 9 | NP_001130013.1 | 26 | | |
| STK4 | NM_006282.2 | 10 | NP_006273.1 | 27 | serine/threonine kinase 4 | Kinase |
| OTUD6B | NM_016023.3 | 11 | NP_057107.3 | 28 | OTU domain containing 6B | Unknown |
| ADRBK2 | NM_005160.3 | 12 | NP_005151.2 | 29 | adrenergic, beta, receptor | Kinase |
| | | | | | kinase 2 | |
| EFEMP2 | NM_016938.4 | 13 | NP_058634.4 | 30 | EGF containing fibulin- | Receptor |
| | | | | | like extracellular matrix | |
| | | | | | protein 2 | |
| F2R | NM_001992.3 | 14 | NP_001983.2 | 31 | coagulation factor II | GPCR |
| | | | | | (thrombin) receptor | |
| SLC15A3 | NM_016582.2 | 15 | NP_057666.1 | 32 | solute carrier family 15, | Transporter |
| | | | | | member 3 | |
| WNT5A | NM_003392.4 | 16 | NP_003383.2 | 33 | wingless-type MMTV | Secreted/ |
| | NM_001256105.1 | 17 | NP_001243034.1 | 34 | integration site family, | Extracellular |
| | | | | | member 5A | |

A particular embodiment of the invention comprises the kinase TARGETs identified as SEQ ID NO: 18, 19, 27 and 29. A particular embodiment of the invention comprises the transferase TARGET identified as SEQ ID NO: 20. A particular embodiment of the invention comprises the secreted/extracellular TARGETs identified as SEQ ID NO: 21-22, 23-26 and 33-34. A particular embodiment of the invention comprises the receptor TARGET identified as SEQ ID NO: 30. A particular embodiment of the invention comprises the GPCR TARGET identified as SEQ ID NO: 31. A particular embodiment of the invention comprises the transporter TARGET identified as SEQ ID NO: 32.

### Methods of the Invention

In one aspect, the present invention relates to a method for identifying a compound useful for the treatment of a disease associated with epithelial mesenchymal transition (EMT), said method comprising:
a) contacting a test compound with a polypeptide comprising an amino acid sequence selected from the group consisting of SEQ ID NOs: 18-34, fragments and functional derivatives thereof;
b) measuring a binding affinity of the test compound to said polypeptide;
c) contacting the test compound with a population of epithelial cells;
d) measuring a property related to EMT; and
e) identifying a compound inhibiting EMT and demonstrating binding affinity to said polypeptide.

In further aspect, the present invention relates to a method for identifying a compound inhibiting epithelial mesenchymal transition (EMT), said method comprising:
a) contacting a test compound with a polypeptide comprising an amino acid sequence selected from the group consisting of SEQ ID NOs: 18-34, fragments and functional derivatives thereof;
b) measuring a binding affinity of the test compound to said polypeptide;
c) contacting the test compound with a population of epithelial cells;
d) measuring a property related to EMT; and
e) identifying a compound inhibiting EMT and demonstrating binding affinity to said polypeptide.

In one aspect, the present invention relates to a method for identifying a compound that inhibits epithelial mesenchymal transition (EMT), said method comprising:
a) contacting a test compound with a polypeptide comprising an amino acid sequence selected from the group consisting of SEQ ID NOs: 18-34, fragments and functional derivatives thereof or with a nucleic acid encoding an amino acid selected from the group consisting of SEQ ID NOs: 18-34 or a functional derivative thereof;
b) identifying and/or measuring a binding affinity of the test compound to said polypeptide or nucleic acid;
c) contacting the test compound with a population of epithelial cells;
d) measuring a property related to or indicating inhibition or reduction of EMT; and
e) identifying a compound inhibiting or reducing EMT and demonstrating binding affinity to said polypeptide or nucleic acid.

In a further aspect of the above method, the nucleic acid encoding an amino acid selected from the group consisting of SEQ ID NOs: 18-34 or a functional derivative thereof may be selected from the group consisting of SEQ ID NOs: 1-17.

The order of taking these measurements is not believed to be critical to the practice of the present invention, which may be practiced in any order. In a particular aspect the method steps (c) and (d) may be performed before performing steps (a) and (b). For example, one may first perform a screening assay of a set of compounds for which no information is known respecting the compounds' binding affinity for the polypeptide. Alternatively, one may screen a set of compounds identified as having binding affinity for a polypeptide domain, or a class of compounds identified as being an inhibitor of the polypeptide.

In another aspect, steps (a)-(d) method may also be performed simultaneously in a cell-based assay by contacting a test compound with a population of macrophages, measuring a binding affinity of the test compound to a TARGET polypeptide and a property related to epithelial mesenchymal transition, and identifying a compound capable of inhibiting epithelial mesenchymal transition and which demonstrates binding affinity to said polypeptide.

The binding affinity of a compound with the polypeptide TARGET can be measured by methods known in the art, such as using surface plasmon resonance biosensors (Biacore), by saturation binding analysis with a labeled compound (for example, Scatchard and Lindmo analysis), by differential UV spectrophotometer, fluorescence polarization assay, Fluorometric Imaging Plate Reader (FLIPR®) system, Fluorescence resonance energy transfer, and Bioluminescence resonance energy transfer. The binding affinity of compounds can also be expressed in dissociation constant (Kd) or as IC₅₀ or EC₅₀. The IC₅₀ represents the concentration of a compound that is required for 50% inhibition of binding of another ligand to the polypeptide. The EC₅₀ represents the concentration required for obtaining 50% of the maximum effect in any assay that measures TARGET function. The dissociation constant, Kd, is a measure of how well a ligand binds to the polypeptide, it is equivalent to the ligand concentration required to saturate exactly half of the binding-sites on the polypeptide. Compounds with a high affinity binding have low Kd, IC₅₀ and EC₅₀ values, for example, in the range of 100 nM to 1 pM; a moderate- to low-affinity binding relates to high Kd, IC₅₀ and EC₅₀ values, for example in the micromolar range.

In one aspect, the assay method includes contacting a TARGET polypeptide with a compound that exhibits a binding affinity in the micromolar range. In an aspect, the binding affinity exhibited is at least 10 micromolar. In an aspect, the binding affinity is at least 1 micromolar. In an aspect, the binding affinity is at least 500 nanomolar.

In a particular aspect a test compound is selected based on its ability to bind to a TARGET class or from known libraries of compounds having ability to bind to a TARGET class.

In further aspect, the present invention relates to a method for identifying a compound useful for the treatment of a disease associated with epithelial mesenchymal transition (EMT), said method comprising:
a) contacting a test compound with a polypeptide comprising an amino acid sequence selected from the group consisting of SEQ ID NOs: 18-34, functional fragments and functional derivatives thereof;
b) measuring an activity of said polypeptide;
c) contacting the test compound with a population of epithelial cells;
d) measuring a property related to epithelial mesenchymal transition; and
e) identifying a compound inhibiting epithelial mesenchymal transition and inhibiting the activity of said polypeptide.

In an additional aspect, the present invention relates to a method for identifying a compound inhibiting epithelial mesenchymal transition (EMT), said method comprising:
a) contacting a test compound with a polypeptide comprising an amino acid sequence selected from the group consisting of SEQ ID NOs: 18-34, functional fragments and functional derivatives thereof;
b) measuring an activity of said polypeptide;
c) contacting the test compound with a population of epithelial cells;
d) measuring a property related to EMT; and
e) identifying a compound inhibiting EMT and inhibiting the activity of said polypeptide.

In a further aspect, the present invention relates to a method for identifying a compound inhibiting epithelial mesenchymal transition (EMT), said method comprising:
a) contacting a test compound with a polypeptide comprising an amino acid sequence selected from the group consisting of SEQ ID NOs: 18-34, functional fragments and functional derivatives thereof or with a nucleic acid encoding an amino acid selected from the group consisting of SEQ ID NOs: 18-34 or a functional derivative thereof;
b) measuring the expression or an activity of said polypeptide;
c) identifying a compound capable of inhibiting the expression or activity of said polypeptide whereby inhibition of expression or activity of said polypeptide results in or is associated with inhibition or reduction of EMT.

In an additional aspect of the above method, the nucleic acid encoding an amino acid selected from the group consisting of SEQ ID NOs: 18-34 or a functional derivative thereof may be selected from the group consisting of SEQ ID NOs: 1-17.

The order of taking these measurements is not believed to be critical to the practice of the present invention, which may be practiced in any order. In a particular aspect of the method steps (c) and (d) may be performed before performing steps (a) and (b). For example, one may first perform a screening assay of a set of compounds for which no information is known respecting the compounds' binding affinity for the polypeptide. Alternatively, one may screen a set of compounds identified as having binding affinity for a polypeptide domain, or a class of compounds identified as being an inhibitor of the polypeptide.

Table 1 lists the TARGETS identified using applicants' knock-down library in the EMT assay exemplified herein, including the class of polypeptides identified. TARGETS have been identified in polypeptide classes including kinases, proteases, enzymes, ion channels, GPCRs, and extracellular proteins, for instance. A skilled artisan would be aware of different methods of measuring activity of those classes both in cell-free preparations as well in cell-based assays. A variety of methods exists and might be adapted to a particular target. Those adaptations are a matter of routine experimentation and rely on the existent techniques and methods. Some exemplary methods are described herein.

Ion channels are membrane protein complexes and their function is to facilitate the diffusion of ions across biological membranes. Membranes, or phospholipid bilayers, build a hydrophobic, low dielectric barrier to hydrophilic and charged molecules. Ion channels provide a high conducting, hydrophilic pathway across the hydrophobic interior of the membrane. The activity of an ion channel can be measured using classical patch clamping. High-throughput fluorescence-based or tracer-based assays are also widely available to measure ion channel activity. These fluorescent-based assays screen compounds on the basis of their ability to either open or close an ion channel thereby changing the concentration of specific fluorescent dyes across a membrane. In the case of the tracer-based assay, the changes in concentration of the tracer within and outside the cell are measured by radioactivity measurement or gas absorption spectrometry.

Specific methods to determine the inhibition by the compound by measuring the cleavage of the substrate by the polypeptide, which is a protease, are well known in the art. Classically, substrates are used in which a fluorescent group is linked to a quencher through a peptide sequence that is a substrate that can be cleaved by the target protease. Cleavage of the linker separates the fluorescent group and quencher, giving rise to an increase in fluorescence.

G-protein coupled receptors (GPCR) are capable of activating an effector protein, resulting in changes in second messenger levels in the cell. The TARGET(s) represented by SEQ ID NO: 31 are GPCR(s). The activity of a GPCR can be measured by measuring the activity level of such second messengers. Two important and useful second messengers in the cell are cyclic AMP (cAMP) and Ca²⁺. The activity levels can be measured by methods known to persons skilled in the art, either directly by ELISA or radioactive technologies or by using substrates that generate a fluorescent or luminescent signal when contacted with Ca²⁺ or indirectly by reporter gene analysis. The activity level of the one or more secondary messengers may typically be determined with a reporter gene controlled by a promoter, wherein the promoter is responsive to the second messenger. Promoters known and used in the art for such purposes are the cyclic-AMP responsive promoter that is responsive for the cyclic-AMP levels in the cell, and the NF-AT responsive promoter that is sensitive to cytoplasmic Ca²⁺-levels in the cell. The reporter gene typically has a gene product that is easily detectable. The reporter gene can either be stably infected or transiently transfected in the host cell. Useful reporter genes are alkaline phosphatase, enhanced green fluorescent protein, destabilized green fluorescent protein, luciferase and β-galactosidase.

In an another aspect the present relation relates to a method for identifying a compound useful for the treatment of a disease associated with epithelial mesenchymal transition (EMT), said method comprising
a) contacting a test compound with population of epithelial cells and expressing a polypeptide comprising an amino acid sequence selected from the group consisting of SEQ ID NOs: 18-34;
b) measuring expression, activity and/or amount of said polypeptide in said cells;
c) measuring a property related to EMT; and
d) identifying a compound producing reduction of expression and/or amount of said polypeptide and inhibiting or reducing EMT.

In a further aspect the present relation relates to a method for identifying a compound inhibiting epithelial mesenchymal transition (EMT), said method comprising
a) contacting a test compound with population of epithelial cells and expressing a polypeptide comprising an amino acid sequence selected from the group consisting of SEQ ID NOs: 18-34;
b) measuring expression, activity and/or amount of said polypeptide in said cells;
c) measuring a property related to EMT; and
d) identifying a compound producing reduction of expression and/or amount of said polypeptide and inhibiting EMT.

In particular aspect the method steps of the invention related to measuring of binding to a TARGET or activity are performed with a population of mammalian cells, in particular human cells, which have been engineered so as to express said TARGET polypeptide. In an alternative aspect the methods of the invention are performed using a population of epithelial cells, which have been engineered so as to express said TARGET polypeptide. This can be achieved by expression of the TARGET polypeptide in the cells using appropriate techniques known to a skilled person. In a specific embodiment, this can be achieved by over-expression of the TARGET polypeptide in the cells using appropriate techniques known to a skilled person. Alternatively, the method of the invention maybe performed with a population of macrophages which are known to naturally express said TARGET polypeptide.

In particular aspect the measurements of expression and/or amount of a TARGET polypeptide and a measurement of a property related to epithelial mesenchymal transition can be done in separate steps using different populations of macrophage cells. The measurements in steps (b) and (c) can also be performed in reverse order. The order of taking these measurements is not believed to be critical to the practice of the present invention, which may be practiced in any order.

In a specific embodiment the methods of the invention are used for identifying a compound useful for the treatment of fibrotic conditions characterized by aberrant epithelial mesenchymal transition.

In another embodiment the methods of the invention are used for identifying a compound useful for the treatment of cancers characterized by aberrant epithelial mesenchymal transition

One particular means of measuring the activity or expression of the polypeptide is to determine the amount of said polypeptide using a polypeptide binding agent, such as an antibody, or to determine the activity of said polypeptide in a biological or biochemical measure, for instance the amount of phosphorylation of a target of a kinase polypeptide.

TARGET gene expression (mRNA levels) can be measured using techniques well-known to a skilled artisan. Particular examples of such techniques include northern analysis or real-time PCR. Those methods are indicative of the presence of nucleic acids encoding TARGETs in a sample, and thereby correlate with expression of the transcript from the polynucleotide.

The population of cells may be exposed to the compound or the mixture of compounds through different means, for instance by direct incubation in the medium, or by nucleic acid transfer into the cells. Such transfer may be achieved by a wide variety of means, for instance by direct transfection of naked isolated DNA, or RNA, or by means of delivery systems, such as recombinant vectors. Other delivery means such as liposomes, or other lipid-based vectors may also be used. Particularly, the nucleic acid compound is delivered by means of a (recombinant) vector such as a recombinant virus.

In vivo animal models of fibrosis may be utilized by the skilled artisan to further or additionally screen, assess, and/or verify the agents or compounds identified in the present invention, including further assessing TARGET modulation in vivo. Such animal models include, but are not limited to, Bleomycin, irradiation, silica, (inducible) transgenic mouse, FITC and adoptive transfer models for lung fibrosis (Moore et al., 2008), COL4A3-deficiency, nephrotoxic serum nephritis and unilateral ureteral obstruction models for renal fibrosis (Zeisberg et al, 2005) and CCL4 intoxication model for liver fibrosis (Starkel et al., 2011)

A population of epithelial cells in the methods of the invention does not have to be pure or requires a particular degree of purity. A population of mammalian cells wherein some of said cells are epithelial cells is sufficient to practice the methods of present invention. The number or amount of macrophage cells should be sufficient to determine whether there are significant or relevant changes in EMT, or should be sufficient to evaluate differences, such as a significant decrease or increase, in an EMT marker or factor. It should be understood that a population of epithelial cells can be also obtained directly from an organ or alternatively grown using an appropriate medium. The techniques of generating a population of epithelial cells are known to a person skilled in the art.

In specific embodiment the methods may additionally comprise the step of comparing the compound to be tested to a control. Suitable controls should always be in place to insure against false positive readings. In a particular embodiment of the present invention the screening method comprises the additional step of comparing the compound to a suitable control. In one embodiment, the control may be a cell or a sample that has not been in contact with the test compound. In an alternative embodiment, the control may be a cell that does not express the TARGET; for example in one aspect of such an embodiment the test cell may naturally express the TARGET and the control cell may have been contacted with an agent, e.g. an siRNA, which inhibits or prevents expression of the TARGET. Alternatively, in another aspect of such an embodiment, the cell in its native state does not express the TARGET and the test cell has been engineered so as to express the TARGET, so that in this embodiment, the control could be the untransformed native cell. The control may also alternatively utilize a known inhibitor of epithelial mesenchymal transition or a compound known not to have any significant effect on epithelial mesenchymal transition. Whilst exemplary controls are described herein, this should not be taken as limiting; it is within the scope of a person of skill in the art to select appropriate controls for the experimental conditions being used.

Examples of negative controls include, but not limited to, cells that have been not treated with any compound, cells treated with a compound known not to be an inhibitor of EMT, compounds known not to interfere with the pathways involved in EMT. Examples of positive controls include, but not limited to, cells contacted with compounds known to inhibit activity or expression of SMAD3, SMAD4, TGFβR, Fibronectin, cells contacted with a compound known to inhibit TGFβ receptor signaling. In a particular embodiment the binding and activity testing in the invention methods is performed in an in vitro cell-free preparation.

In an alternative embodiment the binding and activity testing in the invention methods is performed in a cell.

In a particular aspect the invention methods activity and binding testing is performed in a mammalian cell, particularly a human cell. More specifically these steps are performed in epithelial cells. In a specific embodiment said cells are bronchial epithelial cells.

It should be understood that the cells expressing the polypeptides, may be cells naturally expressing the polypeptides, or the cells may be may be transfected to express the polypeptides. Also, the cells may be transduced to overexpress the polypeptide, or may be transfected to express a non-endogenous form of the polypeptide, which can be differentially assayed or assessed.

The polynucleotide expressing the TARGET polypeptide in cells might be included within a vector. The polynucleic acid is operably linked to signals enabling expression of the nucleic acid sequence and is introduced into a cell utilizing, particularly, recombinant vector constructs, which will express the nucleic acid once the vector is introduced into the cell. A variety of viral-based systems are available, including adenoviral, retroviral, adeno-associated viral, lentiviral, herpes simplex viral or a sendai viral vector systems. All may be used to introduce and express a TARGET polypeptide in the target cells.

In a particular embodiment the assay methods of the invention involve measurement of the inhibition of release or expression of a marker of epithelial mesenchymal transition (EMT marker).

Many of the EMT markers are known to a skilled person. The selection of such markers depends on the availability of reagents, scale of the practiced assay methods and other factors related to a specific assay design. In a specific embodiment an EMT marker is selected from the group consisting of Matrix Metalloproteases (MMPs), cellular fibronectin (FN), E-cadherin, soluble fibronectin, and vimentin. In a specific embodiment the EMT marker is selected from the group consisting of MMP10, fibronectin, E-cadherin and soluble fibronectin.

The means of measuring such markers, depending on the assay setup and throughput, are known to a skilled artisan. Although human ELISA's are commercially available their sensitivity is not always sufficient to detect low levels of the markers. Therefore, the assay might be optimized on the Meso Scale Discovery platform (MSD) (Meso Scale Discovery, Maryland, US) as a sandwich immunoassay where signaling molecules are specifically captured and detected by antibodies. MSD technology uses micro-plates with carbon electrodes integrated at the bottom of the plates; Biological reagents, immobilized to the carbon simply by passive adsorption, retain high biological activity. MSD assays use electro-chemiluminescent labels for ultra-sensitive detection. The detection process is initiated at electrodes located at the bottom of the micro-plates. Labels near the electrode only are excited and detected reducing background signal. The antibodies for such assay might be purchased from different producers and the skilled artisan is in the position to choose correct antibodies to perform the assay.

Alternatively the expression levels of the EMT markers can be measured using known methods including quantitative real time polymerase chain reaction (Q-PCR/qPCR/qrt-PCR). qPCR is a laboratory technique based on the PCR, which is used to amplify and simultaneously quantify a targeted DNA molecule. For one or more specific sequences in a DNA sample, Real Time-PCR enables both detection and quantification. The quantity can be either an absolute number of copies or a relative amount when normalized to DNA input or additional normalizing genes

In a specific embodiment the methods of the invention utilize cells that have been triggered by a factor which induces EMT (EMT inducing factor). Many of such factors have been described in literature and they are well-known to a skilled person. In a particular embodiment the methods of the invention utilize cells that have been triggered by one or more EMT inducing factors selected from the group consisting of TGFβ, IL-1β, TNFα, and a bacterial challenge. Bacterial challenge is the exposure of cells to UV killed bacteria in order to mimic bacterial insults occurring in vivo and may affect the fibrotic process.

In more particular embodiment the assay methods are performed using cells that have been triggered by a combination of TGFβ, TNFα and non-typeable Haemophilus influenzae.

### Candidate Compounds

### Expression-Inhibiting Agents

In a particular embodiment the methods of the invention a test compound is selected from the group consisting of an antisense polynucleotide, a ribozyme, short-hairpin RNA (shRNA), microRNA (miRNA) and a small interfering RNA (siRNA). 1001161A special embodiment of these methods comprises the expression-inhibitory agent selected from the group consisting of antisense RNA, antisense oligodeoxynucleotide (ODN), a ribozyme that cleaves the polyribonucleotide coding for SEQ ID NO: 18-34, a small interfering RNA (siRNA) or microRNA (miRNA) that is sufficiently homologous to a portion of the polyribonucleotide corresponding to SEQ ID NO: 1-17, such that the expression-inhibitory agent interferes with the translation of the TARGET polyribonucleotide to the TARGET polypeptide.

The down regulation of gene expression using antisense nucleic acids can be achieved at the translational or transcriptional level. Antisense nucleic acids of the invention are particularly nucleic acid fragments capable of specifically hybridizing with all or part of a nucleic acid encoding a TARGET polypeptide or the corresponding messenger RNA. In addition, antisense nucleic acids may be designed which decrease expression of the nucleic acid sequence capable of encoding a TARGET polypeptide by inhibiting splicing of its primary transcript. Any length of antisense sequence is suitable for practice of the invention so long as it is capable of down-regulating or blocking expression of a nucleic acid coding for a TARGET. Particularly, the antisense sequence is at least about 15-30, and particularly at least 17 nucleotides in length. The preparation and use of antisense nucleic acids, DNA encoding antisense RNAs and the use of oligo and genetic antisense is known in the art.

In a more specific embodiment a test compound comprises a nucleic acid sequence complementary to, or engineered from, a naturally-occurring polynucleotide sequence of about 17 to about 30 contiguous nucleotides of a TARGET polynucleotide.

The skilled artisan can readily utilize any of several strategies to facilitate and simplify the selection process for antisense nucleic acids and oligonucleotides effective in inhibition of TARGET and differentiation of macrophages into alternatively-activated macrophages. Predictions of the binding energy or calculation of thermodynamic indices between an oligonucleotide and a complementary sequence in an mRNA molecule may be utilized (Chiang et al. (1991) J. Biol. Chem. 266:18162-18171; Stull et al. (1992) Nucl. Acids Res. 20:3501-3508). Antisense oligonucleotides may be selected on the basis of secondary structure (Wickstrom et al (1991) in Prospects for Antisense Nucleic Acid Therapy of Cancer and AIDS, Wickstrom, ed., Wiley-Liss, Inc., New York, pp. 7-24; Lima et al. (1992) Biochem. 31:12055-12061). Schmidt and Thompson (U.S. Pat. No. 6,416,951) describe a method for identifying a functional antisense agent comprising hybridizing an RNA with an oligonucleotide and measuring in real time the kinetics of hybridization by hybridizing in the presence of an intercalation dye or incorporating a label and measuring the spectroscopic properties of the dye or the label's signal in the presence of unlabelled oligonucleotide. In addition, any of a variety of computer programs may be utilized which predict suitable antisense oligonucleotide sequences or antisense targets utilizing various criteria recognized by the skilled artisan, including for example the absence of self-complementarity, the absence of hairpin loops, the absence of stable homodimer and duplex formation (stability being assessed by predicted energy in kcal/mol). Examples of such computer programs are readily available and known to the skilled artisan and include the OLIGO 4 or OLIGO 6 program (Molecular Biology Insights, Inc., Cascade, Colo.) and the Oligo Tech program (Oligo Therapeutics Inc., Wilsonville, Oreg.). In addition, antisense oligonucleotides suitable in the present invention may be identified by screening an oligonucleotide library, or a library of nucleic acid molecules, under hybridization conditions and selecting for those which hybridize to the target RNA or nucleic acid (see for example U.S. Pat. No. 6,500,615). Mishra and Toulme have also developed a selection procedure based on selective amplification of oligonucleotides that bind target (Mishra et al (1994) Life Sciences 317:977-982). Oligonucleotides may also be selected by their ability to mediate cleavage of target RNA by RNAse H, by selection and characterization of the cleavage fragments (Ho et al (1996) Nucl Acids Res 24:1901-1907; Ho et al (1998) Nature Biotechnology 16:59-630). Generation and targeting of oligonucleotides to GGGA motifs of RNA molecules has also been described (U.S. Pat. No. 6,277,981).

The antisense nucleic acids are particularly oligonucleotides and may consist entirely of deoxyribo-nucleotides, modified deoxyribonucleotides, or some combination of both. The antisense nucleic acids can be synthetic oligonucleotides. The oligonucleotides may be chemically modified, if desired, to improve stability and/or selectivity. Specific examples of some particular oligonucleotides envisioned for this invention include those containing modified backbones, for example, phosphorothioates, phosphotriesters, methyl phosphonates, short chain alkyl or cycloalkyl intersugar linkages or short chain heteroatomic or heterocyclic intersugar linkages. Since oligonucleotides are susceptible to degradation by intracellular nucleases, the modifications can include, for example, the use of a sulfur group to replace the free oxygen of the phosphodiester bond. This modification is called a phosphorothioate linkage. Phosphorothioate antisense oligonucleotides are water soluble, polyanionic, and resistant to endogenous nucleases. In addition, when a phosphorothioate antisense oligonucleotide hybridizes to its TARGET site, the RNA-DNA duplex activates the endogenous enzyme ribonuclease (RNase) H, which cleaves the mRNA component of the hybrid molecule. Oligonucleotides may also contain one or more substituted sugar moieties. Particular oligonucleotides comprise one of the following at the 2′ position: OH, SH, SCH3, F, OCN, heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. Similar modifications may also be made at other positions on the oligonucleotide, particularly the 3′ position of the sugar on the 3′ terminal nucleotide and the 5′ position of 5′ terminal nucleotide.

Ih addition, antisense oligonucleotides with phosphoramidite and polyamide (peptide) linkages can be synthesized. These molecules should be very resistant to nuclease degradation. Furthermore, chemical groups can be added to the 2′ carbon of the sugar moiety and the 5 carbon (C-5) of pyrimidines to enhance stability and facilitate the binding of the antisense oligonucleotide to its TARGET site. Modifications may include 2′-deoxy, O-pentoxy, O-propoxy, O-methoxy, fluoro, methoxyethoxy phosphorothioates, modified bases, as well as other modifications known to those of skill in the art.

Another type of expression-inhibitory agent that reduces the levels of TARGETS is the ribozyme. Ribozymes are catalytic RNA molecules (RNA enzymes) that have separate catalytic and substrate binding domains. The substrate binding sequence combines by nucleotide complementarity and, possibly, non-hydrogen bond interactions with its TARGET sequence. The catalytic portion cleaves the TARGET RNA at a specific site. The substrate domain of a ribozyme can be engineered to direct it to a specified mRNA sequence. The ribozyme recognizes and then binds a TARGET mRNA through complementary base pairing. Once it is bound to the correct TARGET site, the ribozyme acts enzymatically to cut the TARGET mRNA. Cleavage of the mRNA by a ribozyme destroys its ability to direct synthesis of the corresponding polypeptide. Once the ribozyme has cleaved its TARGET sequence, it is released and can repeatedly bind and cleave at other mRNAs.

Exemplary ribozyme forms include a hammerhead motif, a hairpin motif, a hepatitis delta virus, group I intron or RNaseP RNA (in association with an RNA guide sequence) motif or Neurospora VS RNA motif Ribozymes possessing a hammerhead or hairpin structure are readily prepared since these catalytic RNA molecules can be expressed within cells from eukaryotic promoters (Chen, et al. (1992) Nucleic Acids Res. 20:4581-9). A ribozyme of the present invention can be expressed in eukaryotic cells from the appropriate DNA vector. If desired, the activity of the ribozyme may be augmented by its release from the primary transcript by a second ribozyme (Ventura, et al. (1993) Nucleic Acids Res. 21:3249-55).

Ribozymes may be chemically synthesized by combining an oligodeoxyribonucleotide with a ribozyme catalytic domain (20 nucleotides) flanked by sequences that hybridize to the TARGET mRNA after transcription. The oligodeoxyribonucleotide is amplified by using the substrate binding sequences as primers. The amplification product is cloned into a eukaryotic expression vector.

Ribozymes are expressed from transcription units inserted into DNA, RNA, or viral vectors. Transcription of the ribozyme sequences are driven from a promoter for eukaryotic RNA polymerase I (pol (I), RNA polymerase II (pol II), or RNA polymerase III (pol III). Transcripts from pol II or pol III promoters will be expressed at high levels in all cells; the levels of a given pol II promoter in a given cell type will depend on nearby gene regulatory sequences. Prokaryotic RNA polymerase promoters are also used, providing that the prokaryotic RNA polymerase enzyme is expressed in the appropriate cells (Gao and Huang, (1993) Nucleic Acids Res. 21:2867-72). It has been demonstrated that ribozymes expressed from these promoters can function in mammalian cells (Kashani-Sabet, et al. (1992) Antisense Res. Dev. 2:3-15).

In a particular embodiment the methods of the invention might be practiced using antisense polynucleotide, siRNA or shRNA comprising an antisense strand of 17-25 nucleotides complementary to a sense strand, wherein said sense strand is selected from 17-25 continuous nucleotides of a TARGET polynucleotide.

A particular inhibitory agent is a small interfering RNA (siRNA, particularly small hairpin RNA, “shRNA”). siRNA, particularly shRNA, mediate the post-transcriptional process of gene silencing by double stranded RNA (dsRNA) that is homologous in sequence to the silenced RNA. siRNA according to the present invention comprises a sense strand of 15-30, particularly 17-30, most particularly 17-25 nucleotides complementary or homologous to a contiguous 17-25 nucleotide sequence selected from the group of sequences described in SEQ ID NO: 1-17, particularly from the group of sequences described in SEQ ID NOs: 46-75, and an antisense strand of 15-30, particularly 17-30, most particularly 17-25, more specifically 19-21 nucleotides complementary to the sense strand. More particular siRNA according to the present invention comprises a sense strand selected from the group of sequences comprising SEQ ID NOs: 46-75. The most particular siRNA comprises sense and anti-sense strands that are 100 percent complementary to each other and the TARGET polynucleotide sequence. Particularly the siRNA further comprises a loop region linking the sense and the antisense strand.

A self-complementing single stranded shRNA molecule polynucleotide according to the present invention comprises a sense portion and an antisense portion connected by a loop region linker. Particularly, the loop region sequence is 4-30 nucleotides long, more particularly 5-15 nucleotides long and most particularly 8 or 12 nucleotides long. In a most particular embodiment the linker sequence is UUGCUAUA or GUUUGCUAUAAC (SEQ ID NO: 76). Self-complementary single stranded siRNAs form hairpin loops and are more stable than ordinary dsRNA. In addition, they are more easily produced from vectors.

Analogous to antisense RNA, the siRNA can be modified to confirm resistance to nucleolytic degradation, or to enhance activity, or to enhance cellular distribution, or to enhance cellular uptake, such modifications may consist of modified internucleoside linkages, modified nucleic acid bases, modified sugars and/or chemical linkage the siRNA to one or more moieties or conjugates. The nucleotide sequences are selected according to siRNA designing rules that give an improved reduction of the TARGET sequences compared to nucleotide sequences that do not comply with these siRNA designing rules (For a discussion of these rules and examples of the preparation of siRNA, WO 2004/094636 and US 2003/0198627, are hereby incorporated by reference).

Particular inhibitory agents include MicroRNAs (referred to as “miRNAs”). miRNA are small non-coding RNAs, belonging to a class of regulatory molecules found in many eukaryotic species that control gene expression by binding to complementary sites on target messenger RNA (mRNA) transcripts.

In vivo miRNAs are generated from larger RNA precursors (termed pri-miRNAs) that are processed in the nucleus into approximately 70 nucleotide pre-miRNAs, which fold into imperfect stem-loop structures. The pre-miRNAs undergo an additional processing step within the cytoplasm where mature miRNAs of 18-25 nucleotides in length are excised from one side of the pre-miRNA hairpin by an RNase III enzyme.

miRNAs have been shown to regulate gene expression in two ways. First, miRNAs binding to protein-coding mRNA sequences that are exactly complementary to the miRNA induce the RNA-mediated interference (RNAi) pathway. Messenger RNA targets are cleaved by ribonucleases in the RISC complex. In the second mechanism, miRNAs that bind to imperfect complementary sites on messenger RNA transcripts direct gene regulation at the posttranscriptional level but do not cleave their mRNA targets. miRNAs identified in both plants and animals use this mechanism to exert translational control over their gene targets.

### Low Molecular Weight Compounds

Particular drug candidate compounds are low molecular weight compounds. Low molecular weight compounds, for example with a molecular weight of 500 Dalton or less, are likely to have good absorption and permeation in biological systems and are consequently more likely to be successful drug candidates than compounds with a molecular weight above 500 Dalton (Lipinski et al., 2001)). Peptides comprise another particular class of drug candidate compounds. Peptides may be excellent drug candidates and there are multiple examples of commercially valuable peptides such as fertility hormones and platelet aggregation inhibitors. Natural compounds are another particular class of drug candidate compound. Such compounds are found in and extracted from natural sources, and which may thereafter be synthesized. The lipids are another particular class of drug candidate compound.

### Antibodies

Another preferred class of drug candidate compounds is an antibody. The present invention also provides antibodies directed against the TARGETS. These antibodies may be endogenously produced to bind to the TARGETS within the cell, or added to the tissue to bind to the TARGET polypeptide present outside the cell. These antibodies may be monoclonal antibodies or polyclonal antibodies. The present invention includes chimeric, single chain, and humanized antibodies, as well as FAb fragments and the products of a FAb expression library, and Fv fragments and the products of an Fv expression library.

In certain embodiments, polyclonal antibodies may be used in the practice of the invention. The skilled artisan knows methods of preparing polyclonal antibodies. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. Antibodies may also be generated against the intact TARGET protein or polypeptide, or against a fragment, derivatives including conjugates, or other epitope of the TARGET protein or polypeptide, such as the TARGET embedded in a cellular membrane, or a library of antibody variable regions, such as a phage display library.

It may be useful to conjugate the immunizing agent to a protein known to be immunogenic in the mammal being immunized Examples of such immunogenic proteins include but are not limited to keyhole limpet hemocyanin, serum albumin, bovine thyroglobulin, and soybean trypsin inhibitor. Examples of adjuvants that may be employed include Freund's complete adjuvant and MPL-TDM adjuvant (monophosphoryl Lipid A, synthetic trehalose dicorynomycolate). One skilled in the art without undue experimentation may select the immunization protocol.

In some embodiments, the antibodies may be monoclonal antibodies. Monoclonal antibodies may be prepared using methods known in the art. The monoclonal antibodies of the present invention may be “humanized” to prevent the host from mounting an immune response to the antibodies. A “humanized antibody” is one in which the complementarity determining regions (CDRs) and/or other portions of the light and/or heavy variable domain framework are derived from a non-human immunoglobulin, but the remaining portions of the molecule are derived from one or more human immunoglobulins. Humanized antibodies also include antibodies characterized by a humanized heavy chain associated with a donor or acceptor unmodified light chain or a chimeric light chain, or vice versa. The humanization of antibodies may be accomplished by methods known in the art (see, e.g. Mark and Padlan, (1994) “Chapter 4. Humanization of Monoclonal Antibodies”, The Handbook of Experimental Pharmacology Vol. 113, Springer-Verlag, New York). Transgenic animals may be used to express humanized antibodies.

Human antibodies can also be produced using various techniques known in the art, including phage display libraries (Hoogenboom and Winter, (1991) J. Mol. Biol. 227:381-8; Marks et al. (1991). J. Mol. Biol. 222:581-97). The techniques of Cole, et al. and Boerner, et al. are also available for the preparation of human monoclonal antibodies (Cole, et al. (1985) Monoclonal Antibodies and Cancer Therapy, Alan R. Liss, p. 77; Boerner, et al (1991). J. Immunol., 147(1):86-95).

Techniques known in the art for the production of single chain antibodies can be adapted to produce single chain antibodies to the TARGETS. The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain cross-linking. Alternatively; the relevant cysteine residues are substituted with another amino acid residue or are deleted so as to prevent cross-linking.

Bispecific antibodies are monoclonal, preferably human or humanized, antibodies that have binding specificities for at least two different antigens and preferably for a cell-surface protein or receptor or receptor subunit. In the present case, one of the binding specificities is for one domain of the TARGET; the other one is for another domain of the TARGET.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, (1983) Nature 305:537-9). Because of the random assortment of immunoglobulin heavy and light chains, these hybridomas (quadromas) produce a potential mixture of ten different antibody molecules, of which only one has the correct bispecific structure. Affinity chromatography steps usually accomplish the purification of the correct molecule. Similar procedures are disclosed in Trauneeker, et al. (1991) EMBO J. 10:3655-9.

A special aspect of the methods of the present invention relates to the down-regulation or blocking of the expression of a TARGET polypeptide by the induced expression of a polynucleotide encoding an intracellular binding protein that is capable of selectively interacting with the TARGET polypeptide. An intracellular binding protein includes an activity-inhibitory agent and any protein capable of selectively interacting, or binding, with the polypeptide in the cell in which it is expressed and neutralizing the function of the polypeptide. Particularly, the intracellular binding protein may be an antibody, particularly a neutralizing antibody, or a fragment of an antibody or neutralizing antibody having binding affinity to an epitope of the TARGET polypeptide of SEQ ID NO: 18-34. More particularly, the intracellular binding protein is a single chain antibody.

### Pharmaceutical Compositions, Related Uses and Methods

The antibodies or a fragments thereof which specifically bind to a TARGET polypeptide and expression inhibiting agents selected from the group consisting of an antisense polynucleotide, a ribozyme, a small interfering RNA (siRNA), microRNA (miRNA) and a short-hairpin RNA (shRNA) may be used as therapeutic agents for the treatment of conditions in mammals that are causally related or attributable to EMT.

The present invention relates to pharmaceutical compositions comprising an antibody or a fragment thereof which specifically binds to a TARGET polypeptide, for use in the treatment of a disease associated with EMT. In a particular aspect, the present invention provides a method of treating a mammal having, or at risk of having a fibrotic disease or cancer.

In particular aspect, the present invention provides a method of treating a mammal having, or at risk of having a disease associated with EMT, said method comprising administering an effective condition-treating or condition-preventing amount of one or more of the pharmaceutical compositions comprising an antibody or a fragment thereof which specifically binds to a TARGET polypeptide. In a particular aspect, the present invention provides a method of treating a mammal having, or at risk of having a fibrotic disease or cancer. In specific embodiment, said antibody is a monoclonal antibody. In alternative embodiment said antibody is a single chain antibody. In particular embodiment said disease is a carcinoma.

In another aspect the present invention provides an antibody or a fragment thereof which specifically binds to a TARGET polypeptide for use in the treatment, and/or prophylaxis of a disease associated with EMT. In a specific embodiment, said disease is selected from a fibrotic disease or cancer. In specific embodiment, said antibody is a monoclonal antibody. In alternative embodiment said antibody is a single chain antibody. In particular embodiment said disease is a carcinoma.

In yet another aspect, the present invention provides an antibody or a fragment thereof which specifically binds to a TARGET polypeptide, or a pharmaceutical composition comprising an antibody or a fragment thereof which specifically binds to a TARGET polypeptide for use in the manufacture of a medicament for the treatment, or prophylaxis of a disease associated with EMT. In a specific embodiment, said condition is selected from a fibrotic disease or cancer. In specific embodiment, said antibody is a monoclonal antibody. In alternative embodiment said antibody is a single chain antibody. In particular embodiment said disease is a carcinoma.

A particular regimen of the present method comprises the administration to a subject suffering from a disease associated with EMT, of an effective amount of an antibody or a fragment thereof which specifically binds to a TARGET polypeptide for a period of time sufficient to reduce the level of EMT in the subject, and preferably terminate the processes responsible for said condition. A special embodiment of the method comprises administering of an effective amount of an antibody or a fragment thereof which specifically binds to a TARGET polypeptide to a subject patient suffering from or susceptible to the development of a fibrotic disease, for a period of time sufficient to reduce or prevent, respectively, disease associated with EMT in said patient, and preferably terminate, the processes responsible for said condition. In specific embodiment, said antibody is a monoclonal antibody. In alternative embodiment said antibody is a single chain antibody. In particular embodiment said condition is a fibrotic disease or cancer.

The present invention further relates to compositions comprising an agent is selected from the group consisting of an antisense polynucleotide, a ribozyme, a small interfering RNA (siRNA), microRNA (miRNA), and a short-hairpin RNA (shRNA), wherein said agent comprises a nucleic acid sequence complementary to, or engineered from, a naturally-occurring polynucleotide sequence of about 17 to about 30 contiguous nucleotides of a nucleic acid sequence selected from the group consisting of SEQ ID NO: 1-17. These agents are, otherwise, referred herein to as expression inhibitory agents.

In particular aspect, the present invention provides a method of treating a mammal having, or at risk of having a disease associated with EMT, said method comprising administering an effective condition-treating or condition-preventing amount of one or more of the pharmaceutical compositions comprising said expression inhibitory agent. In a particular aspect, the present invention provides a method of treating a mammal having, or at risk of having a fibrotic disease or cancer.

In another aspect the present invention provides expression inhibitory agents for use in the treatment, and/or prophylaxis of a disease associated with EMT. In a specific embodiment, said disease is selected from a fibrotic disease or cancer. In particular embodiment said condition is a carcinoma.

In yet another aspect, the present invention provides expression inhibitory agents, or a pharmaceutical composition comprising said expression inhibitory agents for use in the manufacture of a medicament for the treatment, or prophylaxis of a disease associated with EMT. In a specific embodiment, said disease is selected from a fibrotic disease or cancer.

A particular regimen of the present method comprises the administration to a subject suffering from a disease associated with EMT, of an effective amount of an expression inhibitory agent for a period of time sufficient to reduce the level of EMT, and preferably terminate the processes responsible for said disease. A special embodiment of the method comprises administering of an effective amount of an antibody or a fragment thereof which specifically binds to a TARGET polypeptide to a subject patient suffering from or susceptible to the development of a disease associated with EMT, for a period of time sufficient to reduce or prevent, respectively, EMT in said patient, and preferably terminate, the processes of EMT responsible for said disease. In particular embodiment said disease is a fibrotic disease or cancer.

In a particular aspect, said fibrotic disease is selected from idiopathic pulmonary fibrosis (IPF), cystic fibrosis, other diffuse parenchymal lung diseases of different etiologies including iatrogenic drug-induced fibrosis, occupational and/or environmental induced fibrosis, granulomatous diseases (sarcoidosis, hypersensitivity pneumonia), collagen vascular disease, alveolar proteinosis, langerhans cell granulomatosis, lymphangioleiomyomatosis, inherited diseases (Hermansky-Pudlak Syndrome, tuberous sclerosis, neurofibromatosis, metabolic storage disorders, familial interstitial lung disease), radiation induced fibrosis, chronic obstructive pulmonary disease (COPD), scleroderma, bleomycin induced pulmonary fibrosis, chronic asthma, silicosis, asbestos induced pulmonary fibrosis, acute respiratory distress syndrome (ARDS), kidney fibrosis, tubulointerstitium fibrosis, glomerular nephritis, focal segmental glomerular sclerosis, IgA nephropathy, hypertension, Alport syndrome, gut fibrosis, liver fibrosis, cirrhosis, alcohol induced liver fibrosis, toxic/drug induced liver fibrosis, hemochromatosis, nonalcoholic steatohepatitis (NASH), biliary duct injury, primary biliary cirrhosis, infection induced liver fibrosis, viral induced liver fibrosis, autoimmune hepatitis, corneal scarring, hypertrophic scarring, Dupuytren disease, keloids, cutaneous fibrosis, cutaneous scleroderma, systemic sclerosis, spinal cord injury/fibrosis, myelofibrosis, vascular restenosis, atherosclerosis, arteriosclerosis, Wegener's granulomatosis and Peyronie's disease.

In another aspect, said cancer is selected from melanoma, lymphoma, leukaemia, fibrosarcoma, rhabdomyosarcoma, mastocytoma, colorectal cancer, prostate cancer, small cell lung cancer and non-small cell lung cancer, breast cancer, pancreatic cancer, bladder cancer, renal cancer, gastric cancer, glioblastoma, primary liver cancer, ovarian cancer, prostate cancer and uterine leiomyosarcoma. In a more specific aspect. In more specific aspect said cancer is a cancer associated and/or correlated with EMT, more particular cancer metastasis.

Another aspect of the present invention relates to compositions, comprising a DNA expression vector capable of expressing a polynucleotide capable of inhibition of expression of a TARGET polypeptide and described as an expression inhibitory agent.

The present invention provides compounds, compositions, and methods useful for modulating the expression of the TARGET genes, specifically those TARGET genes associated with EMT and for treating such conditions by RNA interference (RNAi) using small nucleic acid molecules. In particular, the instant invention features small nucleic acid molecules, i.e., short interfering nucleic acid (siNA) molecules including, but not limited to, short interfering RNA (siRNA), double-stranded RNA (dsRNA), micro-RNA (miRNA), short hairpin RNA (shRNA) and circular RNA molecules and methods used to modulate the expression of the TARGET genes and/or other genes involved in pathways of the TARGET gene expression and/or activity.

A particular aspect of these compositions and methods relates to the down-regulation or blocking of the expression of the TARGET by the induced expression of a polynucleotide encoding an intracellular binding protein that is capable of selectively interacting with the TARGET. An intracellular binding protein includes any protein capable of selectively interacting, or binding, with the polypeptide in the cell in which it is expressed and neutralizing the function of the polypeptide. Preferably, the intracellular binding protein is a neutralizing antibody or a fragment of a neutralizing antibody having binding affinity to an epitope of a TARGET selected from the group consisting of SEQ ID NO: 18-34. More preferably, the intracellular binding protein is a single chain antibody.

Antibodies according to the invention may be delivered as a bolus only, infused over time or both administered as a bolus and infused over time. Those skilled in the art may employ different formulations for polynucleotides than for proteins. Similarly, delivery of polynucleotides or polypeptides will be specific to particular cells, conditions, locations, etc.

A particular embodiment of this composition comprises the expression-inhibiting agent selected from the group consisting of antisense RNA, antisense oligodeoxynucleotide (ODN), a ribozyme that cleaves the polyribonucleotide coding for a TARGET selected from the group consisting of SEQ ID NO: 1-17, a small interfering RNA (siRNA), and a microRNA that is sufficiently homologous to a portion of the polyribonucleotide coding for a TARGET selected from the group consisting of SEQ ID NO: 1-17, such that the siRNA or microRNA interferes with the translation of the TARGET polyribonucleotide to the TARGET polypeptide.

The polynucleotide expressing the expression-inhibiting agent, or a polynucleotide expressing the TARGET polypeptide in cells, is particularly included within a vector. The polynucleic acid is operably linked to signals enabling expression of the nucleic acid sequence and is introduced into a cell utilizing, preferably, recombinant vector constructs, which will express the antisense nucleic acid once the vector is introduced into the cell. A variety of viral-based systems are available, including adenoviral, retroviral, adeno-associated viral, lentiviral, herpes simplex viral or a sendaiviral vector systems, and all may be used to introduce and express polynucleotide sequence for the expression-inhibiting agents or the polynucleotide expressing the TARGET polypeptide in the target cells.

Particularly, the viral vectors used in the methods of the present invention are replication defective. Such replication defective vectors will usually pack at least one region that is necessary for the replication of the virus in the infected cell. These regions can either be eliminated (in whole or in part), or be rendered non-functional by any technique known to a person skilled in the art. These techniques include the total removal, substitution, partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome, which are necessary for encapsidating, the viral particles.

In a preferred embodiment, the viral element is derived from an adenovirus. Preferably, the vehicle includes an adenoviral vector packaged into an adenoviral capsid, or a functional part, derivative, and/or analogue thereof. Adenovirus biology is also comparatively well-known on the molecular level. Many tools for adenoviral vectors have been and continue to be developed, thus making an adenoviral capsid a preferred vehicle for incorporating in a library of the invention. An adenovirus is capable of infecting a wide variety of cells. However, different adenoviral serotypes have different preferences for cells. To combine and widen the target cell population that an adenoviral capsid of the invention can enter in a preferred embodiment, the vehicle includes adenoviral fiber proteins from at least two adenoviruses. Preferred adenoviral fiber protein sequences are serotype 17, 45 and 51. Techniques or construction and expression of these chimeric vectors are disclosed in US 2003/0180258 and US 2004/0071660, hereby incorporated by reference.

In a preferred embodiment, the nucleic acid derived from an adenovirus includes the nucleic acid encoding an adenoviral late protein or a functional part, derivative, and/or analogue thereof. An adenoviral late protein, for instance an adenoviral fiber protein, may be favorably used to target the vehicle to a certain cell or to induce enhanced delivery of the vehicle to the cell. Preferably, the nucleic acid derived from an adenovirus encodes for essentially all adenoviral late proteins, enabling the formation of entire adenoviral capsids or functional parts, analogues, and/or derivatives thereof. Preferably, the nucleic acid derived from an adenovirus includes the nucleic acid encoding adenovirus E2A or a functional part, derivative, and/or analogue thereof. Preferably, the nucleic acid derived from an adenovirus includes the nucleic acid encoding at least one E4-region protein or a functional part, derivative, and/or analogue thereof, which facilitates, at least in part, replication of an adenoviral derived nucleic acid in a cell. The adenoviral vectors used in the examples of this application are exemplary of the vectors useful in the present method of treatment invention.

Certain embodiments of the present invention use retroviral vector systems. Retroviruses are integrating viruses that infect dividing cells, and their construction is known in the art. Retroviral vectors can be constructed from different types of retrovirus, such as, MoMuLV (“murine Moloney leukemia virus”) MSV (“murine Moloney sarcoma virus”), HaSV (“Harvey sarcoma virus”); SNV (“spleen necrosis virus”); RSV (“Rous sarcoma virus”) and Friend virus. Lentiviral vector systems may also be used in the practice of the present invention.

In other embodiments of the present invention, adeno-associated viruses (“AAV”) are utilized. The AAV viruses are DNA viruses of relatively small size that integrate, in a stable and site-specific manner, into the genome of the infected cells. They are able to infect a wide spectrum of cells without inducing any effects on cellular growth, morphology or differentiation, and they do not appear to be involved in human pathologies.

As discussed hereinabove, recombinant viruses may be used to introduce DNA encoding polynucleotide agents useful in the present invention. Recombinant viruses according to the invention are generally formulated and administered in the form of doses of between about 104 and about 1014 pfu. In the case of AAVs and adenoviruses, doses of from about 106 to about 1011 pfu are particularly used. The term pfu (“plaque-forming unit”) corresponds to the infective power of a suspension of virions and is determined by infecting an appropriate cell culture and measuring the number of plaques formed. The techniques for determining the pfu titre of a viral solution are well documented in the prior art.

In the vector construction, the polynucleotide agents of the present invention may be linked to one or more regulatory regions. Selection of the appropriate regulatory region or regions is a routine matter, within the level of ordinary skill in the art. Regulatory regions include promoters, and may include enhancers, suppressors, etc.

Promoters that may be used in the expression vectors of the present invention include both constitutive promoters and regulated (inducible) promoters. The promoters may be prokaryotic or eukaryotic depending on the host. Among the prokaryotic (including bacteriophage) promoters useful for practice of this invention are lac, lacZ, T3, T7, lambda Pᵣ, Pₗ, and trp promoters. Among the eukaryotic (including viral) promoters useful for practice of this invention are ubiquitous promoters (e.g. HPRT, vimentin, actin, tubulin), therapeutic gene promoters (e.g. MDR type, CFTR, factor VIII), tissue-specific promoters, including animal transcriptional control regions, which exhibit tissue specificity and have been utilized in transgenic animals, e.g. chymase gene control region which is active in mast cells (Liao et al., (1997), Journal of Biological Chemistry, 272: 2969-2976), immunoglobulin gene control region which is active in lymphoid cells (Grosschedl, et al. (1984) Cell 38:647-58; Adames, et al. (1985) Nature 318:533-8; Alexander, et al. (1987) Mol. Cell. Biol. 7:1436-44), mouse mammary tumor virus control region which is active in testicular, breast, lymphoid and mast cells (Leder, et al. (1986) Cell 45:485-95), beta-globin gene control region which is active in myeloid cells (Mogram, et al. (1985) Nature 315:338-40; Kollias, et al. (1986) Cell 46:89-94), the CMV promoter and the Visna LTR (Sidiropoulos et al., (2001), Gene Therapy, 8:223-231)

Other promoters which may be used in the practice of the invention include promoters which are preferentially activated in dividing cells, promoters which respond to a stimulus (e.g. steroid hormone receptor, retinoic acid receptor), tetracycline-regulated transcriptional modulators, cytomegalovirus immediate-early, retroviral LTR, metallothionein, SV-40, E1a, and MLP promoters. Further promoters which may be of use in the practice of the invention include promoters which are active and/or expressed in macrophages or other cell types contributing to inflammation such as dendritic cells, monocytes, neutrophils, mast cells, endothelial cells, epithelial cells, muscle cells, etc.

Additional vector systems include the non-viral systems that facilitate introduction of polynucleotide agents into a patient. For example, a DNA vector encoding a desired sequence can be introduced in vivo by lipofection. Synthetic cationic lipids designed to limit the difficulties encountered with liposome-mediated transfection can be used to prepare liposomes for in vivo transfection of a gene encoding a marker (Feigner, et. al. (1987) Proc. Natl. Acad Sci. USA 84:7413-7); see Mackey, et al. (1988) Proc. Natl. Acad. Sci. USA 85:8027-31; Ulmer, et al. (1993) Science 259:1745-8). The use of cationic lipids may promote encapsulation of negatively charged nucleic acids, and also promote fusion with negatively charged cell membranes (Feigner and Ringoid, (1989) Nature 337:387-8). Particularly useful lipid compounds and compositions for transfer of nucleic acids are described in International Patent Publications WO 95/18863 and WO 96/17823, and in U.S. Pat. No. 5,459,127. The use of lipofection to introduce exogenous genes into the specific organs in vivo has certain practical advantages and directing transfection to particular cell types would be particularly advantageous in a tissue with cellular heterogeneity, for example, pancreas, liver, kidney, and the brain. Lipids may be chemically coupled to other molecules for the purpose of targeting. Targeted peptides, e.g., hormones or neurotransmitters, and proteins for example, antibodies, or non-peptide molecules could be coupled to liposomes chemically. Other molecules are also useful for facilitating transfection of a nucleic acid in vivo, for example, a cationic oligopeptide (e.g., International Patent Publication WO 95/21931), peptides derived from DNA binding proteins (e.g., International Patent Publication WO 96/25508), or a cationic polymer (e.g., International Patent Publication WO 95/21931).

It is also possible to introduce a DNA vector in vivo as a naked DNA plasmid (see U.S. Pat. Nos. 5,693,622, 5,589,466 and 5,580,859). Naked DNA vectors for therapeutic purposes can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wilson, et al. (1992) J. Biol. Chem. 267:963-7; Wu and Wu, (1988) J. Biol. Chem. 263:14621-4; Hartmut, et al. Canadian Patent Application No. 2,012,311, filed Mar. 15, 1990; Williams, et al (1991). Proc. Natl. Acad. Sci. USA 88:2726-30). Receptor-mediated DNA delivery approaches can also be used (Curiel, et al. (1992) Hum. Gene Ther. 3:147-54; Wu and Wu, (1987) J. Biol. Chem. 262:4429-32).

A biologically compatible composition is a composition, that may be solid, liquid, gel, or other form, in which the compound, polynucleotide, vector, and antibody of the invention is maintained in an active form, e.g., in a form able to effect a biological activity. For example, a compound of the invention would have inverse agonist or antagonist activity on the TARGET; a nucleic acid would be able to replicate, translate a message, or hybridize to a complementary mRNA of the TARGET; a vector would be able to transfect a target cell and express the antisense, antibody, ribozyme or siRNA as described hereinabove; an antibody would bind a the TARGET polypeptide domain.

A particular biologically compatible composition is an aqueous solution that is buffered using, e.g., Tris, phosphate, or HEPES buffer, containing salt ions. Usually the concentration of salt ions will be similar to physiological levels. Biologically compatible solutions may include stabilizing agents and preservatives. In a more preferred embodiment, the biocompatible composition is a pharmaceutically acceptable composition. Such compositions can be formulated for administration by topical, oral, parenteral, intranasal, subcutaneous, and intraocular, routes. Parenteral administration is meant to include intravenous injection, intramuscular injection, intraarterial injection or infusion techniques. The composition may be administered parenterally in dosage unit formulations containing standard, well-known non-toxic physiologically acceptable carriers, adjuvants and vehicles as desired.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical compositions for oral use can be prepared by combining active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethyl-cellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate. Dragee cores may be used in conjunction with suitable coatings, such as concentrated sugar solutions, which may also contain gum arabic, talc, polyvinyl-pyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Particular sterile injectable preparations can be a solution or suspension in a non-toxic parenterally acceptable solvent or diluent. Examples of pharmaceutically acceptable carriers are saline, buffered saline, isotonic saline (for example, monosodium or disodium phosphate, sodium, potassium; calcium or magnesium chloride, or mixtures of such salts), Ringer's solution, dextrose, water, sterile water, glycerol, ethanol, and combinations thereof 1,3-butanediol and sterile fixed oils are conveniently employed as solvents or suspending media. Any bland fixed oil can be employed including synthetic mono- or di-glycerides. Fatty acids such as oleic acid also find use in the preparation of injectables.

The compounds or compositions of the invention may be combined for administration with or embedded in polymeric carrier(s), biodegradable or biomimetic matrices or in a scaffold. The carrier, matrix or scaffold may be of any material that will allow composition to be incorporated and expressed and will be compatible with the addition of cells or in the presence of cells. Particularly, the carrier matrix or scaffold is predominantly non-immunogenic and is biodegradable. Examples of biodegradable materials include, but are not limited to, polyglycolic acid (PGA), polylactic acid (PLA), hyaluronic acid, catgut suture material, gelatin, cellulose, nitrocellulose, collagen, albumin, fibrin, alginate, cotton, or other naturally-occurring biodegradable materials. It may be preferable to sterilize the matrix or scaffold material prior to administration or implantation, e.g., by treatment with ethylene oxide or by gamma irradiation or irradiation with an electron beam. In addition, a number of other materials may be used to form the scaffold or framework structure, including but not limited to: nylon (polyamides), dacron (polyesters), polystyrene, polypropylene, polyacrylates, polyvinyl compounds (e.g., polyvinylchloride), polycarbonate (PVC), polytetrafluorethylene (PTFE, teflon), thermanox (TPX), polymers of hydroxy acids such as polylactic acid (PLA), polyglycolic acid (PGA), and polylactic acid-glycolic acid (PLGA), polyorthoesters, polyanhydrides, polyphosphazenes, and a variety of polyhydroxyalkanoates, and combinations thereof. Matrices suitable include a polymeric mesh or sponge and a polymeric hydrogel. In the particular embodiment, the matrix is biodegradable over a time period of less than a year, more particularly less than six months, most particularly over two to ten weeks. The polymer composition, as well as method of manufacture, can be used to determine the rate of degradation. For example, mixing increasing amounts of polylactic acid with polyglycolic acid decreases the degradation time. Meshes of polyglycolic acid that can be used can be obtained commercially, for instance, from surgical supply companies (e.g., Ethicon, N.J). In general, these polymers are at least partially soluble in aqueous solutions, such as water, buffered salt solutions, or aqueous alcohol solutions, that have charged side groups, or a monovalent ionic salt thereof.

The composition medium can also be a hydrogel, which is prepared from any biocompatible or non-cytotoxic homo- or hetero-polymer, such as a hydrophilic polyacrylic acid polymer that can act as a drug absorbing sponge. Certain of them, such as, in particular, those obtained from ethylene and/or propylene oxide are commercially available. A hydrogel can be deposited directly onto the surface of the tissue to be treated, for example during surgical intervention.

Embodiments of pharmaceutical compositions of the present invention comprise a replication defective recombinant viral vector encoding the agent of the present invention and a transfection enhancer, such as poloxamer. An example of a poloxamer is Poloxamer 407, which is commercially available (BASF, Parsippany, N.J.) and is a non-toxic, biocompatible polyol. A poloxamer impregnated with recombinant viruses may be deposited directly on the surface of the tissue to be treated, for example during a surgical intervention. Poloxamer possesses essentially the same advantages as hydrogel while having a lower viscosity.

The active agents may also be entrapped in microcapsules prepared, for example, by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (1980) 16th edition, Osol, A. Ed.

Sustained-release preparations may be prepared. Suitable examples of sustained-release preparations include semi-permeable matrices of solid hydrophobic polymers containing the antibody, which matrices are in the form of shaped articles, for example, films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™. (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

As used herein, therapeutically effective dose means that amount of protein, polynucleotide, peptide, or its antibodies, agonists or antagonists, which ameliorate the symptoms or condition. Therapeutic efficacy and toxicity of such compounds can be determined by standard pharmaceutical procedures in cell cultures or experimental animals, for example, ED₅₀ (the dose therapeutically effective in 50% of the population) and LD₅₀ (the dose lethal to 50% of the population). The dose ratio of toxic to therapeutic effects is the therapeutic index, and it can be expressed as the ratio, LD₅₀/ED₅₀. Pharmaceutical compositions that exhibit large therapeutic indices are particular. The data obtained from cell culture assays and animal studies are used in formulating a range of dosage for human use. The dosage of such compounds lies particularly within a range of circulating concentrations that include the ED₅₀ with little or no toxicity. The dosage varies within this range depending upon the dosage form employed, sensitivity of the patient, and the route of administration.

For any compound, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. The exact dosage is chosen by the individual physician in view of the patient to be treated. Dosage and administration are adjusted to provide sufficient levels of the active moiety or to maintain the desired effect. Additional factors which may be taken into account include the severity of the disease state, age, weight and gender of the patient; diet, desired duration of treatment, method of administration, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

The pharmaceutical compositions according to this invention may be administered to a subject by a variety of methods. They may be added directly to targeted tissues, complexed with cationic lipids, packaged within liposomes, or delivered to targeted cells by other methods known in the art. Localized administration to the desired tissues may be done by direct injection, transdermal absorption, catheter, infusion pump or stent. The DNA, DNA/vehicle complexes, or the recombinant virus particles are locally administered to the site of treatment. Alternative routes of delivery include, but are not limited to, intravenous injection, intramuscular injection, subcutaneous injection, aerosol inhalation, oral (tablet or pill form), topical, systemic, ocular, intraperitoneal and/or intrathecal delivery. Examples of ribozyme delivery and administration are provided in Sullivan et al. WO 94/02595.

Administration of an expression-inhibiting agent or an antibody of the present invention to the subject patient includes both self-administration and administration by another person. The patient may be in need of treatment for an existing disease or medical condition, or may desire prophylactic treatment to prevent or reduce the risk for diseases and medical conditions affected by differentiation of macrophages into alternatively-activated macrophages. The expression-inhibiting agent of the present invention may be delivered to the subject patient orally, transdermally, via inhalation, injection, nasally, rectally or via a sustained release formulation.

### In Vitro Methods

The present invention also provides an in vitro method of inhibiting EMT, said method comprising contacting a population of epithelial cells with an inhibitor of the activity or expression of a TARGET polypeptide. In a particular embodiment said inhibitor is an antibody. In an alternative embodiment said antibody is a monoclonal antibody.

The present invention further relates to an in vitro method of inhibiting EMT, said method comprising contacting a population of epithelial cells with an inhibitor selected from the group consisting of an antisense polynucleotide, a ribozyme, a small interfering RNA (siRNA), mi croRNA (miRNA) and a short-hairpin RNA (shRNA), wherein said inhibitor comprises a nucleic acid sequence complementary to, or engineered from, a naturally-occurring polynucleotide sequence of about 17 to about 30 contiguous nucleotides of a nucleic acid encoding a TARGET polypeptide.

The down regulation of gene expression using antisense nucleic acids can be achieved at the translational or transcriptional level. Antisense nucleic acids of the invention are particularly nucleic acid fragments capable of specifically hybridizing with all or part of a nucleic acid encoding a TARGET polypeptide or the corresponding messenger RNA. In addition, antisense nucleic acids may be designed which decrease expression of the nucleic acid sequence capable of encoding a TARGET polypeptide by inhibiting splicing of its primary transcript. Any length of antisense sequence is suitable for practice of the invention so long as it is capable of down-regulating or blocking expression of a nucleic acid coding for a TARGET. Particularly, the antisense sequence is at least about 15-30, and particularly at least 17 nucleotides in length. The preparation and use of antisense nucleic acids, DNA encoding antisense RNAs and the use of oligo and genetic antisense is known in the art.

### EXAMPLES

The invention is further illustrated using examples provided below. It would be obvious to a person skilled in the art that the examples might be easily modified or adapted to particular types of conditions, scale or cell types using routine adaptations.

Example 1 describes the set-up of the EMT primary assay and the primary screen using said assay

Example 2 describes the re-screen of the hits from the primary screen of Example 1

Example 3 describes the EMT2 validation assay

Example 4 describes the “on target’ validation using additional shRNA constructs and toxicity assessment of shRNA constructs

Example 5 describes the ATPlite secondary toxicity assay used to validate identified hits

Example 6 describes the whole transcriptome sequencing in HBEC

### Example 1

### EMT Assay Primary Screen

### 1.1 Background

Airway remodeling and fibrosis are important features in the pathogenesis of fibrosis. Epithelial mesenchymal transition (EMT) has been proposed as a mechanism for an increase in number of fibroblast-like cells and collagen overproduction leading to fibrosis. Several studies have demonstrated that EMT may occur in human lung epithelial cell lines and primary bronchial epithelial cells upon exposure to TGF13. A special TGFβ-induced EMT assay was developed in primary Human Bronchial Epithelial Cells (HBEC) using several common markers of EMT.

1.2 Cell cultures and donors

HBEC were obtained from the Dept of Pulmonology (LUMC, Leiden, The Netherlands). HBEC were derived from lung resection tissue of patients undergoing surgery for lung tumors. Bronchial epithelial cells were isolated by protease digestion and cultured as previously described (van Wetering, 2000). Three donors were used throughout all experiments. For primary screen and on-target analysis donor Br299 was used, for rescreen donor Br291 and for target validation in a secondary assay donor Br282 was used. All three donors were COPD patients.

TABLE 2
Overview of donors used throughout examples

| Donor name | Type | Supplier | Cell passage | Used for |
|---|---|---|---|---|
| Br291 | HBEC-COPD | LUMC | 1 | Primary and on- |
| | | | | target screen |
| Br299 | HBEC-COPD | LUMC | 1 | Re-screen |
| Br282 | HBEC-COPD | LUMC | 1 | Validation |

### 1.3 FN and MMP10 Read-Outs

MMPs have the potential to cleave extracellular matrix (ECM) proteins. These proteins may also include collagens and other proteins such as fibronectin, proteins that are known to compose the scar tissue upon triggers causing fibrosis. Amongst the MMPs, MMP10 is involved in cleavage of ECM and hence was used as a read-out in the validation, representing the MMP-inducing fibrosis pathway of interest. MMP10 was tested by MSD. Increased levels of MMP10 are detected upon triggering with NTHi in both COPD and non-COPD donors.

FN and MMP10 were measured using the Mesoscal Discovery (MSD) platform on a SECTOR® Imager 6000 instrument (MSD). MMP10 was measured using a custom made assay from MSD (product number L211A-1, MSD) according to manufacturer's indications. FN was measured using in-house developed assay. Hereto, MSD 384-well standard plates (product number L21XA-4, MSD) were coated with anti-human FN1 capture antibody (product number AF1918, R&D Systems). Following addition of samples, a biotinylated anti-human FN1 detection antibody (product number BAF1918, R&D Systems) and subsequently SULFO-TAG-streptavidin (product number R32AD-5, MSD) were added. Further detection of signal was performed according to standard manufacturer's recommendations on the SECTOR® Imager 6000 instrument (MSD).

### 1.4 Triggers

Batches of UV irradiated non-typeable Haemophilus influenzae (NTHi) were generated. Bacteria were irradiated in aliquots of 2.9×10⁸/mL (NTHi) and stored at −80° C. until use. A combination of 0.5 ng/mL TGFβ-1, 5 ng/mL TNFα and 0.5×10⁷ UV-killed NTHi bacteria/mL was used to trigger cells

### 1.5 Positive and Negative Controls

Three negative controls targeting the firefly luciferase (ffluc_v19, ffluc_v21, ffluc_v24) and five positive control shRNA viruses (SMAD3_v3, SMAD4_v5 and v7, TGFβR1_v1 and TGFβR2_v7) were added to each library plate in column 7.

Table 3
An overview of controls used in the primary screen

| Control shRNA | Sequence | SEQ ID NO: |
|---|---|---|
| Ffluc_v19 | GAATCGATATTGTTACAAC | 35 |
| Ffluc_v21 | ATATCGAGGTGAACATCAC | 36 |
| Ffluc_v24 | GCAGTCAAGTTTCCACAAC | 37 |
| SMAD3_v3 | GCTCCATCTCCTACTACGA | 38 |
| SMAD4_v5 | GTGTTCCATTGCTTACTTT | 39 |
| SMAD4_v7 | GCAGAGTAATGCTCCATCA | 40 |
| TGFBR1_v1 | GAAAGCATTGGCAAAGGTC | 41 |
| TGFBR2_v7 | GCAGTCAAGTTTCCACAAC | 42 |

### 1.6 Statistical Acceptance Criteria

Acceptance criteria for primary screen source plates were the following:
Spearman correlation >0.4 or Kappa value >0.2
At least one of the positive controls used for primary screen with secreted fibronectin (FN) as read-out should have an IQR<−1.5 in duplicate
Two out of three positive controls used for primary screen with secreted fibronectin FN (P1, P2, P5) should give >40% inhibition as compared to the average of the negative control viruses
At least three of the positive controls used for primary screen with secreted MMP10 as read-out should have an IQR<−1.5 in duplicate.

Plates that did not fulfill these criteria were rescreened again.

### 1.7 Protocol

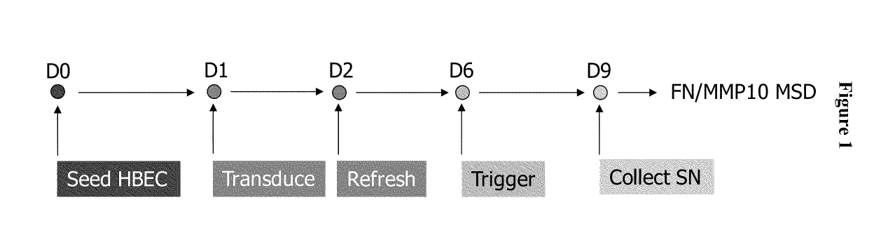

The adenoviral library, comprising more than 12,000 adenoviral shRNA constructs, was screened in the primary screen. The full screen consisted of 143×96-well plates and was performed in biological duplicate. A schematic overview of the EMT assay is presented in FIG. 1.

The primary screen was performed in six batches in HBEC of COPD donor Br299. EMT assay was performed in human bronchial epithelial cells (HBEC) obtained from COPD donors at a seeding density of 2500 cells/well. Adenoviral transduction was performed one day after cell seeding. The selected combination trigger (0.5 ng/mL TGFβ1+5 ng/mL TNFα+0.5×10⁷ UV-killed NTHi bacteria/mL), which induces EMT, was added five days after transduction. Supernatant was collected three days after triggering of the cells. Fibronectin (FN) and matrix metalloproteinase-10 (MMP10) concentrations were measured using the MSD platform. FN was considered the main read-out.

An MOI of 4 was used to transduce these cells with the adenoviral library. Each screen batch included an extra plate, which contained the control panel and untransduced conditions. After completion of each batch FN and MMP10 were measured in the extra plate, the results served as a quality check for the whole batch. After completion of the data analysis for all six batches, it was decided to repeat 35 plates that did not meet acceptance criteria described in 1.6.

### 1.8 Dada Analysis

To determine which statistical method should be used for data analysis, a frequency distribution plot of all data points was generated. The frequency distribution plot shows a skewed, non-Gaussian distribution. An inter quartile range (IQR)-based normalization method is therefore most applicable, because this method is less sensitive to outliers. The IQR method uses the median (Q2) and inter quartile range (Q3-Q1) as a measure for data dispersion. When analyzing a highly skewed data set, it is possible to take an alternative measurement of data spread, for instance median and (Q1-Q2) or median and (Q3-Q2) depending on whether inhibitors or activators are of interest respectively. The choice of cut-off determines the error rate (probability of identifying a non-hit as a hit).

### 1.9 Results

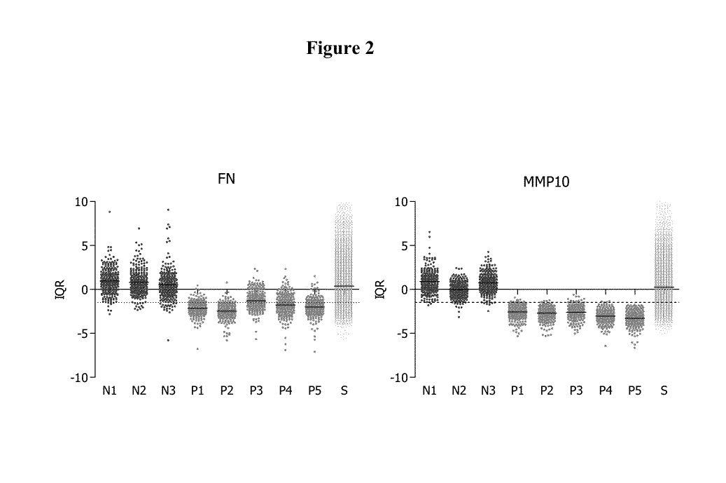

In FIG. 2 dot plots are shown of the biological duplicates of all source plates for fibronectin and MMP10 read-outs assessed in the primary screen. A separation between the negative and positive controls was observed for both read-outs. In Table 4, an overview of assay parameters for the primary screen is shown. The average Spearman correlation was above 0.4 and the average kappa value was above 0.2 for both read-outs. The average hit rate at an IQR cut-off of −1.5 was 5.5% and 8.5% for FN and MMP10, respectively.

TABLE 4
Hit rate and correlation parameters for FN and MMP10
in primary screen at an IQR cut-off of −1.5

| | # | | | | | | |
|---|---|---|---|---|---|---|---|
| | source | | | Correlation | | | |
| Read-out | plates | Hit rate (%) | | Spearman | | Kappa value | |
| FN | 143 | 5.5 | 0.0-11.4 | 0.61 | 0.34-0.88 | 0.33 | 0.0-0.87 |
| MMP10 | 143 | 8.5 | 1.2-16.7 | 0.78 | 0.56-0.92 | 0.50 | 0.10-0.91 |

In conclusion, the primary screen demonstrated a clear separation between positive and negative control viruses and correlation parameters (using Spearman correlation values and Kappa statistic values). Constructs including all double FN hits with the scores below IQR cut-off of −1.5 (n=695), with the addition of constructs that are double hits for FN below an IQR cut-off of −1.3 and double hits for MMP10 below an IQR cut-off of −1.5 (n=142) were selected. From those two sets of hits there was an overlap of 104 hits, therefore, 591 double unique FN hits were identified. From this primary screen 733 viruses (5.9% of the total number of viruses screened) were taken forward for re-screen.

TABLE 5
Overview of hit calling options. The cut-off used for hit calling is
IQR <−1.5 or −1.3 for FN and IQR <−1.5 for MMP 10. Double
indicates that both biological duplicates are below the IQR cut-off

| | | MMP10 | # Hits | Hit % |
|---|---|---|---|---|
| | FN at IQR <−1.5 | | | |
| 1 | Double | — | 695 | 5.57 |
| | FN at IQR <−1.5 | | | |
| 2 | Double | Double | 142 | 1.14 |

### Example 2

### Re-Screen Using EMT Assay

### 2.1 Background

In the re-screen the hits from the primary screen were screened again using newly repropagated viruses on a different COPD HBEC donor, Br291.

### 2.2 Positive and Negative Controls and Plate Layout

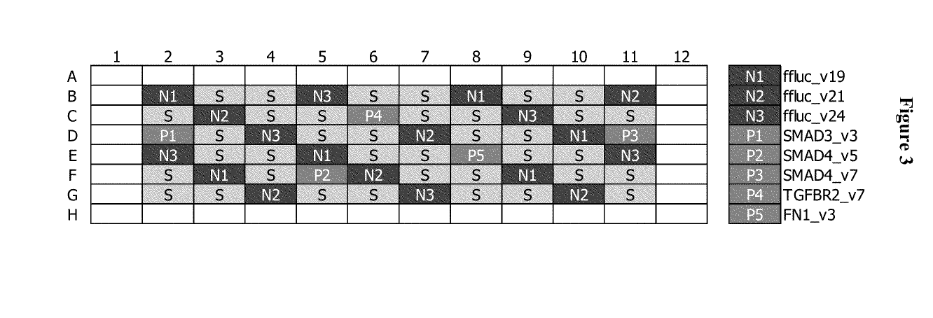

The assay setup was kept similar to the primary screen, but with a different plate layout. To enable hit calling based on the distribution of the negative controls, the plate layout included at least 30% negative controls. Five positive controls were taken along for re-screen (see Table 6). The plate layout used in re-screen is presented on FIG. 3. Positive control TGFβR1_v1 was replaced with FN1_v3. This shRNA control was used as a positive control in the FN read-out, but not for the MMP10 read-out.

TABLE 6
an overview of controls used in EMT re-screen

| Control shRNA | Sequence | SEQ ID NO: |
|---|---|---|
| Ffluc_v19 | GAATCGATATTGTTACAAC | 35 |
| Ffluc_v21 | ATATCGAGGTGAACATCAC | 36 |
| Ffluc_v24 | GCAGTCAAGTTTCCACAAC | 37 |
| SMAD3_v3 | GCTCCATCTCCTACTACGA | 38 |
| SMAD4_v5 | GTGTTCCATTGCTTACTTT | 39 |
| SMAD4_v7 | GCAGAGTAATGCTCCATCA | 40 |
| TGFBR2_v7 | GCAGTCAAGTTTCCACAAC | 42 |

### 2.3 Re-Screen Protocol

The 733 hit viruses from the primary screen were tested in one batch consisting of 14×96-well plates. Re-screen was performed in biological duplicate using the same protocol as for the primary screen (Example 1).

### 2.4 Data Analysis

For the data analysis, FN and MMP10 raw data were log transformed and subsequently normalized using the robust Z-score based on negative controls. The robust Z-score is calculated by dividing the read-out value minus the median of the negative controls, by the MAD (median absolute deviation) of the negative controls. A robust Z-score cut-off of −2 was chosen as 93% and 96% of the positive controls are below this cut-off in duplicate for FN and MMP10 respectively and most negative controls are above this cut-off in duplicate. FN was the main read-out and therefore, it was decided to only use the FN results for hit calling.

### 2.5 Results

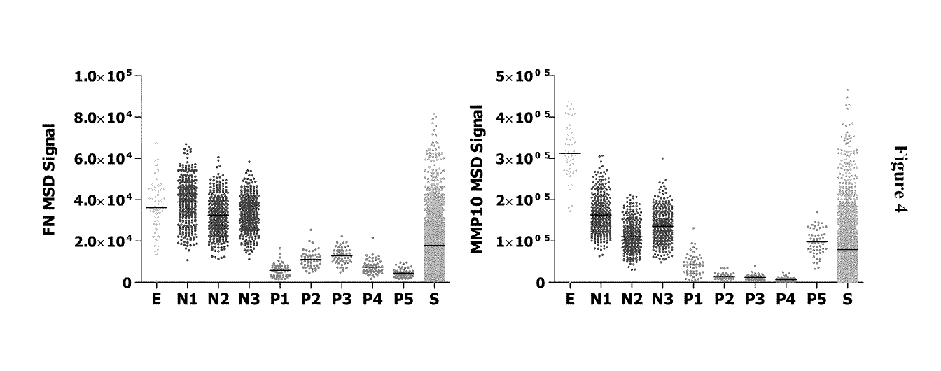

On FIG. 4 the control performance in the rescreen is shown. A clear separation between negative and positive controls was observed for both read-outs. Values for negative controls in the MMP10 read-out were lower than untransduced samples, but this was not observed in the FN read-out. A high correlation was observed between biological replicates with average Spearman rank correlation coefficient values of 0.84 (0.68-0.89) for FN and 0.92 (0.58-0.98) for MMP10.

In Table 7 an overview is provided of control performance and hit rate in rescreen using a robust Z-score cut-off of −2. Using this cut-off, 447 FN double hits were selected. Upon sequencing, 12 of these hits were excluded. Thus, in total 438 confirmed candidate Targets were taken forward into target validation.

TABLE 7
Overview of control performance and hit rate for FN and MMP10
read-out in the EMT1 rescreen using a robust Z-score cut-off of −2

| | FN | MMP10 |
|---|---|---|
| | Z-score cut-off: −2 | Z-score cut-off: −2 |
| % positive ctrl as hit | 93% | 96%* |
| % negative ctrl as hit | 0.8% | 0.8% |
| # duplicate hits | 447 | 350 |
| % of rescreened hits | 61.0% | 47.7% |

*Positive control FN1_v3 was excluded from the calculation

### Example 3

### EMT2 Validation Assay

### 3.1 Background

The EMT assay with cellular markers as read-out (designated EMT2) was employed as a secondary assay to validate the 438 confirmed candidate Targets of the re-screen. The purpose of the secondary assay was to validate targets identified in the re-screen in an EMT assay using a different read-out, the ratio of cellular expression of E-cadherin and fibronectin, measured by high content imaging on an InCell 2000 instrument (GE Healthcare) following immune staining with anti-FN and anti-E-cadherin antibodies.

### 3.2 Cells and Donors

Donor Br282 was used for the validation screen. Cells were obtained according to the protocol described in Example 1.

### 3.3 Controls and Plate Layout

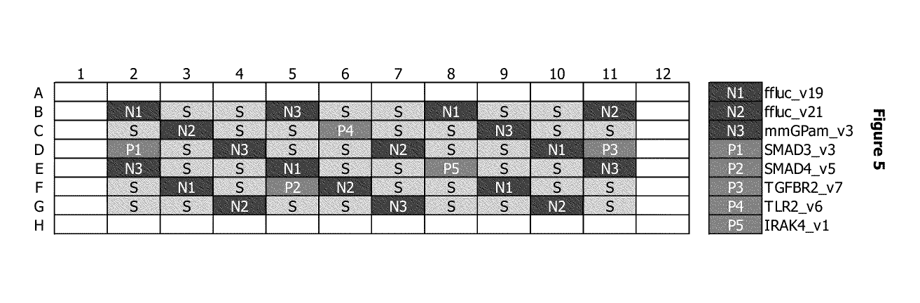

The lay-out, based on the rescreen plate lay-out, uses the 60 inner wells of a 96-well plate to reduce the plate or edge effect (FIG. 5). Furthermore, 30% of the plate was used for negative controls to facilitate hit calling. The improved distribution of the controls allowed for a better analysis of plate effects. Well G02 contained no sample but was mock transduced for nine source plates.

### 3.4 Read-Out

A different read-out was used for EMT validation assay. The ratio of E-cadherin and fibronectin (Ecad/FN) was selected as a read-out indicative for EMT.

### 3.5 Protocol

The validation assay was performed similar to the primary screen assay (Example 1), but with the exception that the cells were fixed with 4% formaldyde in PBS 72 h after adding trigger and subsequently cellular expression of FN and E-cadherin was measured using High content imaging on the InCell 2000 instrument.

### 3.6 Data Analysis

Control performance was further evaluated by analysis of the data distribution. The controls and samples of the validation screen showed a log normal distribution and were therefore log transformed before analysis. Next the robust Z-score was calculated by dividing the read-out value minus the median of the negative controls by the MAD (median absolute deviation) of the negative controls.

### 3.7 Results

An average Spearman rank correlation coefficient of 0.7 (range 0.57-0.81) was observed for all source plates that exceeded the preset cut-off of 0.4. An overview of control hit rate and sample hit rate of the validation screen, with a robust Z-score cut-off of −1.1, is provided in Table xx. Some additional targets that were strong single hits but where the biological replicate hit did not pass the cut-off were included for hit selection. A threshold was set for each replicate set to include the strong hits with a replicate that was below the average of the negative controls (replicate 1:−1.92 and <0, Replicate 2: <0 and −1.32). Using these cut-offs 96 hits were selected. Sequence analysis of the hits revealed one virus with a read-through in the sequence and one virus where the target RNA did not code for a protein. After correction this resulted in 94 targets which were taken forward into the on target analysis and were designated validated candidate Targets.

TABLE 8
Overview of control performance and hit rate
for the EMT2 validation screen

| | Z-score cut-off: | | |
|---|---|---|---|
| parameter | −1.1 | −1.92 and <0 | <0 and −1.32 |
| % pos ctrl as hit* | 96% | | |
| % neg ctrl as hit | 4.6% | | |
| # hits | 69 | 17 | 10 |
| Total hits | 96 | | |
| Hit percentage | 22% | | |

### Example 4

### On-Target Assay and Toxicity Assessment

### 4.1 Background

94 confirmed candidate targets, identified in the EMT2 validation assay, were selected for evaluation in the on target screen. Multiple adenoviral-shRNA constructs (on average 5) against the same target were produced using techniques and methods known to a skilled person. A candidate confirmed target is considered on target when at least two independent shRNA constructs (including original shRNA construct) are identified as a hit in an on target assay that is similar to the primary screen. Therefore the newly propagated constructs were tested in the EMT1 assay.

Besides testing for on target activity, cell viability was assessed in the same assay by performing the CellTiter-Blue® (CTB) cell viability assay from Promega. Cell viability was tested to eliminate false positives due to toxic effects. The confirmed candidate targets should have less than 30% cellular toxicity compared to untransduced cells. The CellTiter-Blue® assay is based on the ability of living cells to convert the redox dye resazurin into a fluorescent product resorufin at 590 nm. The more viable cells present, the higher the measured fluorescence signal will be.

### 4.2 Cells and Donors

HBEC from COPD donor Br299 were used in the on target screen and obtained using the same protocol as in Example 1.

### 4.3 Positive and Negative Controls

TABLE 9
Overview of positive and negative controls
used in “on target” assay.

| Control shRNA | sequence | SEQ ID NO: |
|---|---|---|
| Ffluc_v19 | GAATCGATATTGTTACAAC | 35 |
| Ffluc_v21 | ATATCGAGGTGAACATCAC | 36 |
| mmGPam_v3 | CTGTGTCACAATCACCCAC | 43 |
| SMAD3_v3 | GCTCCATCTCCTACTACGA | 38 |
| SMAD4_v5 | GTGTTCCATTGCTTACTTT | 39 |
| SMAD4_v7 | GCAGAGTAATGCTCCATCA | 40 |
| TLR2_v6 | GAACTGCGAGATACTGATT | 44 |
| IRAK4_v1 | ACAGATGCCTTTCTGTGAC | 45 |

### 4.4 Screening Protocol for “on Target” Screen

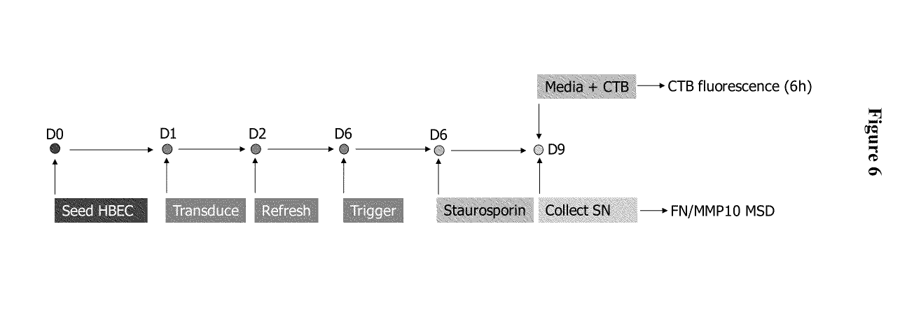

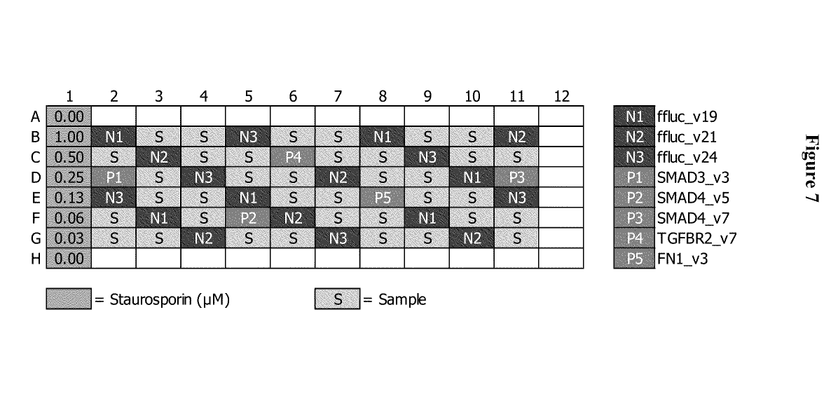

The assay setup as depicted in FIG. 6. A set of 616 shRNA viruses, targeting 94 genes (18 source plates), including the 96 original hits, were tested in the on target screen. A similar plate layout as in the rescreen was used (FIG. 7). The plate format included at least 30% negative controls to enable hit calling based on the distribution of the negative controls. To be able to determine potential cytotoxic effects of shRNAs included in the on target screen, a staurosporin standard curve was taken along on each source plate. A 30% reduction in cell viability measured by CTB fluorescence, was considered a cytotoxic effect.

### 4.5 CTB Protocol

The staurosporin concentration curve was added (two-fold dilution ranging from 1 to 0.03 μM) to all 36 cell plates as a control for decreased cell viability. At a concentration of 0.04 μM staurosporin a 30% decrease in cell viability compared to trigger only cells was observed. This concentration is in between the first and the second lowest concentration of the standard curve. Media with CTB was added to the cells after supernatant harvest and the cells were incubated for six hours at 37° C. and 5% CO₂, followed by fluorescence read-out on the EnVision® Multilabel Reader (Perkin Elmer).

### 4.6 Data Analysis

The same analysis was applied for “on target” data as in EMT rescreen (Example 2), data was log transformed, followed by a robust Z-score normalization based on the negative controls. A robust Z-score cut-off of −1.25 was chosen for both read-outs. At this cut-off all positive controls were identified as hit, while <4% of negative controls were picked up as false positive hits.

Similar data normalization was performed for the CTB data. It was log transformed, followed by the robust Z-score based on negatives normalization. The average Z-score of the lowest concentration of the standard curve (0.03 μM) was within the same range as the control panel and the trigger only samples. With the next concentration (0.06 μM) the Z-score decreased clearly, this corresponded to 0.04 μM staurosporin causing a decrease of 30% in cell viability compared to trigger only in the standard curve. Therefore it was decided to set the robust Z-score cut-off at −10, in between the two lowest staurosporin concentrations of the concentration curve.

### 4.7 Results

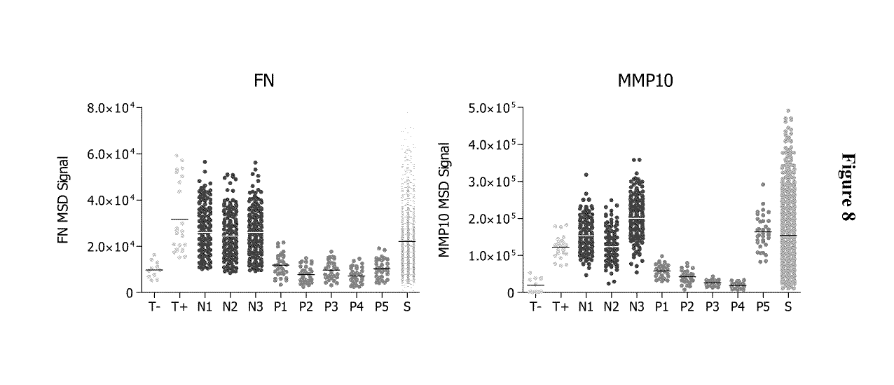

FIG. 8 shows raw data obtained from FN and MMP10 measurements of negative and positive control viruses, as well as the 616 sample viruses. A clear separation between negative and positive controls was observed for both FN and MMP10. Positive control 5 (FN1_v3) did not affect MMP10. A high correlation was observed between biological replicates with average Spearman rank correlation coefficient values of 0.78 (0.68-0.93) for FN and 0.82 (0.68-0.92) for MMP10.

In Table 10 an overview is provided of control performance and hit rate of the on target screen using a robust Z-score cut-off of −1.25. Of the 96 original hits 93 were identified as a double FN hit in the on target screen, indicating 97% hit confirmation. Using this cut-off in total 254 double FN hits and 139 double MMP10 hits were identified. The overlap between these double hits is 74 hits, which is 29% of the total FN double hits. Before assessing on target effects, CTB data were analyzed to enable exclusion of false positives due to cellular toxicity.

TABLE 10
Overview of control performance and hit rate for FN and MMP10 read-out
in the EMT on target screen using a robust Z-score cut-off of −1.25

| | FN | MMP10 |
|---|---|---|
| Parameter | Z-score cut-off: −1.25 | Z-score cut-off: −1.25 |
| % Positive ctrl as hit | 100 | 100 |
| % Negative ctrl as hit | 2.2 | 3.1 |
| # Double hits | 254 | 139 |
| % of tested viruses | 41.2 | 22.6 |
| (n = 616) | | |
| # Original hits (n = 96) | 93 | 35 |
| a double hit | | |

*Positive control FN1_v3 was excluded from the calculation

Using a robust Z-score cut-off of −10 for CTB data led to 42 double toxic viruses and 29 single toxic viruses, which resulted in total 71 toxic viruses. This group of toxic viruses consisted of 46 double FN hits and 26 double MMP10 hits; of which 19 were both FN and MMP10 double hits. Thirteen original hits of the 96 original hits were part of these 71 toxic hits and were therefore were discarded as false positive results.

### 4.8 Summary of the Results

The on target screen included both the EMT1 and the CTB assay. For both assays robust Z-score cut-offs were chosen and this led to the selection of FN and MMP10 double hits that were not toxic in the CTB assay. In Table 11 an overview is provided of the number of hits selected leading to the identification of “confirmed candidate targets” that were found to be on target. Of the 80 original hits that were a FN double hit and not toxic in the on target screen, 62 had additional knockdown constructs that targeted the same target and were a double FN hit as well. Therefore these 62 targets were designated “on target”. Similar selection was done for MMP10 and this led to 29 on targets for MMP10. Seven targets were found on target in both FN and MMP10. The 62 FN on targets were taken forward into target expression analysis and prioritization.

TABLE 11
Number of on target hits in the on target screen, taking cell toxicity into
account (CTB robust Z-score cut-off: −10), using robust Z-score cut-off
of −1.25 for both the FN and MMP10 read-out

| Parameter | FN | MMP10 |
|---|---|---|
| # Double hits | 208 | 113 |
| % of tested viruses (n = 616-71 toxic viruses = 545) | 38.2 | 20.7 |
| # Original hits (n = 96-13 toxic viruses = 83) double hit | 80 | 29 |
| # On targets including original hit | 62 | 16 |
| # ≧2 shRNA's against same target without original | 6 | 29 |

| | |
|---|---|
| # On targets including original hit FN & MMP10 | 7 |

### Example 5

### ATPlite Secondary Toxicity Assay

### 5.1 Background

In addition, a second toxicity assay was developed using the ATPlite (Perkin Elmer) assay to evaluate possible toxicity caused by target viruses. With this assay ATP, which is produced by metabolically active cells, reacts with luciferase and D-luciferin to emit light. This assay is based on the luciferase-mediated and ATP-dependent conversion of D-luciferin into oxyluciferin resulting in emission of light. The emitted light, measured as luminescence, is proportional to the ATP concentration in the sample and thus to the number of viable cells.

From the 63 targets identified in example 4, 21 targets that were of highest interest were chosen for further assessment in the ATPlite assay. For each of the 21 targets, two constructs were chosen, including the original construct.

### 5.2 Protocol

A staurosporin concentration curve (two-fold dilution ranging from 1 to 0.03 μM) was added to each cell plate as a reference for toxicity and the ATPlite read-out was performed. The highest concentration of staurosporin used decreased the luminescence to near background signal, indicating intense cellular toxicity in these wells. A concentration of 0.06 μM staurosporin resulted in a 30% decrease in cell viability compared to trigger only.

### 5.3 Results

An average Spearman rank correlation of 0.55 (0.52-0.57) was observed between biological replicates in the ATPlite assay. 0.6 μM staurosporin treatment has been shown to correspond with 30% toxicity. The data after log transformation and robust Z-score normalization based on negatives was used for the analysis of the results. The average Z-score at 0.6 μM staurosporin is −5.5 and shRNAs having a duplo Z-score below −5.5 were considered toxic. Of the 24 viruses tested targeting 12 genes, none were found to be toxic in duplo.

In conclusion, the 21 targets tested here do not show toxicity in the secondary toxicity assay in duplicate and, based on the high correlation between data from the ATPlite assay and the CTB assay described in Example 4.

### Example 6

### Whole Transcriptome Sequencing

### 6.1 Background

To confirm mRNA expression of the identified targets, mRNA from Br291 cells was isolated to perform whole transcriptome sequencing. To be relevant for fibrotic conditions, the TARGETS should be expressed in relevant tissue of the disease. To confirm the in vivo expression of the targets, HBEC and small airways epithelial cells (SAEC) were isolated from an IPF patient tissue sample obtained from Tissue Solutions. Isolation of HBEC and SAEC from the IPF tissue was performed similarly to the COPD donors (as previously described in van Wetering, 2000).

Whole transcriptome sequencing, or mRNA-seq, is a cDNA sequencing application. mRNA-seq can be used to profile the entire mRNA population and enables mapping and quantification of all transcripts. With no probes or primer design needed, mRNA-seq has the potential to provide relatively unbiased sequence information from polyA-tailed RNA for analysis of gene expression, novel transcripts, novel isoforms, alternative splice sites, and rare transcripts in a single experiment, depending on read depth.

Clustering and DNA sequencing was performed on the Illumina HiSeq 2000 (Solexa). Sequencing templates are immobilized on a flow cell surface. The Illumina flow cell is a planar optically transparent surface similar to a microscope slide, which contains a lawn of oligonucleotide anchors bound to its surface. Template DNA is prepared by ligation of adapters complimentary to the oligonucleotide anchors to the ends of target DNA. Adapted single-stranded DNAs are bound to the flow cell and amplified by solid-phase “bridge” PCR. In each PCR cycle, priming occurs by arching of the template molecule such that the adapter at its untethered end hybridizes to and is primed by a free oligonucleotide in the near vicinity on the flow cell surface. This process results in a raindrop pattern of clonally amplified templates. Sequencing proceeds by synthesis using reversible bases labeled with a fluorophore. Labeled terminators, primer, and polymerase are applied to the flow cell. After base extension and recording of the fluorescent signal at each cluster, the sequencing reagents are washed away, labels are cleaved, and the 3′ end of the incorporated base is unblocked in preparation for the next nucleotide addition. Each flow cell contains 96-120 million reads (clusters), each containing ˜1,000 copies of the same template.

6.2 Sample Preparation for the Expression Study in HBEC from COPD Donor Br291, HBEC from IPF Patient, and SAEC from IPF Patient

For the isolation of RNA of untriggered and selected combination triggered cells, HBEC of COPD donors Br291 and Br299, HBEC of an IPF patient, and SAEC of IPF patient were cultured and seeded in 96-well plates in the same manner as the rescreen (see Example 2). RNA from untriggered cells was harvested on day 1, the day that transduction would be performed. Cells were triggered on day 6 and RNA from triggered cells was harvested on day 9.

Total RNA was isolated from cultured cells using a commercially available RNA isolation kit (RNeasy Mini Kit, Qiagen). Concentration and purity was checked using the NanoDrop 2000 (Thermo Scientific), before sending the mRNA for RNA-sequencing.

The quality and integrity of the RNA sample(s) was analyzed on a RNA 6000 Lab-on-a-Chip using the Bioanalyzer 2100 (Agilent Technologies). Sample quality met the requirements for sample preparation. The Illumina® mRNA-Seq Sample Prep Kit was used to process the samples. The sample preparation was performed according the Illumina protocol “Preparing Samples for Sequencing of mRNA” (1004898 Rev. D). Briefly, mRNA was isolated from total RNA using the poly-T-oligo-attached magnetic beads. After fragmentation of the mRNA, a cDNA synthesis was performed. This was used for ligation with the sequencing adapters and PCR amplification of the resulting product. The quality and yield after sample preparation was measured with a DNA 1000 Lab-on-a-Chip (Agilent Technologies) and all samples passed the quality control. The size of the resulting products was consistent with the expected product with a broad size distribution between 300-600 bp. Br291 RNA was used for whole transcriptome sequencing and Br299 and IPF HEBEC and SAEC RNA was used for real time PCR.

Clustering and DNA sequencing using the Illumina HiSeq 2000 (Illumina) were performed according manufacturer's protocols. A total of 6.5 pmol of DNA was used. Two sequencing reads of 100 cycles each using the Read 1 sequencing and Read 2 sequencing primers were performed with the flow cell. From 39 of 63 TARGETS identified in the on target screen, cDNA was quantified on the LightCycler® 480 Real-Time PCR System (Roche Diagnostics) using TaqMan® Fast Advanced Master Mix (Life Technologies, cat. 4444964) with commercially available validated TaqMan® Assays (Life Technologies or Qiagen). A set of four housekeeping genes was tested to confirm the quality of the sample.

### 6.3 Primary Data Analysis and Results

Image analysis, base-calling, and quality check was performed with the Illumina data analysis pipeline RTA v1.13.48 and/or OLB v1.9 and CASAVA v1.8.2.

QA analysis performed to evaluate the quality of an Illumina sequencing run was based on quality metrics for a standard run of good quality using the Solexa technology. All lanes of the flow cell passed the QA analysis. Additionally, detailed error rate information based on an Illumina supplied Phi X control was reported. The Phi X control is spiked into the sample in a small amount (up to 5% of the reads). The reads from the Illumina control DNA are removed by the Illumina pipeline during processing of the data. The error rate is calculated after alignment of the reads passing the quality filter to the Phi X reference genome using the ELAND aligner in the Illumina pipeline. All error rates were within the allowed criteria.

6.4 Data analysis

Reads obtained from the Illumina HiSeq 2000 sequencer were filtered by quality scores with a minimum threshold of Q25 and minimum length of 50 bases.

Reads were then aligned to the human reference genome (hg19) with the Bowtie v0.12.7 aligner for each sample. New isoforms were identified with the Cufflinks v2.02 package using default settings and the known transcriptome annotation as mask (Homo_{—}sapiens.GRCh37.65.gff). After new isoform discovery for each sample, the newly detected isoforms were merged for all samples and added to the standard transcriptome annotation. Finally, FPKM (Fragments Per Kilobase of transcript per Million fragments mapped) values were calculated with Cufflinks for each sample and reported in the default Cufflinks output. The FPKM values are a quantitative representation of the mRNAs in the samples and therefore in the cells used for the mRNA-seq analysis and the screening assays. Highly abundant mRNAs result in high FPKM values whereas low FPKM values represent low copy numbers of the mRNA.

### 6.5 Results

The results for the identified 12 TARGETs are included in Table 17. Out of 63 targets originally identified in the on target screen were subjected to whole transcriptome sequencing. Of these 63 TARGETS, the selected 12 TARGETs showed FPKM values >0.00 under triggered (+T) or untriggered (−T) conditions, confirming that those targets are expressed in HBEC. Results from the real time PCR studies indicate that all 12 TARGETs showed Ct values of 40 or lower in Br299 cells and/or IPF HBEC and SAEC, confirming that those targets are expressed in those cells.

### Example 7

### Testing siRNAs Against the TARGETs in EMT Assay

### 7.1 Background

To exclude that the shRNA knockdown constructs have an effect on expression of a different mRNA then the intended mRNA, so called off-target effect, an on-target validation was performed with the confirmed candidate Targets using siRNA constructs against selected TARGETS.

### 7.2 Positive and Negative Controls

siRNA against SMAD3 and SMAD4 were used as positive controls and non-targeting siRNA (Thermo Fisher Scientific Biosciences GMBH) was used as a negative control.

### 7.3 Cell Cultures

HBEC were obtained from the Dept of Pulmonology (LUMC, Leiden, The Netherlands). HBEC were derived from lung resection tissue of patients undergoing surgery for lung tumors. Bronchial epithelial cells were isolated by protease digestion and cultured as previously described (van Wetering, 2000).

7.4 Assay Protocol for siRNA Screen

The experimental setup was as follows: On day zero 2500 cells/well of HBEC were seeded in 96-well plates coated with 32 μg/mL PureCol coating (Advanced Biomatrix Cat#5005-B). Three days later the siRNA transfection was preformed. Cells were transfected using 0.02 μL/well of Dharmafect 1 (Thermo, Cat # T-2001-03). OnTarget Plus siRNA (Thermo Fisher Scientific Biosciences GMBH) in the final concentration of 20 nM were used as smart pools of 4 constructs per well. One day after the combination trigger inducing EMT (0.5 ng/mL TGFβ1+5 ng/mL TNFα+0.5×10⁷ UV-killed NTHi bacteria/mL) was added. On day 6 Staurosporin was added to the cells in control wells (one row on each plate). On day 7 the supernatant was harvested. On the same day RNA isolation was performed using standard MagMax Total RNA isolation kit (Ambion, Cat # AM1830). Cell Titer Blue assay (Promega, Cat # G808B) was performed on the same day. FN was measured using the Mesoscal Discovery (MSD) platform on a SECTOR® Imager 6000 instrument (MSD) using in-house developed assay as described in Example 1

### 7.5 Data Analysis

Normalized percentage inhibition (NPI) analysis was used to quantify the effect of siRNA constructs on the read-out. SMAD3 or SMAD4 siRNA was used as a positive control and non-targeting siRNA as a negative control in the calculations. Normalized percentage inhibition (NPI) was calculated by dividing the difference between sample measurements and the average of positive controls through the difference between positive and negative controls.

### Example 8

### TARGET Expression in Animal Models of Fibrosis

### 8.1 Background

To study the expression of the TAREGT genes in vivo, several mouse and rat models of fibrosis were tested and expression in specific tissues like kidney, lung and skin were determined

### 8.2 Mouse UUO (Unilateral Ureteral Obstruction) Renal Fibrosis Model

Unilateral ureteral obstruction was performed on Balb/c female mice (from Harlan-France), with 10 mice/group. On day 0, mice were anaesthetized by intra-peritoneal injection and after incision of the skin, the left ureter was dissected out and ligatured with 4.0 silk at two points along its length. The ureter was then sectioned between the 2 ligatures. Intact mice were used as control. Mice were sacrificed by exsanguinations with scissors under anaesthesia after 10 or 21 days.

### 8.3 Rat 5/6 NTX (5/6 Nephrectomy) Renal Fibrosis Model

Nephrectomy was performed on Sprague-Dawley male rats (from CERJ-France), with 10 rats/group. At Day 0, rats were anaesthetized and after incision of the skin, the kidney capsule was removed while preserving the adrenal gland. The renal hilum was ligated and right kidney was removed. The ends of the left kidney are cut with a scalpel resulting in 5/6 nephrectomy. Rats were sacrificed after 4 or 8 weeks.

### 8.4 Mouse BLM (Bleomycine) Pulmonary Fibrosis Model

Lung fibrosis was induced on CD1 male mice (from CERJ-France) for bleomycin i.v. administration with 6 to 8 mice/group and on C57/B16 J female mice (from Janvier) for bleomycin i.t. administration with 14 mice/group.

For intravenous administration mice were injected intravenously (i.v.) with bleomycin (10 mg/kg; 100 μl/mouse) or saline as a control once per day for the first five consecutive days (Oku et al., 2004). Mice were sacrificed by exsanguinations with scissors under anaesthesia after 3 or 6 weeks.

For intra-peritoneal administration mice were anaesthetized by intra-peritoneal injection (under a volume of 10 mL/kg) of anaesthetic solution (18 mL NaCl 0.9%+0.5 mL xylazine (5 mg/kg)+1.5 mL ketamine (75 mg/kg)). Bleomycin solution at 2 U/kg or saline was administered by intratracheal route (10 mg/kg; 40 μL/mouse). Mice were sacrificed by exsanguinations with scissors under anaesthesia after 3 weeks.

### 8.5 Mouse Scleroderma Model

Scleroderma was induced on Balb/c female mice (from CERJ-France), with 15 mice per group. On day 0 mice were anesthetised by intra-peritoneal injection of a solution (Xylazine 5 mg/kg, ketamine 75 mg/kg) and shaved. A volume of 100 μl of bleomycin solution at 1 mg/ml or saline was injected subcutaneously with a 26 g needle into the shaved backs of mice. Bleomycin was injected 5 days per week for 3 consecutive weeks. The total experimental period was 6 weeks. Mice were sacrificed by exsanguinations with scissors under anaesthesia after 6 weeks.

### 8.6 Gene Expression and Regulation in Animal Fibrosis Models

At the end of the in vivo experiment, animals were sacrificed and tissues (½ mouse kidney for UUO model, ⅓ rat kidney for ⅚ NTX model, a piece of skin for mouse scleroderma model and 1 lobe of lung for mouse lung fibrosis model) were collected in 2 ml-microtubes (Ozyme #03961-1-405.2) containing RNALater® stabilization solution (Ambion #AM7021). Tissues were disrupted with 1.4 mm ceramic beads (Ozyme #03961-1-103, BER1042) in a Precellys® 24 Tissue Homogenizer (Bertin Technologies). Total RNA was isolated, subjected to recombinant DNase digestion and purified using Qiazol® (Qiagen #79306) and NucleoSpin® RNA kit (Macherey-Nagel #740955.250) as recommended by the manufacturers. RNA was eluted with 60 μl RNase-free water. RNA concentration and purity were determined by absorbance at 260, 280 and 230 nm. cDNA was prepared from 500 ng total RNA by reverse transcription using a high-capacity cDNA RT kit (Applied Biosystems #4368814). 5 μl of 10 times diluted cDNA preparations were used for real-time quantitative PCR. qPCR was performed with gene-specific primers from Qiagen using SYBR Green technology. Reactions were carried out with a denaturation step at 95° C. for 5 min followed by 40 cycles (95° C. for 10 sec, 60° C. for 30 sec) in a ViiA7 real-time PCR system (Applied Biosystems).

The following rodent β-actin primers (Eurogentec) were used: 5′-ACCCTGTGCTGCTCACCG-3′ (forward primer SEQ ID NO: 77) and 5′-AGGTCTCAAACATGATCTGGGTC-3′ (reverse primer SEQ ID NO: 78).

Mouse and rat assay mixes are listed in the table below (table 12).

TABLE 12
Mouse and rat assay mixes (Qiagen)

| Target | mouse | rat |
|---|---|---|
| CLK2 | QT02326380 | QT01613129 |
| CSNK2A2 | QT00124082 | QT01579935 |
| IGFBP7 | QT02419662 | QT01590001 |
| OTUD6B | QT02273110 | QT01583981 |
| PARP1 | QT00157584 | QT00182609 |
| STK4 | QT00151515 | QT01587460 |
| F2R | QT00119812 | |
| EFEMP2 | QT00162134 | |

### 8.7 Data Analysis

Expression levels of each gene were estimated by their threshold cycle (C_{T}) values in control animals.

The quantification of relative changes in gene expression were expressed using the 2^{-ΔΔC}_{T} method (where ΔΔC_{T}=(C_{T-}target−C_{T}β-actin)_{diseased animal}−(C_{T-}target−C_{T}β-actin)_{control animal}. Statistical analysis on 2^{-ΔΔC}_{T} values were performed using unpaired Student's t-test versus control group (***: p<0.001; **: p<0.01; *: p<0.05)

### 8.8 Results

All tested mRNA are well expressed in fibrotic tissues (kidney, lung and skin) (see Table 13)

TABLE 13
mRNA expression levels in intact animals

| | STK4 | CLK2 | CSNK2A2 | IGFBP7 | OTUD6B | PARP1 | EFEMP2 | F2R |
|---|---|---|---|---|---|---|---|---|
| Mouse UUO | 22.9 | 22.2 | 21.4 | 24.1 | 21.9 | 21 | 24.7 | 23.8 |
| (10 days) | | | | | | | | |
| Mouse UUO | 22.8 | 22.3 | 21.5 | 24.4 | 22.1 | 21.4 | 24.1 | 23.2 |
| (21 days) | | | | | | | | |
| Rat NTX | 21.4 | 20.4 | 21 | 14.5 | 21.1 | 20.5 | | |
| (4 week) | | | | | | | | |
| Rat NTX | 21.5 | 20.7 | 21.7 | 15.4 | 21 | 21.5 | | |
| (8 week) | | | | | | | | |
| Mouse BLM | 21.2 | 21.3 | 22.2 | 26.7 | 22.8 | 22.1 | | |
| (i.v. 3 w) | | | | | | | | |
| Mouse BLM | 20 | 20.5 | 22.7 | 25.8 | 22.6 | 21.4 | | |
| (i.v. 6 weeks) | | | | | | | | |
| Mouse BLM | | | 23 | | 23.9 | 24 | 23.4 | 21 |
| (single i.t.) | | | | | | | | |
| Mouse SCL | 24.5 | 22.2 | 21.4 | 27.4 | 23.4 | 23.6 | 25.2 | 24.8 |

(Ct > 30: low, 25 < Ct < 30: medium, Ct < 25: high)

Many genes are up or down regulated in mouse UUO model whereas only few regulations were observed in rat NTX model (4 & 8 weeks), and in lung and skin fibrosis models. EFEMP2 and F2R genes are up regulated in at least one mouse fibrosis model. (see Table 14)

TABLE 14
qPCR analysis of the fibrosis models

| | STK4 | PARP1 | CLK2 | CSNK2A2 | IGFBP7 | OTUD6B | EFEMP2 | F2R |
|---|---|---|---|---|---|---|---|---|
| Mouse | 1.6 (***) | ns | ns | ns | −1.8 *** | −2.1 *** | 2.1 *** | 2.5 *** |
| UUO | | | | | | | | |
| (10 days) | | | | | | | | |
| Mouse | 1.8 *** | −2.8 *** | ns | −2.5 *** | −2.5 *** | −3.7 *** | 1.7 (***) | 2.4 *** |
| UUO | | | | | | | | |
| (21 days) | | | | | | | | |
| Rat NTX | ns | ns | ns | ns | ns | ns | | |
| (4 week) | | | | | | | | |
| Rat NTX | −1.6 (*) | ns | −1.4 (*) | ns | −1.4 (**) | −1.5 (*) | | |
| (8 week) | | | | | | | | |
| Mouse | ns | 1.7 (***) | 1.3 (*) | 1.9 * | 3.8 *** | 1.6 (**) | | |
| BLM | | | | | | | | |
| (i.v. 3 w) | | | | | | | | |
| Mouse | ns | ns | ns | ns | 1.8 ** | ns | | |
| BLM | | | | | | | | |
| (i.v. | | | | | | | | |
| 6 weeks) | | | | | | | | |
| Mouse | | −1.3 (***) | | ns | | −1.5 (***) | 1.4 (***) | 1.4 (**) |
| BLM | | | | | | | | |
| (single i.t.) | | | | | | | | |
| Mouse SCL | 1.5 (*) | 1.3 (*) | ns | 1.6 (*) | ns | 1.5 (***) | ns | 2 ** |

(fold > 1.8: significant fold induction vs intact animals; fold < −1.8: significant fold inhibition vs intact animals; ns: no significant change; *** p < 0.001; ** p < 0.01; * p < 0.05)

TABLE 15
Overview of the performance of TARGETs in the primary screen, rescreen, and EMT2 validation assay. The first column shows the Target
gene symbol. Duplicate IQR-scores are shown for the primary EMT1 FN and MMP10 screens, where a cut-off of duplicate IQR ≦ −1.5
for FN and duplicate IQR ≦ −1.3 was used. The rescreen robust Z-scores are shown for both the FN and MMP10 read-outs. A cut-
off of duplicate robust Z ≦ −2.0 for FN was used. Results of the EMT2 validation assay are shown with duplicate Z-scores
where a cut-off of duplicate robust Z ≦ −1.1 in combination with the following criteria: replicate 1: −1.92 and <0, Replicate 2: <0 and −1.32

| | Primary screen FN1 | | Primary screen MMP10 | | Rescreen FN1 | | Rescreen MMP10 | | EMT2 assay | |
|---|---|---|---|---|---|---|---|---|---|---|
| TARGET | IQR-score 1 | IQR-score 2 | IQR-score 1 | IQR-score 2 | Z-score 1 | Z-score 2 | Z-score 1 | Z-score 2 | Z-score 1 | Z-score 2 |
| ADRBK2 | −1.93 | −2.43 | −1.61 | −2.52 | −4.02 | −3.47 | −2.02 | −2.44 | −1.46 | −1.34 |
| APOL1 | −1.94 | −2.00 | −0.17 | 1.72 | −10.21 | −3.98 | −14.04 | −6.24 | −5.30 | −1.33 |
| CLK2 | −1.97 | −3.23 | 0.76 | 1.14 | −4.92 | −4.40 | −0.19 | 0.25 | −1.54 | −2.37 |
| CSNK2A2 | −1.89 | −2.59 | 0.16 | −0.66 | −16.74 | −10.14 | −6.42 | −4.17 | −1.50 | −2.36 |
| EFEMP2 | −2.20 | −1.95 | −0.81 | −0.10 | −15.92 | −8.82 | −7.97 | −4.49 | −0.43 | −2.08 |
| F2R | −3.94 | −2.04 | −3.52 | −2.59 | −7.45 | −3.86 | −10.00 | −7.44 | −1.14 | −1.61 |
| IGFBP7 | −2.45 | −1.99 | −2.42 | −0.64 | −2.97 | −2.50 | −4.19 | −2.08 | −2.30 | −0.84 |
| OTUD6B | −2.94 | −2.45 | −1.57 | −0.95 | −12.73 | −7.01 | −8.79 | −5.79 | −3.33 | −3.29 |
| PARP1 | −2.29 | −1.90 | −0.68 | −0.22 | −2.83 | −4.56 | −2.52 | −3.58 | −2.86 | −1.86 |
| SLC15A3 | −2.73 | −2.19 | −1.45 | −1.15 | −7.08 | −4.11 | −3.60 | −2.60 | −0.80 | −1.97 |
| STK4 | −3.00 | −1.90 | −3.99 | −1.82 | −6.73 | −9.08 | −7.89 | −6.14 | −1.54 | −1.37 |
| WNT5A | −1.59 | −1.84 | −1.30 | −0.37 | −3.64 | −7.01 | 0.92 | −0.95 | −5.34 | −1.57 |

TABLE 16
Overview of the performance of the TARGETs in the on target validation.
This table gives an overview of the performance of the confirmed TARGETs in the on target assays.
The confirmed candidate TARGET gene symbol and a knock-down sequence of the adenoviral
constructs are shown. Results for the shRNAs which were considered a hit are shown and in addition
the shRNA that originally was a hit (bold), and the “Both” column shows if this shRNA is a hit again
in both OT assays (Yes/ No). Duplicate results are shown for FN and MMP10 read-outs in the EMT
on target screen. A cut-off of duplicate robust Z ≦ −1.25 was used. CTB results for toxicity
assessment is shown and a duplicate robust Z ≦ −10. Hits were included based on FN inhibition and
non-toxic effect in the CTB assay. The secondary ATPlite toxicity assay was performed and a cut-off
of duplicate robust Z was used.

| | | | | | OT MMP10 | | | | |
|---|---|---|---|---|---|---|---|---|---|
| | | | OT FN Screen | | Screen | | OT CTB assay | | |
| | | SEQ | Z- | Z- | Z- | Z- | Z- | Z- | |
| TARGET | Sequence | ID NO | score 1 | score 2 | score 1 | score 2 | score 1 | score 2 | Both |
| ADRBK2 | ACTTCTGAGAGGTCACAGC | 46 | -8.84 | -7.86 | -2.20 | -2.42 | -2.43 | -5.13 | yes |
| ADRBK2 | GAACACGTACAAAGTCATT | 47 | -4.88 | -5.03 | -11.24 | -6.24 | -7.55 | -4.92 | no |
| APOL1 | GGATGGAGTTGGGAATCAC | 48 | -3.28 | -3.44 | -2.26 | -2.43 | -1.14 | -3.01 | yes |
| APOL1 | GAGGATGCCATTAAGTATT | 49 | -1.75 | -1.84 | 0.57 | 0.87 | -0.78 | -0.88 | no |
| APOL1 | GAGGCAGCCTTGTACTCTT | 50 | -2.42 | -4.42 | 1.32 | -0.49 | -0.51 | 2.33 | no |
| CLK2 | GGATCTTGGGTCCTATCCC | 51 | -2.90 | -3.08 | -0.02 | -0.23 | -0.73 | -2.03 | no |
| CLK2 | TGAATACTATGTGGGATTC | 52 | -3.30 | -3.74 | 1.96 | 0.95 | -3.43 | -3.45 | yes |
| CLK2 | TCAGCTGGGCGCTATGTTC | 53 | -3.01 | -2.98 | -0.41 | 0.45 | -3.97 | -4.28 | no |
| CSNK2A2 | GACTGGAAAGCGACGGGTC | 54 | -3.47 | -3.15 | 0.62 | 0.94 | -4.02 | -5.83 | no |
| CSNK2A2 | AGGCTCACTTGCCTTTGGC | 55 | -4.43 | -6.07 | 0.92 | 0.18 | -4.29 | -8.29 | yes |
| EFEMP2 | TGATGGTTACCGCAAGATC | 56 | -2.74 | -3.69 | -3.35 | -5.05 | 0.64 | -2.23 | yes |
| EFEMP2 | CCAAACCTGTGTCAACTTC | 57 | -8.59 | -3.69 | -0.60 | -1.94 | -1.43 | 0.38 | no |
| F2R | GATCCCAGCAGTTATAACA | 58 | -2.47 | -6.55 | -3.06 | -3.41 | -4.03 | -2.02 | no |
| F2R | TGAAGGTCAAGAAGCCGGC | 59 | -6.20 | -8.92 | -1.71 | -1.19 | -6.59 | -4.59 | yes |
| IGFBP7 | AACCTGGCCATTCAGACCC | 60 | -2.56 | -2.96 | -1.88 | -0.06 | -7.10 | -2.00 | yes |
| IGFBP7 | CAATTCCCAAGGACAGGCT | 61 | -1.65 | -1.44 | -2.21 | -2.31 | 0.59 | 0.86 | no |
| OTUD6B | CAGATTCCATCTGATGGCC | 62 | -4.99 | -5.21 | -2.75 | -2.79 | -3.19 | -2.25 | yes |
| OTUD6B | GAATTTCAGAAGTACTGTG | 63 | -3.62 | -3.76 | 1.53 | 2.02 | -2.12 | -3.06 | no |
| PARP1 | GTCCAACAGAAGTACGTGC | 64 | -3.09 | -2.52 | -0.07 | 1.09 | 1.00 | -1.33 | no |
| PARP1 | GGCCATGATTGAGAAACTC | 65 | -4.35 | -4.03 | -5.08 | -3.44 | 1.09 | 1.89 | no |
| PARP1 | GAAGGAGCTACTCATCTTC | 66 | -2.81 | -4.82 | -1.93 | 0.70 | -2.25 | -4.39 | yes |
| PARP1 | CAAGAGCGATGCCTATTAC | 67 | -3.19 | -2.65 | 1.39 | 0.32 | -3.77 | -3.83 | no |
| SLC15A3 | CATCAGCTTCCTGCTGGGC | 68 | -4.29 | -5.66 | -1.18 | -0.35 | -2.64 | -2.21 | yes |
| SLC15A3 | GATGGAGCGCTTACACTAC | 69 | -4.37 | -5.75 | -4.95 | -3.43 | -3.34 | -1.32 | no |
| SLC15A3 | GAGTTTGCCTACTCAGAGG | 70 | -4.52 | -4.05 | -4.35 | -1.35 | 0.19 | 1.51 | no |
| SLC15A3 | CACGGCTCTCCTATTTGTC | 71 | -1.73 | -1.64 | 0.88 | 0.95 | -5.58 | -4.56 | no |
| STK4 | GAGTTGGACAGTGGAGGAC | 72 | -3.99 | -7.31 | -4.52 | -6.59 | -3.13 | -4.22 | yes |
| STK4 | GAAACCATCCTTTCTTGAA | 73 | -2.09 | -2.91 | -0.19 | 0.06 | -1.31 | -2.09 | no |
| WNT5A | AGACCTGGTCTACATCGAC | 74 | -2.80 | -4.17 | -2.88 | -2.12 | -8.40 | -6.74 | no |
| WNT5A | TCGCTAGGTATGAATAACC | 75 | -2.04 | -2.58 | 0.15 | -0.75 | -0.46 | -2.90 | yes |

TABLE 17
Overview of the expression of the TARGETs. The TARGETs are shown with the corresponding gene
class of the Target. Expression data is shown as EST per Million in lungs. Expression data
obtained from RNA-seq is shown as an FPKM value of one normal HBEC donor BR291, either non-
triggered (T−) or triggered (T+) with combination trigger as described in the example.
mRNA expression of COPD HBEC donor Br299, IPF HBEC, and IPF SAEC are shown as Ct values.

| | | Expression | | | | | | | | |
|---|---|---|---|---|---|---|---|---|---|---|
| | | EST | FPKM | FPKM | qPCR | qPCR | qPCR | qPCR | qPCR | qPCR |
| | | per | HBEC | HBEC | HBEC | HBEC | HBEC | HBEC | SAEC | SAEC |
| | | Million | Br291 | Br291 | Br299 | Br299 | IPF | IPF | IPF | IPF |
| Gene | Gene class | in lung | T− | T+ | T− | T+ | T− | T+ | T− | T+ |
| ADRBK2 | Kinase | 47.48 | 5.62 | 3.68 | 32.62 | 30.86 | 31.73 | 31.45 | 31.89 | 32.20 |
| APOL1 | Transporter | 163.22 | 6.59 | 8.94 | 35.00 | 32.73 | 35.05 | 33.96 | 33.57 | 33.78 |
| CLK2 | Kinase | 47.48 | 22.36 | 16.75 | 32.22 | 30.79 | 31.59 | 31.46 | 31.63 | 32.27 |
| CSNK2A2 | Kinase | 71.22 | 18.90 | 22.16 | 30.98 | 28.89 | 30.01 | 29.62 | 30.30 | 30.16 |
| EFEMP2 | Secreted/ | 827.96 | 10.92 | 9.97 | 34.50 | 31.97 | 32.82 | 31.96 | 32.06 | 31.58 |
| | Extracellular | | | | | | | | | |
| F2R | GPCR | 32.64 | 4.95 | 13.55 | 40.00 | 38.10 | 40.00 | 39.04 | 40.00 | 40.00 |
| IGFBP7 | Transporter | 109.80 | 146.29 | 226.70 | 27.83 | 25.06 | 27.42 | 26.27 | 26.90 | 26.18 |
| OTUD6B | Other | 17.81 | 6.87 | 6.67 | 30.77 | 29.58 | 30.08 | 30.50 | 30.22 | 31.87 |
| PARP1 | Enzyme | 142.44 | 39.35 | 20.90 | 33.52 | 32.67 | 32.34 | 33.37 | 32.54 | 33.62 |
| SLC15A3 | Transporter | 32.64 | 2.12 | 3.67 | 38.83 | 35.54 | 37.60 | 36.04 | 37.26 | 35.43 |
| STK4 | Kinase | 35.61 | 6.79 | 7.33 | 31.16 | 29.45 | 30.27 | 30.19 | 30.61 | 31.27 |
| WNT5A | Secreted/ | 38.58 | 0.72 | 1.63 | 35.62 | 32.63 | 35.67 | 33.49 | 36.09 | 34.10 |
| | Extracellular | | | | | | | | | |

### REFERENCES

Borthwick L A, Mcllroy E I, Gorowiec M R et al. Inflammation and epithelial to mesenchymal transition in lung transplant recipients: role in dysregulated epithelial wound repair. Am J Transplant 2010; 10:498-509
Borthwick L A, Sunny S S, Oliphant V et al. Pseudomonas aeruginosa accentuates epithelial-to-mesenchymal transition in the airway. Eur Respir J 2011; 37:1237-47
Camara J, Jarai G. Epithelial-mesenchymal transition in primary human bronchial epithelial cells is Smad-dependent and enhanced by fibronectin and TNF-alpha. Fibrogenesis Tissue Repair 2010; 3:2
Choi S S, Diehl A M. Epithelial-to-mesenchymal transitions in the liver. Hepatology 2009; 50:2007-13
Firrincieli D, Boissan M, Chignard N. Epithelial-mesenchymal transition in the liver. Gastroenterol Clin Biol 2010; 34:523-8
Hay E D: The mesenchymal cell, its role in the embryo, and the remarkable signaling mechanisms that create it. Dev Dyn 2005, 233:706-720
Kasai H, Allen J T, Mason R M et al. TGF-beta1 induces human alveolar epithelial to mesenchymal cell transition (EMT). Respir Res 2005; 6:56.
Lekkerkerker A N, Aarbiou J, van Es T, Janssen R A. Cellular players in lung fibrosis. Curr Pharm Des. 2012; 18: 4093-4102
Bethany B. Moore and Cory M. Hogaboam Murine models of pulmonary fibrosis. Am J Physiol Lung Cell Mol Physiol 294:L152-L160, 2008
Shimamura M, Murphy-Ullrich J E, Britt W J. Human cytomegalovirus induces TGF-beta1 activation in renal tubular epithelial cells after epithelial-to-mesenchymal transition. PLoS Pathog 2010; 6:e1001170
Peter Starkel, I. A. Leclercq Animal models for the study of hepatic fibrosis. Best Practice & Research Clinical Gastroenterology, Volume 25, Issue 2, April 2011, Pages 319-333
Thiery J P: Epithelial-mesenchymal transitions in tumour progression. Nat Rev Cancer 2002, 2:442-454
van Wetering S, van der Linden A C, van Sterkenburg M A, de Boer W I, Kuijpers A L, Schalkwijk J, Hiemstra P S. Am J Physiol Lung Cell Mol Physiol. 2000 January; 278(1):L51-8.
Wilson M S, Wynn T A: Pulmonary fibrosis: pathogenesis, etiology and regulation. Mucosal Immunol 2009, 2:103-121
Wynn T A. Fibrotic disease and the T(H)1/T(H)2 paradigm. Nat Rev Immunol. 2004; 4:583-94 Zavadil J, Bottinger E P: TGF-beta and epithelial-to-mesenchymal transitions. Oncogene 2005, 24:5764-5774
Michael Zeisberg, Mary A. Soubasakos, Raghu Kalluri Animal Models of Renal Fibrosis. Fibrosis Research. Methods in Molecular Medicine, Volume 117, 2005, pp 261-272

1. A method for identifying a compound useful for the treatment of a disease associated with epithelial mesenchymal transition, said method comprising:
a) contacting a test compound with a polypeptide comprising an amino acid sequence selected from the group consisting of SEQ ID NOs: 21-22, 18-20 and 23-34, functional fragments and derivatives thereof, or with a cell expressing said polypeptide;
b) determining a binding affinity of the test compound to said polypeptide, or measuring expression, amount or an activity of said polypeptide;
c) contacting the test compound with a population of epithelial cells;
d) measuring a property related to epithelial mesenchymal transition; and
e) identifying a compound capable of capable of inhibiting of epithelial mesenchymal transition and demonstrating binding affinity to said polypeptide or reducing or inhibiting the expression, amount or an activity of said polypeptide.

2. (canceled)

3. (canceled)

4. A method for identifying a compound inhibiting epithelial mesenchymal transition (EMT), said method comprising:
a) contacting a test compound with a polypeptide comprising an amino acid sequence selected from the group consisting of SEQ ID NOs: 21-22, 18-20 and 23-34, functional fragments and functional derivatives thereof or with a nucleic acid encoding an amino acid selected from the group consisting of SEQ ID NOs: 21-22, 18-20 and 23-34 or a functional derivative thereof;
b) measuring the expression or an activity of said polypeptide;
c) contacting the test compound with a population of epithelial cells;
d) measuring a property related to EMT; and
e) identifying a compound inhibiting EMT and inhibiting the expression or an activity of said polypeptide.

5. (canceled)

6. The method according to claim 4, wherein the nucleic acid is selected from the group consisting of SEQ ID NOs: 4-5, 1-3 and 6-17.

7. The method of claim 1, wherein said disease is a fibrotic disease.

8. The method of claim 1, wherein said disease is a cancer.

9. The method according to claim 1 or 4, which additionally comprises the step of comparing the compound to be tested to a control.

10. The method of claim 1 or 4, wherein said polypeptide is coupled to a detectable label.

11. The method according to claim 1 or 4, wherein said polypeptide sequence in steps (a) and (b) is present in an in vitro cell-free preparation.

12. The method according to claim 1 or 4, wherein said polypeptide sequence in steps (a) and (b) is present in a cell.

13. The method according to claim 1, wherein the cell naturally expresses said polypeptide.

14. The method according to claim 1, wherein the cell has been engineered so as to express said polypeptide.

15. (canceled)

16. The method of claim 1, wherein said cell is an epithelial cell.

17. (canceled)

18. The method according to claim 16, wherein said cell is a human bronchial epithelial cell.

19. The method of claim 1 or 4, wherein said property is the inhibition of release and/or expression of a marker of epithelial mesenchymal transition (EMT marker).

20. The method of claim 19 wherein said property is the expression and/or release of a marker selected from the group consisting of matrix Metalloproteases (MMPs), cellular fibronectin (FN), E-cadherin, soluble fibronectin, and vimentin.

21. (canceled)

22. The method according to claim 16 wherein said cell has been triggered by a factor which induces epithelial mesenchymal transition (EMT inducing factor).

23. The method according to claim 22, wherein said EMT inducing factor is selected from a group consisting of TGFβ, IL-1β, TNFα, and a bacterial challenge.

24. (canceled)

25. The method according to claim 1, wherein said test compound is selected from the group consisting of an antisense polynucleotide, a ribozyme, short-hairpin RNA (shRNA), microRNA (miRNA) and a small interfering RNA (siRNA).

26. The method according to claim 25, wherein said test compound comprises a nucleic acid sequence complementary to, or engineered from, a naturally-occurring polynucleotide sequence of about 17 to about 30 contiguous nucleotides of a nucleic acid sequence selected from the group consisting of SEQ ID NO: 4-5, 1-3 and 6-17.

27. (canceled)

28. (canceled)

29. The method according to claim 25, wherein said antisense polynucleotide, said siRNA or said shRNA comprise an antisense strand of 17-25 nucleotides complementary to a sense strand, wherein said sense strand is selected from 17-25 continuous nucleotides of a nucleic acid sequence selected from the group consisting of SEQ ID NO: 4-5, 1-3 and 6-17.

30. (canceled)

31. The method according to claim 1 or 4, wherein said compound is an antibody or an antibody fragment.

32. A method for treatment of a disease associated with epithelial mesenchymal transition in a mammal comprising administering to said mammal a pharmaceutical composition comprising an antibody or a fragment thereof specifically binding to a polypeptide comprising an amino acid sequence selected from the group consisting of SEQ ID NOs: 21-22, 18-20 and 23-34, or comprising an agent selected from the group consisting of an antisense polynucleotide, a ribozyme, a small interfering RNA (siRNA), microRNA (miRNA) and a short-hairpin RNA (shRNA), wherein said agent comprises a nucleic acid sequence complementary to, or engineered from, a naturally-occurring polynucleotide sequence of about 17 to about 30 contiguous nucleotides of a nucleic acid sequence selected from the group consisting of SEQ ID NO: 4-5, 1-3 and 6-17.

33. The method according to claim 32 wherein said antagonist is a monoclonal antibody.

34. The method according to claim 32 wherein said antagonist is a single chain antibody.

35. (canceled)

36. The method according to claim 32, wherein said disease is a fibrotic disease or cancer.

37. The method according to claim 32, wherein said disease is selected from idiopathic pulmonary fibrosis (IPF), cystic fibrosis, other diffuse parenchymal lung diseases of different etiologies including iatrogenic drug-induced fibrosis, occupational and/or environmental induced fibrosis, granulomatous diseases (sarcoidosis, hypersensitivity pneumonia), collagen vascular disease, alveolar proteinosis, langerhans cell granulomatosis, lymphangioleiomyomatosis, inherited diseases (Hermansky-Pudlak Syndrome, tuberous sclerosis, neurofibromatosis, metabolic storage disorders, familial interstitial lung disease), radiation induced fibrosis, chronic obstructive pulmonary disease (COPD), scleroderma, bleomycin induced pulmonary fibrosis, chronic asthma, silicosis, asbestos induced pulmonary fibrosis, acute respiratory distress syndrome (ARDS), kidney fibrosis, tubulointerstitium fibrosis, glomerular nephritis, focal segmental glomerular sclerosis, IgA nephropathy, hypertension, Alport syndrome, gut fibrosis, liver fibrosis, cirrhosis, alcohol induced liver fibrosis, toxic/drug induced liver fibrosis, hemochromatosis, nonalcoholic steatohepatitis (NASH), biliary duct injury, primary biliary cirrhosis, infection induced liver fibrosis, viral induced liver fibrosis, autoimmune hepatitis, corneal scarring, hypertrophic scarring, Dupuytren disease, keloids, cutaneous fibrosis, cutaneous scleroderma, systemic sclerosis, spinal cord injury/fibrosis, myelofibrosis, vascular restenosis, atherosclerosis, arteriosclerosis, Wegener's granulomatosis and Peyronie's disease.

38. The method according to claim 32, wherein said disease is selected from melanoma, lymphoma, leukaemia, fibrosarcoma, rhabdomyosarcoma, mastocytoma, colorectal cancer, prostate cancer, small cell lung cancer and non-small cell lung cancer, breast cancer, pancreatic cancer, bladder cancer, renal cancer, gastric cancer, glioblastoma, primary liver cancer, ovarian cancer, prostate cancer and uterine leiomyosarcoma.

39. The method according to claim 32, wherein said disease is a cancer metastasis.

40. An in vitro method of inhibiting epithelial mesenchymal transition, comprising contacting a population of epithelial cells with an inhibitor of the activity and/or expression of a polypeptide comprising an amino acid sequence selected from the group consisting of SEQ ID NOs: 21-22, 18-20 and 23-34.

41. The method of claim 40 wherein said inhibitor is an antibody.

42. The method of claim 40 wherein said antibody is a monoclonal antibody.

43. The method of claim 40 wherein said inhibitor is selected from the group consisting of an antisense polynucleotide, a ribozyme, a small interfering RNA (siRNA), microRNA (miRNA) and a short-hairpin RNA (shRNA), wherein said inhibitor comprises a nucleic acid sequence complementary to, or engineered from, a naturally-occurring polynucleotide sequence of about 17 to about 30 contiguous nucleotides of a nucleic acid encoding said polypeptide.


SEQUENCE LISTING (78 sequences)

SEQ ID NO: 1 - DNA, 2175 bp, Homo sapiens
gctcacggcggcggcggcggagcggagaggccagagccggagaccgagctgggatcgggccccgggcgggggcggtgcgagcggcgccaagcagatcttaggggcggggacggagccggggcgggcgggactgaagcggagcccgggaacggggcgggaggtcccagggtcccgggttgggggggtggagcagcatttcgtcgccgcgggggtgccgggactccggccgcagtgtcgccgccatcacggacttcctgtgggacaagcgcacgggcctcgccgccagaacgatgccgcatcctcgaaggtaccactcctcagagcgaggcagccgggggagttaccgtgaacactatcggagccgaaagcataagcgacgaagaagtcgctcctggtcaagtagtagtgaccggacacgacggcgtcggcgagaggacagctaccatgtccgttctcgaagcagttatgatgatcgttcgtccgaccggagggtgtatgaccggcgatactgtggcagctacagacgcaacgattatagccgggatcggggagatgcctactatgacacagactatcggcattcctatgaatatcagcgggagaacagcagttaccgcagccagcgcagcagccggaggaagcacagacggcggaggaggcgcagccggacatttagccgctcatcttcgcacagcagccggagagccaagagtgtagaggacgacgctgagggccacctcatctaccacgtcggggactggctacaagagcgatatgaaatcgttagcaccttaggagaggggaccttcggccgagttgtacaatgtgttgaccatcgcaggggtggggctcgagttgccctgaagatcattaagaatgtggagaagtacaaggaagcagctcgacttgagatcaacgtgctagagaaaatcaatgagaaagaccctgacaacaagaacctctgtgtccagatgtttgactggtttgactaccatggccacatgtgtatctcctttgagcttctgggccttagcaccttcgatttcctcaaagacaacaactacctgccctaccccatccaccaagtgcgccacatggccttccagctgtgccaggctgtcaagttcctccatgataacaagctgacacatacagacctcaagcctgaaaatattctgtttgtgaattcagactatgagctcacctacaacctagagaagaagcgagatgagcgcagtgtgaagagcacagctgtgcgggtggtagactttggcagtgccacctttgaccatgagcaccatagcaccattgtctccactcgccattaccgagcaccagaagtcatccttgagttgggctggtcacagccttgtgatgtgtggagtataggctgcatcatctttgaatactatgtgggattcaccctcttccagacccatgacaacagagagcatctagccatgatggaaaggatcttgggtcctatcccttcccggatgatccgaaagacaagaaagcagaaatatttttaccggggtcgcctggattgggatgagaacacatcagctgggcgctatgttcgtgagaactgcaaaccgctgcggcggtatctgacctcagaggcagaggaacaccaccagctcttcgatctgattgaaagcatgctagagtatgaaccagctaagcggctgaccttgggtgaagcccttcagcatcctttcttcgcccgccttcgggctgagccgcccaacaagttgtgggactccagtcgggatatcagtcggtgacgatcaggccctgggcccccctgcatcttttatagcagtgggtgtccagtccaggacactggtgcttttttatacaagagaacgagccagagttcactccttcctcctggctctctatatacctgtgaatatgtgaaatagtgtaaatatgaaagaacttgtacctatcacttcaacccctgccttgtacataatactattccatccacacagtttccaccctcacctgccccctcatacggagttggatgggggccgagtgaggtaaccaggtggcatctaccccatgttttataaggaattttgtacagtctttgtgaaataaaataacgtgcttcatttgacccccaaaaaaaaaaaaaaaaaaaaaaaaaaaaaaaaaaaaaaa

SEQ ID NO: 2 - DNA, 1674 bp, Homo sapiens
gcggccgcccgccgccgcgctcctcctcctcctcctccagcgcccggcggcccgctgcctcctccgcccgacgccccgcgtcccccgccgcgccgccgccgccaccctctgcgccccgcgccgccccccggtcccgcccgccatgcccggcccggccgcgggcagcagggcccgggtctacgccgaggtgaacagtctgaggagccgcgagtactgggactacgaggctcacgtcccgagctggggtaatcaagatgattaccaactggttcgaaaacttggtcggggaaaatatagtgaagtatttgaggccattaatatcaccaacaatgagagagtggttgtaaaaatcctgaagccagtgaagaaaaagaagataaaacgagaggttaagattctggagaaccttcgtggtggaacaaatatcattaagctgattgacactgtaaaggaccccgtgtcaaagacaccagctttggtatttgaatatatcaataatacagattttaagcaactctaccagatcctgacagactttgatatccggttttatatgtatgaactacttaaagctctggattactgccacagcaagggaatcatgcacagggatgtgaaacctcacaatgtcatgatagatcaccaacagaaaaagctgcgactgatagattggggtctggcagaattctatcatcctgctcaggagtacaatgttcgtgtagcctcaaggtacttcaagggaccagagctcctcgtggactatcagatgtatgattatagcttggacatgtggagtttgggctgtatgttagcaagcatgatctttcgaagggaaccattcttccatggacaggacaactatgaccagcttgttcgcattgccaaggttctgggtacagaagaactgtatgggtatctgaagaagtatcacatagacctagatccacacttcaacgatatcctgggacaacattcacggaaacgctgggaaaactttatccatagtgagaacagacaccttgtcagccctgaggccctagatcttctggacaaacttctgcgatacgaccatcaacagagactgactgccaaagaggccatggagcacccatacttctaccctgtggtgaaggagcagtcccagccttgtgcagacaatgctgtgctttccagtggtctcacggcagcacgatgaagactggaaagcgacgggtctgttgcggttctcccacttttccataagcagaacaagaaccaaatcaaacgtcttaacgcgtatagagagatcacgttccgtgagcagacacaaaacggtggcaggtttggcgagcacgaactagaccaagcgaagggcagcccaccaccgtatatcaaacctcacttccgaatgtaaaaggctcacttgcctttggcttcctgttgacttcttcccgacccagaaagcatggggaatgtgaagggtatgcagaatgttgttggttactgttgctccccgagcccctcaactcgtcccgtggccgcctgtttttccagcaaaccacgctaactagctgaccacagactccacagtggggggacgggcgcagtatgtggcatggcggcagttacatattattattttaaaagtatatattattgaataaaaggttttaaaagaaaaaaaaaaaaaaaaaa

SEQ ID NO: 3 - DNA, 4001 bp, Homo sapiens
aggcatcagcaatctatcagggaacggcggtggccggtgcggcgtgttcggtggcggctctggccgctcaggcgcctgcggctgggtgagcgcacgcgaggcggcgaggcggcagcgtgtttctaggtcgtggcgtcgggcttccggagctttggcggcagctaggggaggatggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaaacaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcacttcatgaaattatatgaagaaaaaaccgggaacgcttggcactccaaaaatttcacgaagtatcccaaaaagttctaccccctggagattgactatggccaggatgaagaggcagtgaagaagctgacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacaatgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactgccatacgtctcagggagacccaataggcttaatcctgttgggagaagttgcccttggaaacatgtatgaactgaagcacgcttcacatatcagcaagttacccaagggcaagcacagtgtcaaaggtttgggcaaaactacccctgatccttcagctaacattagtctggatggtgtagacgttcctcttgggaccgggatttcatctggtgtgaatgacacctctctactatataacgagtacattgtctatgatattgctcaggtaaatctgaagtatctgctgaaactgaaattcaattttaagacctccctgtggtaattgggagaggtagccgagtcacacccggtggctctggtatgaattcacccgaagcgcttctgcaccaactcacctggccgctaagttgctgatgggtagtacctgtactaaaccacctcagaaaggattttacagaaacgtgttaaaggttttctctaacttctcaagtcccttgttttgtgttgtgtctgtggggaggggttgttttggggttgtttttgttttttcttgccaggtagataaaactgacatagagaaaaggctggagagagattctgttgcatagactagtcctatggaaaaaaccaagcttcgttagaatgtctgccttactggtttccccagggaaggaaaaatacacttccacccttttttctaagtgttcgtctttagttttgattttggaaagatgttaagcatttatttttagttaaaaataaaaactaatttcatactatttagattttcttttttatcttgcacttattgtcccctttttagttttttttgtttgcctcttgtggtgaggggtgtgggaagaccaaaggaaggaacgctaacaatttctcatacttagaaacaaaaagagctttccttctccaggaatactgaacatgggagctcttgaaatatgtagtattaaaagttgcatttgaaattcttgactttcttatgggcacttttgtcttccaaattaaaactctaccacaaatatacttacccaagggctaatagtaatactcgattaaaaatgcagatgccttctctaaaaaaaaaaaaaaaaa

SEQ ID NO: 4 - DNA, 1137 bp, Homo sapiens
actcgcgcccttgccgctgccaccgcaccccgccatggagcggccgtcgctgcgcgccctgctcctcggcgccgctgggctgctgctcctgctcctgcccctctcctcttcctcctcttcggacacctgcggcccctgcgagccggcctcctgcccgcccctgcccccgctgggctgcctgctgggcgagacccgcgacgcgtgcggctgctgccctatgtgcgcccgcggcgagggcgagccgtgcgggggtggcggcgccggcagggggtactgcgcgccgggcatggagtgcgtgaagagccgcaagaggcggaagggtaaagccggggcagcagccggcggtccgggtgtaagcggcgtgtgcgtgtgcaagagccgctacccggtgtgcggcagcgacggcaccacctacccgagcggctgccagctgcgcgccgccagccagagggccgagagccgcggggagaaggccatcacccaggtcagcaagggcacctgcgagcaaggtccttccatagtgacgccccccaaggacatctggaatgtcactggtgcccaggtgtacttgagctgtgaggtcatcggaatcccgacacctgtcctcatctggaacaaggtaaaaaggggtcactatggagttcaaaggacagaactcctgcctggtgaccgggacaacctggccattcagacccggggtggcccagaaaagcatgaagtaactggctgggtgctggtatctcctctaagtaaggaagatgctggagaatatgagtgccatgcatccaattcccaaggacaggcttcagcatcagcaaaaattacagtggttgatgccttacatgaaataccagtgaaaaaaggtgaaggtgccgagctataaacctccagaatattattagtctgcatggttaaaagtagtcatggataactacattacctgttcttgcctaataagtttcttttaatccaatccactaacactttagttatattcactggttttacacagagaaatacaaaataaagatcacacatcaagactatctacaaaaatttattatatatttacagaagaaaagcatgcatatcattaaacaaataaaatactttttatcacaacacagtaaaaaaaaa

SEQ ID NO: 5 - DNA, 1051 bp, Homo sapiens
actcgcgcccttgccgctgccaccgcaccccgccatggagcggccgtcgctgcgcgccctgctcctcggcgccgctgggctgctgctcctgctcctgcccctctcctcttcctcctcttcggacacctgcggcccctgcgagccggcctcctgcccgcccctgcccccgctgggctgcctgctgggcgagacccgcgacgcgtgcggctgctgccctatgtgcgcccgcggcgagggcgagccgtgcgggggtggcggcgccggcagggggtactgcgcgccgggcatggagtgcgtgaagagccgcaagaggcggaagggtaaagccggggcagcagccggcggtccgggtgtaagcggcgtgtgcgtgtgcaagagccgctacccggtgtgcggcagcgacggcaccacctacccgagcggctgccagctgcgcgccgccagccagagggccgagagccgcggggagaaggccatcacccaggtcagcaagggcacctgcgagcaaggtccttccatagtgacgccccccaaggacatctggaatgtcactggtgcccaggtgtacttgagctgtgaggtcatcggaatcccgacacctgtcctcatctggaacaaggtaaaaaggggtcactatggagttcaaaggacagaactcctgcctggtgaccgggacaacctggccattcagacccggggtggcccagaaaagcatgaagtaactggctgggtgctggtatctcctctaagtaaggaagatgctggagaatatgagtgccatgcatccaattcccaaggacaggcttcagcatcagcaaaaattacagtggttgatgccttacatgaaataccagtgaaaaaaggtacacaataaatctcacagccatttaaaaatgactagtacatttgctttaaaaagaacagaactaagtatgaaagtatcagacgtagctattgatgaaattctgtagttagcaacccataagggcattaagtatgccattaaaatgtacagcatgagactccaaaagattatctggatgggtgactg

SEQ ID NO: 6 - DNA, 2885 bp, Homo sapiens
gactttcactttccctttcgaattcctcggtatatcttggggactggaggacctgtctggttattatacagacgcataactggaggtgggatccacacagctcagaacagctggatcttgctcagtctctgccaggggaagattccttggaggaggccctgcagcgacatggagggagctgctttgctgagagtctctgtcctctgcatctggatgagtgcacttttccttggtgtgggagtgagggcagaggaagctggagcgagggtgcaacaaaacgttccaagtgggacagatactggagatcctcaaagtaagcccctcggtgactgggctgctggcaccatggacccagagagcagtatctttattgaggatgccattaagtatttcaaggaaaaagtgagcacacagaatctgctactcctgctgactgataatgaggcctggaacggattcgtggctgctgctgaactgcccaggaatgaggcagatgagctccgtaaagctctggacaaccttgcaagacaaatgatcatgaaagacaaaaactggcacgataaaggccagcagtacagaaactggtttctgaaagagtttcctcggttgaaaagtgagcttgaggataacataagaaggctccgtgcccttgcagatggggttcagaaggtccacaaaggcaccaccatcgccaatgtggtgtctggctctctcagcatttcctctggcatcctgaccctcgtcggcatgggtctggcacccttcacagagggaggcagccttgtactcttggaacctgggatggagttgggaatcacagccgctttgaccgggattaccagcagtaccatggactacggaaagaagtggtggacacaagcccaagcccacgacctggtcatcaaaagccttgacaaattgaaggaggtgagggagtttttgggtgagaacatatccaactttctttccttagctggcaatacttaccaactcacacgaggcattgggaaggacatccgtgccctcagacgagccagagccaatcttcagtcagtaccgcatgcctcagcctcacgcccccgggtcactgagccaatctcagctgaaagcggtgaacaggtggagagggttaatgaacccagcatcctggaaatgagcagaggagtcaagctcacggatgtggcccctgtaagcttctttcttgtgctggatgtagtctacctcgtgtacgaatcaaagcacttacatgagggggcaaagtcagagacagctgaggagctgaagaaggtggctcaggagctggaggagaagctaaacattctcaacaataattataagattctgcaggcggaccaagaactgtgaccacagggcagggcagccaccaggagagatatgcctggcaggggccaggacaaaatgcaaacttttttttttttctgagacagagtcttgctctgtcgccaagttggagtgcaatggtgcgatctcagctcactgcaagctctgcctcccgtgttcaagcgattctcctgccttggcctcccaagtagctgggactacaggcgcctaccaccatgcccagctaatttttgtatttttaatagagatggggtttcaccatgttggccaggatggtctcgatctcctgacctcttgatctgcccaccttggcctcccaaagtgctgggattacaggcgtgagccatcgcttttgacccaaatgcaaacattttattagggggataaagagggtgaggtaaagtttatggaactgagtgttagggactttggcatttccatagctgagcacagcaggggaggggttaatgcagatggcagtgcagcaaggagaaggcaggaacattggagcctgcaataagggaaaaatgggaactggagagtgtggggaatgggaagaagcagtttactttagactaaagaatatattggggggccgggtgtagtggctcatgcctgtaatccgagcactttgggaggccaaggcgggcggatcacgaggtcaggagatcgagaccatcctggctaacacagtgaaaccccgtctctactaaaaatacaaaaaattagccgggcatggtggcgggcgcctgtagttccagctaactgggcggctgaggcaggagaatggcgtgaacctgggaggtggagcttgcagtgagccgagatatcgccactgcactccagcctgggtgacagagcgagactccatctcaaaaaaaaaaaaaaaaagaatatattgacggaagaatagagaggaggcttgaaggaaccagcaatgagaaggccaggaaaagaaagagctgaaaatggagaaagcccaagagttagaacagttggatacaggagaagaaacagcggctccactacagacccagccccaggttcaatgtcctccgaagaatgaagtctttccctggtgatggtcccctgccctgtctttccagcatccactctcccttgtcctcctgggggcatatctcagtcaggcagcggcttcctgatgatggtcattggggtggttgtcatgtgatgggtcccctccaggttactaaagggtgcatgtcccctgcttgaacactgaagggcaggtggtgggccatggccatggtccccagctgaggagcaggtgtccctgagaacccaaacttcccagagagtatgtgagaaccaaccaatgaaaacagtcccatcgctcttacccggtaagtaaacagtcagaaaattagcatgaaagcagtttagcattgggaggaagctcagatctctagagctgtcttgtcgccgcccaggattgacctgtgtgtaagtcccaataaactcacctactcatcaagctggaaaaaaaaaaaaaaaaaaaaaaa

SEQ ID NO: 7 - DNA, 3039 bp, Homo sapiens
gactttcactttccctttcgaattcctcggtatatcttggggactggaggacctgtctggttattatacagacgcataactggaggtgggatccacacagctcagaacagctggatcttgctcagtctctgccaggggaagattccttgacttctggggtgatggagaagaaacaggctgtgctgtgtccctaatgggaaacgtggctgagacaggggagtgagaagggtgcgttgcagaatggtgcctgtggcatgatgccagctttgcaatcatgagattcaaaagccacactgtggaattgaggaggccctgcagcgacatggagggagctgctttgctgagagtctctgtcctctgcatctggatgagtgcacttttccttggtgtgggagtgagggcagaggaagctggagcgagggtgcaacaaaacgttccaagtgggacagatactggagatcctcaaagtaagcccctcggtgactgggctgctggcaccatggacccagagagcagtatctttattgaggatgccattaagtatttcaaggaaaaagtgagcacacagaatctgctactcctgctgactgataatgaggcctggaacggattcgtggctgctgctgaactgcccaggaatgaggcagatgagctccgtaaagctctggacaaccttgcaagacaaatgatcatgaaagacaaaaactggcacgataaaggccagcagtacagaaactggtttctgaaagagtttcctcggttgaaaagtgagcttgaggataacataagaaggctccgtgcccttgcagatggggttcagaaggtccacaaaggcaccaccatcgccaatgtggtgtctggctctctcagcatttcctctggcatcctgaccctcgtcggcatgggtctggcacccttcacagagggaggcagccttgtactcttggaacctgggatggagttgggaatcacagccgctttgaccgggattaccagcagtaccatggactacggaaagaagtggtggacacaagcccaagcccacgacctggtcatcaaaagccttgacaaattgaaggaggtgagggagtttttgggtgagaacatatccaactttctttccttagctggcaatacttaccaactcacacgaggcattgggaaggacatccgtgccctcagacgagccagagccaatcttcagtcagtaccgcatgcctcagcctcacgcccccgggtcactgagccaatctcagctgaaagcggtgaacaggtggagagggttaatgaacccagcatcctggaaatgagcagaggagtcaagctcacggatgtggcccctgtaagcttctttcttgtgctggatgtagtctacctcgtgtacgaatcaaagcacttacatgagggggcaaagtcagagacagctgaggagctgaagaaggtggctcaggagctggaggagaagctaaacattctcaacaataattataagattctgcaggcggaccaagaactgtgaccacagggcagggcagccaccaggagagatatgcctggcaggggccaggacaaaatgcaaacttttttttttttctgagacagagtcttgctctgtcgccaagttggagtgcaatggtgcgatctcagctcactgcaagctctgcctcccgtgttcaagcgattctcctgccttggcctcccaagtagctgggactacaggcgcctaccaccatgcccagctaatttttgtatttttaatagagatggggtttcaccatgttggccaggatggtctcgatctcctgacctcttgatctgcccaccttggcctcccaaagtgctgggattacaggcgtgagccatcgcttttgacccaaatgcaaacattttattagggggataaagagggtgaggtaaagtttatggaactgagtgttagggactttggcatttccatagctgagcacagcaggggaggggttaatgcagatggcagtgcagcaaggagaaggcaggaacattggagcctgcaataagggaaaaatgggaactggagagtgtggggaatgggaagaagcagtttactttagactaaagaatatattggggggccgggtgtagtggctcatgcctgtaatccgagcactttgggaggccaaggcgggcggatcacgaggtcaggagatcgagaccatcctggctaacacagtgaaaccccgtctctactaaaaatacaaaaaattagccgggcatggtggcgggcgcctgtagttccagctaactgggcggctgaggcaggagaatggcgtgaacctgggaggtggagcttgcagtgagccgagatatcgccactgcactccagcctgggtgacagagcgagactccatctcaaaaaaaaaaaaaaaaagaatatattgacggaagaatagagaggaggcttgaaggaaccagcaatgagaaggccaggaaaagaaagagctgaaaatggagaaagcccaagagttagaacagttggatacaggagaagaaacagcggctccactacagacccagccccaggttcaatgtcctccgaagaatgaagtctttccctggtgatggtcccctgccctgtctttccagcatccactctcccttgtcctcctgggggcatatctcagtcaggcagcggcttcctgatgatggtcattggggtggttgtcatgtgatgggtcccctccaggttactaaagggtgcatgtcccctgcttgaacactgaagggcaggtggtgggccatggccatggtccccagctgaggagcaggtgtccctgagaacccaaacttcccagagagtatgtgagaaccaaccaatgaaaacagtcccatcgctcttacccggtaagtaaacagtcagaaaattagcatgaaagcagtttagcattgggaggaagctcagatctctagagctgtcttgtcgccgcccaggattgacctgtgtgtaagtcccaataaactcacctactcatcaagctggaaaaaaaaaaaaaaaaaaaaaaa

SEQ ID NO: 8 - DNA, 2924 bp, Homo sapiens
gactttcactttccctttcgaattcctcggtatatcttggggactggaggacctgtctggttattatacagacgcataactggaggtgggatccacacagctcagaacagctggatcttgctcagtctctgccaggggaagattccttggaggagcacactgtctcaacccctcttttcctgctcaaggaggaggccctgcagcgacatggagggagctgctttgctgagagtctctgtcctctgcatctggatgagtgcacttttccttggtgtgggagtgagggcagaggaagctggagcgagggtgcaacaaaacgttccaagtgggacagatactggagatcctcaaagtaagcccctcggtgactgggctgctggcaccatggacccagagagcagtatctttattgaggatgccattaagtatttcaaggaaaaagtgagcacacagaatctgctactcctgctgactgataatgaggcctggaacggattcgtggctgctgctgaactgcccaggaatgaggcagatgagctccgtaaagctctggacaaccttgcaagacaaatgatcatgaaagacaaaaactggcacgataaaggccagcagtacagaaactggtttctgaaagagtttcctcggttgaaaagtgagcttgaggataacataagaaggctccgtgcccttgcagatggggttcagaaggtccacaaaggcaccaccatcgccaatgtggtgtctggctctctcagcatttcctctggcatcctgaccctcgtcggcatgggtctggcacccttcacagagggaggcagccttgtactcttggaacctgggatggagttgggaatcacagccgctttgaccgggattaccagcagtaccatggactacggaaagaagtggtggacacaagcccaagcccacgacctggtcatcaaaagccttgacaaattgaaggaggtgagggagtttttgggtgagaacatatccaactttctttccttagctggcaatacttaccaactcacacgaggcattgggaaggacatccgtgccctcagacgagccagagccaatcttcagtcagtaccgcatgcctcagcctcacgcccccgggtcactgagccaatctcagctgaaagcggtgaacaggtggagagggttaatgaacccagcatcctggaaatgagcagaggagtcaagctcacggatgtggcccctgtaagcttctttcttgtgctggatgtagtctacctcgtgtacgaatcaaagcacttacatgagggggcaaagtcagagacagctgaggagctgaagaaggtggctcaggagctggaggagaagctaaacattctcaacaataattataagattctgcaggcggaccaagaactgtgaccacagggcagggcagccaccaggagagatatgcctggcaggggccaggacaaaatgcaaacttttttttttttctgagacagagtcttgctctgtcgccaagttggagtgcaatggtgcgatctcagctcactgcaagctctgcctcccgtgttcaagcgattctcctgccttggcctcccaagtagctgggactacaggcgcctaccaccatgcccagctaatttttgtatttttaatagagatggggtttcaccatgttggccaggatggtctcgatctcctgacctcttgatctgcccaccttggcctcccaaagtgctgggattacaggcgtgagccatcgcttttgacccaaatgcaaacattttattagggggataaagagggtgaggtaaagtttatggaactgagtgttagggactttggcatttccatagctgagcacagcaggggaggggttaatgcagatggcagtgcagcaaggagaaggcaggaacattggagcctgcaataagggaaaaatgggaactggagagtgtggggaatgggaagaagcagtttactttagactaaagaatatattggggggccgggtgtagtggctcatgcctgtaatccgagcactttgggaggccaaggcgggcggatcacgaggtcaggagatcgagaccatcctggctaacacagtgaaaccccgtctctactaaaaatacaaaaaattagccgggcatggtggcgggcgcctgtagttccagctaactgggcggctgaggcaggagaatggcgtgaacctgggaggtggagcttgcagtgagccgagatatcgccactgcactccagcctgggtgacagagcgagactccatctcaaaaaaaaaaaaaaaaagaatatattgacggaagaatagagaggaggcttgaaggaaccagcaatgagaaggccaggaaaagaaagagctgaaaatggagaaagcccaagagttagaacagttggatacaggagaagaaacagcggctccactacagacccagccccaggttcaatgtcctccgaagaatgaagtctttccctggtgatggtcccctgccctgtctttccagcatccactctcccttgtcctcctgggggcatatctcagtcaggcagcggcttcctgatgatggtcattggggtggttgtcatgtgatgggtcccctccaggttactaaagggtgcatgtcccctgcttgaacactgaagggcaggtggtgggccatggccatggtccccagctgaggagcaggtgtccctgagaacccaaacttcccagagagtatgtgagaaccaaccaatgaaaacagtcccatcgctcttacccggtaagtaaacagtcagaaaattagcatgaaagcagtttagcattgggaggaagctcagatctctagagctgtcttgtcgccgcccaggattgacctgtgtgtaagtcccaataaactcacctactcatcaagctggaaaaaaaaaaaaaaaaaaaaaaa

SEQ ID NO: 9 - DNA, 2831 bp, Homo sapiens
gactttcactttccctttcgaattcctcggtatatcttggggactggaggacctgtctggttattatacagacgcataactggaggtgggatccacacagctcagaacagctggatcttgctcagtctctgccaggggaagattccttggaggaggccctgcagcgacatggagggagctgctttgctgagagtctctgtcctctgcatctgggtgcaacaaaacgttccaagtgggacagatactggagatcctcaaagtaagcccctcggtgactgggctgctggcaccatggacccagagagcagtatctttattgaggatgccattaagtatttcaaggaaaaagtgagcacacagaatctgctactcctgctgactgataatgaggcctggaacggattcgtggctgctgctgaactgcccaggaatgaggcagatgagctccgtaaagctctggacaaccttgcaagacaaatgatcatgaaagacaaaaactggcacgataaaggccagcagtacagaaactggtttctgaaagagtttcctcggttgaaaagtgagcttgaggataacataagaaggctccgtgcccttgcagatggggttcagaaggtccacaaaggcaccaccatcgccaatgtggtgtctggctctctcagcatttcctctggcatcctgaccctcgtcggcatgggtctggcacccttcacagagggaggcagccttgtactcttggaacctgggatggagttgggaatcacagccgctttgaccgggattaccagcagtaccatggactacggaaagaagtggtggacacaagcccaagcccacgacctggtcatcaaaagccttgacaaattgaaggaggtgagggagtttttgggtgagaacatatccaactttctttccttagctggcaatacttaccaactcacacgaggcattgggaaggacatccgtgccctcagacgagccagagccaatcttcagtcagtaccgcatgcctcagcctcacgcccccgggtcactgagccaatctcagctgaaagcggtgaacaggtggagagggttaatgaacccagcatcctggaaatgagcagaggagtcaagctcacggatgtggcccctgtaagcttctttcttgtgctggatgtagtctacctcgtgtacgaatcaaagcacttacatgagggggcaaagtcagagacagctgaggagctgaagaaggtggctcaggagctggaggagaagctaaacattctcaacaataattataagattctgcaggcggaccaagaactgtgaccacagggcagggcagccaccaggagagatatgcctggcaggggccaggacaaaatgcaaacttttttttttttctgagacagagtcttgctctgtcgccaagttggagtgcaatggtgcgatctcagctcactgcaagctctgcctcccgtgttcaagcgattctcctgccttggcctcccaagtagctgggactacaggcgcctaccaccatgcccagctaatttttgtatttttaatagagatggggtttcaccatgttggccaggatggtctcgatctcctgacctcttgatctgcccaccttggcctcccaaagtgctgggattacaggcgtgagccatcgcttttgacccaaatgcaaacattttattagggggataaagagggtgaggtaaagtttatggaactgagtgttagggactttggcatttccatagctgagcacagcaggggaggggttaatgcagatggcagtgcagcaaggagaaggcaggaacattggagcctgcaataagggaaaaatgggaactggagagtgtggggaatgggaagaagcagtttactttagactaaagaatatattggggggccgggtgtagtggctcatgcctgtaatccgagcactttgggaggccaaggcgggcggatcacgaggtcaggagatcgagaccatcctggctaacacagtgaaaccccgtctctactaaaaatacaaaaaattagccgggcatggtggcgggcgcctgtagttccagctaactgggcggctgaggcaggagaatggcgtgaacctgggaggtggagcttgcagtgagccgagatatcgccactgcactccagcctgggtgacagagcgagactccatctcaaaaaaaaaaaaaaaaagaatatattgacggaagaatagagaggaggcttgaaggaaccagcaatgagaaggccaggaaaagaaagagctgaaaatggagaaagcccaagagttagaacagttggatacaggagaagaaacagcggctccactacagacccagccccaggttcaatgtcctccgaagaatgaagtctttccctggtgatggtcccctgccctgtctttccagcatccactctcccttgtcctcctgggggcatatctcagtcaggcagcggcttcctgatgatggtcattggggtggttgtcatgtgatgggtcccctccaggttactaaagggtgcatgtcccctgcttgaacactgaagggcaggtggtgggccatggccatggtccccagctgaggagcaggtgtccctgagaacccaaacttcccagagagtatgtgagaaccaaccaatgaaaacagtcccatcgctcttacccggtaagtaaacagtcagaaaattagcatgaaagcagtttagcattgggaggaagctcagatctctagagctgtcttgtcgccgcccaggattgacctgtgtgtaagtcccaataaactcacctactcatcaagctggaaaaaaaaaaaaaaaaaaaaaaa

SEQ ID NO: 10 - DNA, 6344 bp, Homo sapiens
gcggaagtgtgggagggtctgcggggcgggctcaggaggtccgcgggaggatggagcagtgagcgggtctgggcggctgctggcagcgccatggagacggtacagctgaggaacccgccgcgccggcagctgaaaaagttggatgaagatagtttaaccaaacaaccagaagaagtatttgatgtcttagagaaacttggagaagggtcctatggcagcgtatacaaagctattcataaagagaccggccagattgttgctattaagcaagttcctgtggaatcagacctccaggagataatcaaagaaatctctataatgcagcaatgtgacagccctcatgtagtcaaatattatggcagttattttaagaacacagacttatggatcgttatggagtactgtggggctggttctgtatctgatatcattcgattacgaaataaaacgttaacagaagatgaaatagctacaatattacaatcaactcttaagggacttgaataccttcattttatgagaaaaatacaccgagatatcaaggcaggaaatattttgctaaatacagaaggacatgcaaaacttgcagattttggggtagcaggtcaacttacagataccatggccaagcggaatacagtgataggaacaccattttggatggctccagaagtgattcaggaaattggatacaactgtgtagcagacatctggtccctgggaataactgccatagaaatggctgaaggaaagcccccttatgctgatatccatccaatgagggcaatcttcatgattcctacaaatcctcctcccacattccgaaaaccagagctatggtcagataactttacagattttgtgaaacagtgtcttgtaaagagccctgagcagagggccacagccactcagctcctgcagcacccatttgtcaggagtgccaaaggagtgtcaatactgcgagacttaattaatgaagccatggatgtgaaactgaaacgccaggaatcccagcagcgggaagtggaccaggacgatgaagaaaactcagaagaggatgaaatggattctggcacgatggttcgagcagtgggtgatgagatgggcactgtccgagtagccagcaccatgactgatggagccaatactatgattgagcacgatgacacgttgccatcacaactgggcaccatggtgatcaatgcagaggatgaggaagaggaaggaactatgaaaagaagggatgagaccatgcagcctgcgaaaccatcctttcttgaatattttgaacaaaaagaaaaggaaaaccagatcaacagctttggcaagagtgtacctggtccactgaaaaattcttcagattggaaaataccacaggatggagactacgagtttcttaagagttggacagtggaggaccttcagaagaggctcttggccctggaccccatgatggagcaggagattgaagagatccggcagaagtaccagtccaagcggcagcccatcctggatgccatagaggctaagaagagacggcaacaaaacttctgagcaaggccaggctgtgagggccccagctccacccaggctttgggtgaattctggatggcttgcctcatgtttgttagccagcacttctgctctgtcgtctctccacagcacctttgtgaactcaggaatgtgcgccagtgggaagggctctcttgacagtcagcgtgccatcttgatgtgtgtatgtacattggtcaggtatattatctcaaaggatttatattggcgcttttaactcagagttttaaaccccaggaacagagactcctagttgagtgatagctgggaaagttttacattgtctgtttttcttctcccaatagctttcaattgttctttctggaagacttttaaaaaaatataaatatgcatatatatatataaattataaatagattccccacgcagtgtggtggcatctctgtacaggtacagttttaaacggtttgcctcttttctgtaagattatggtactgtggaacatgagggcagaggacaccgggaggctgttagggggtcactgaatcccaggagccaacctccccctttgcagggctgcatttaaaaattaggtttgggacagttcttgtaccgtggtttcagccttgtgtggtcatcactggcttctggagctattggtgatgtccaagggaaagctttgagagtttatgtttactctttgagtcccaggagaagcctggcaccctctttgcaaattggcctttgctctttcaatgcctttcatccatctccactctctcaactgcctaaagtcacagcacagatactgcccagtgccttaagaggagacatgatctctaccagggactctcagcaaacacgggactgtgttcagtccacaaaggaaaagcgtttttgaagctctcattgttcatgtaaaaatcatacacgtggcatgttgctccacattccttacacacaggggtagaggggattgcttttgtgacccacgttcaaatatgtgactgttttcttttctcttttactgctaagcagcctggaaaggataaatgaatattagactaagatttgttttccaggaggctcaatctgaacacacagaatgtcagagctggaagggactatagagatcatctgatctgatcctcttgtacggatgatcgcaaaactgaggtgtagagaggggaatggccaaaatcacaaagcaagttagcgttaagagctgagactagaattcagggtcctcactcccaggccaccgaaccatgcagccccttctttgggggaagagacctgtgtcagtcttggttaattgttccagggaaccttgctaacagaaacttgctcttgccttggctcttcagtagatgacctggctgtaaagagattccctggacgagccagatcattcagtttcagcgagtccttgagctccacaacatctaccagatatagcagacaagcacccatggaggcaggtttcgggcctgaagcagatcagagggctttgcaaaagacagcatagagccatcttcctgcaactttacctctttccctcagatggggagccatgactgggttgcacctcaggatactgtaatttgactccataattgcttttgctcctgaaacctgggaatcaatggaaaggcagggaatgtgcctcttctgtggccagattctgttatttgcaattaaagcaagtttttaaaaaatgcaagaggcagttgttagtcttcagggcttggcaactgaaatagctatgtggcggatacggaaaacagaggacaatttgaggatcttgctggaataataaatgacagctaccatttgttgagcacctattatatatcaggcactgagctgggtaggctctaaacttcacaataaccctgtgacttaactactttatctccattttgtagttgaagaaataagttcagagagaaagattccttcccaaggtcatgcagctagtaaatgatagaatcaggattcatagcatcactatagggggtcaatatttacacaaaaaaggaaagtcacaagcctgtttaaaatgaagtgaccaccttttcttgcatagactaaataactcgaactggcatttttaggttggaaagacagctgaattagtagttaagtctgatagccaagtaagttttaaaaaccaaagcatccaggatgcacacccctgcaccatttgctgtgcgaattaatagttctgtctctctctctctttcttttttctttttattctttgagatggattttcgctcttgtcgcccaggctggagtacaatggcacgatcttggctcactgcaacctccgcctcccgggttcaagcgattcttctgctgggattacagcatatgccaccatgcccagattatttttttgtatttgtagtagagacggggtttcaccatgtcagtcaggctggtcttgaactcctgacctcaggtgatccacccgcctcagcctcccacactgctgggattacaggcatgagccaccgctcctggcctctctttcttttttaaacaaagaactttgcacttggccagagaggaggagaaagcccattttctcccttcctaagctagatccaaataaaagaaagttcagttttcccccataactattcttgggtcatgaactttgatctggagtttgttttgtttcaggaatgtgtgcacccagcttgctgatccaacaaagtctattgcttaccagtctagcttgatgaagccttttggccagaagtcaatttgttttggatcagagaaatttcctgacaaggtatatttgttttctagtgacagaaaggcaaaggaacaagtcctagttgttgttgttgttgttgaatactaaatttaagatatgtcagcttgctttcaatgagccttgggcttctgttattgcttgagcatttggaactcgagcttccagagaaatttgaggtcctcgcttgttctctgccttcaagaaacaatgacctgattctgtctttaaaaaaaaaaatctcagaattctttttttgtttgtgtttttttttttttttgagacagagtctcactctgttgcccaggctggagtgcagtggcgccatctcggctcactgcaacctccgcctcccaggttcaagcaattctcctgcctcagcctcccaggtagctgccactacaggtgctgcaccaccacgcccggctaatttttgtatttttagtagagacagggtttcaccatattagccaggtgggtcttgaactcctgaccttgtgatccacccgcctcggcctcccaaagtgctgggattacaggcgtgagccaccttgcctggccaaaaatctcagaattctttaagactgttttaattgctccatcagtaattttgaagcactttcctttttttttttttttcccctttttgtccctttccccaagccaccaattggatggatgaatgtttgacggggaagaggaagggtaggaggatgcatggatgagtggatgagtggatcgatggatgtattgataaatagatagaaccagtcatctgaagcaacttaagaattgtagccttgactccttgagactgtagatttcgatccaggaaacatttatttagcacctgccagatgccagaaatttataccatttaaaactcagtaagtcttttaaatatcaggaaggagagaagcgacatcatgatacatcctatgggtattaaaaagccaatagaatattatgaataattttatgctaataaatttaacaacttcaacatcataaacaaattccttgaaaaataaaaagtaccaaaattcattcaagaagaaatagataccagcctgagcaacatggcaaaatcccatctctacaaaacatcaaaaaaaaaaaaaattagtcgggcatggtggtgcacacctgtaatcccagcttgtcaggaggctgaagtgggaggatcacctgagcccagggaggtcaaggatgcagtgagccatggtctcaccactgcactctagcctgggtgacagaatgagaccccgtctcaaaaaaaaagaagaagtagataatctgaatagccctatatctatagaaacttaatagtgctgggagatataggtattattatcctcattttacagatgtgaaaattgaggctcagagaagtaaagtctattgctcaaggtcatgtggctagaatatggcagagccatgattcagatccaggtcttctgattcttattccagtgtcctttctagcataccatgttgcctctaaagattgcagctccttatttactagaaaattgttcctgcccaatctacatctccacctcaccccatcttttcttaagcactatgtttgtgtttttatcagtattatattcattgtctttggaatacatgttcttgtttgtgtttggaaaaaaaatctcttttaccagcttgcactcggaccaacttggaaaaaaaaaagcttaaatgtttttgctatgtacagtttaaaaatgtgaagtttgtagctttaactttttgtaagaaaatctaataacactggcttaagtgctgacttgaaatgctattttgtaaggtttggatgtaagtaatcaattgaggtcagcagtttgtatgagacatagcttcctccattgcccccactccttttttcttttttaagtttgagatgcttcctgtgtttttatgttagaattgttgttctccttcttttcttcttcctatacctcatcacgtttgttttaaataaactgtcctttggaccacaaaaaaaaaaaaaaaa

SEQ ID NO: 11 - DNA, 3306 bp, Homo sapiens
gcgcacgcgcagcaccccatttaagtttctcgtctttgcagtggctttgcttagatccggtgccgccttgaaggcggggctgggtcccagccgtagccaatggagccccgggtgagggttgaggggtggaaggtgcctactagccggtgcaggtttcttctagcgcgtgtgctggggtacctggtcgtcatggaggcggtattgaccgaagagcttgatgaggaagagcagctgctgagaaggcatcgcaaagagaagaaggagttgcaagccaaaattcagggcatgaagaatgctgttcccaagaatgacaagaagaggaggaagcaactcaccgaagatgtggccaagttggaaaaagaaatggaacagaaacatagagaggaactggagcaattgaagctgactactaaggagaataagatagattctgttgctgttaacatttcaaacttggtgcttgagaatcagccacctcggatatcaaaagcacaaaagagacgggaaaagaaagctgcattggaaaaggagcgagaagaacggatagctgaagctgaaattgaaaacttaacaggagccagacatatggaaagtgagaaacttgctcaaatattggcagctagacagttagaaattaaacagattccatctgatggccactgtatgtataaagccattgaagatcaactgaaagaaaaggattgtgctctgactgtggttgccttgagaagtcagaccgctgagtatatgcaaagccatgtggaagactttctgccatttttaacaaaccctaatacaggagatatgtatactccagaagaatttcagaagtactgtgaagatattgtaaacacagctgcatggggaggtcagcttgagctaagagctctgtctcacattttacaaacaccaatagagataatacaggcagattctcctcccattatagttggtgaagaatattcaaaaaaaccactaatacttgtatatatgagacatgcatatggcttaggagaacattataattcggttacacggttggtaaacatagttactgaaaattgcagctaatttatacaatgttgtacaattatgttttaatacagtgtgctgaactgagtatttctaccaagtgttgggttgttctaaatgctactgaaaaacacaactaccttatatcagttttatggcaaagctactaacaggtgtttttagaaatatgtcagagataaactttaaccagtgtcttcttagtggaattttaaaaatttgttaagttcattgtagagaacaccattcatagaccaagatggtcccctattagctgatattttcatttatgtaagatcctggacattctgttttgtgttggaacaaattttcaatgtttttaattctcccttttctgcctgttcctaaaaactttcaaaataaccatttcaatgtaatttgtcttgaagaagtttacccaatatctttgtcattgcaataatgtagtatggtgatggtcaatttaattattgcttataaaatgaacttgattgcagtaaccatggtatcgatgagactaatgcagtgaagctttattactaatatatatagccttatcaaagcaagctaagaagcttgctttaataattaataatttaaaaatatttatttagatgggattcagttgtttaaagaagtgagataaattcaaaggtaaataggtattctctgtcaaatttaaatctctaaagaattagagattagttttttatttgtgtataatttaaaaaagacctaataggttactgtctttttaatgcatttgttattctttcgtattttttaagcatcggactggagttagtaccatcatctcttgagtctttcataactacacatttttatatttccttttgtgttttctactttgctttccttgaaggaacaagcattttgtggcctttaccttgtgcaattttagggagtagcagtgtaaaaggtccaagaccacatttacttgaaattcttcctcttttctgatttgtcttaccagatatttcccttcttgcctcattctctaggatttgaataaaacctttgccacttattttattagccttattaatgtcacctttccttcaactcaaatatttgagaaattttatgttacatatatgaatgaagttgaaagctactgtttggaagctaagacttgtatagagtagttttgtatatgaatggttgttctagctcagatttcaggttactcacaacagtattctttctaagaggatatatacaaatgtctctcataatttatgtaaggaaatattatgaaaatgagacttttttagtttgatgtgtgtttcccaaactagagataaaactagtaagatttagtatcgtttataacatttgaatttttgctccatgagtacaactaatttttataagtaaatattgggtttatatgaaaaatggggtgtcagtcttttcacacgcccagcaggtaacaatgtgggaagctggcagggtcattgatagcaagtaagtacttcctgaaggctttccagttcaaaagattacaagccattctgcctgccaaacaaattatattctgaagatgcctgttttgtaacccttgatgtgaattttttggtgtctgaaatttacaaaagaatgaaattgaaattgtaaaacactaaatgctttgggtttattttgaagtaatctgttactttaaaatgtcaacattaggaagccataaaacaagatattatgaaacccagtattataaatgttatctacatctaaagtattttaaaataacttattggcagctttattctttttttccttacaagatttagaatctttttggttatatgtctatttttcaattttgttatatttttaatttaagtggccaatgtggttatgaacaagatttgtatggtcagcttctgttctttcctaaaacttcagataaatatcattttagctataacctaaaaaagtgtttaaataaaatgacagatgttaatttaaaagcagcatatgctaatttactttttcatatgatgatggtctaatggaagttacatatgctttcttttgtcctaactctgaaaagtatatgtcagagttctggaatatgtctttagccaagaattttattcacttaaatttgtttacaacttgtataaagcaaaaaagaatgtgtgtaactatagtgaacgcatagttttgcttatattatgtgatgtttgccaaaaaaagaataaaaaatgaatgaattaaatcaacaaaaaaaaa

SEQ ID NO: 12 - DNA, 9068 bp, Homo sapiens
cggcgcgcgcgcggggcgggggcgcgcggaggggggggctgccccggggcggcccccccaggtcggggcgcggcgggcggcggcggcgggcgcgcgtcccgtccaggtccggagtaaccgccgccgccgccgccaaagctcgccaacatggcggacctggaggctgtgctggccgatgtcagttacctgatggccatggagaagagcaaggcgaccccggccgcccgcgccagcaagaggatcgtcctgccggagcccagtatccggagtgtgatgcagaagtaccttgcagagagaaatgaaataacctttgacaagattttcaatcagaaaattggtttcttgctatttaaagatttttgtttgaatgaaattaatgaagctgtacctcaggtgaagttttatgaagagataaaggaatatgaaaaacttgataatgaggaagaccgcctttgcagaagtcgacaaatttatgatgcctacatcatgaaggaacttctttcctgttcacatcctttctcaaagcaagctgtagaacacgtacaaagtcatttatccaagaaacaagtgacatcaactctttttcagccatacatagaagaaatttgtgaaagccttcgaggtgacatttttcaaaaatttatggaaagtgacaagttcactagattttgtcagtggaaaaacgttgaattaaatatccatttgaccatgaatgagttcagtgtgcataggattattggacgaggaggattcggggaagtttatggttgcaggaaagcagacactggaaaaatgtatgcaatgaaatgcttagataagaagaggatcaaaatgaaacaaggagaaacattagccttaaatgaaagaatcatgttgtctcttgtcagcacaggagactgtcctttcattgtatgtatgacctatgccttccataccccagataaactctgcttcatcctggatctgatgaacgggggcgatttgcactaccacctttcacaacacggtgtgttctctgagaaggagatgcggttttatgccactgaaatcattctgggtctggaacacatgcacaatcggtttgttgtctacagagatttgaagccagcaaatattctcttggatgaacatggacacgcaagaatatcagatcttggtcttgcctgcgatttttccaaaaagaagcctcatgcgagtgttggcacccatgggtacatggctcccgaggtgctgcagaaggggacggcctatgacagcagtgccgactggttctccctgggctgcatgcttttcaaacttctgagaggtcacagccctttcagacaacataaaaccaaagacaagcatgaaattgaccgaatgacactcaccgtgaatgtggaacttccagacaccttctctcctgaactgaagtcccttttggagggcttgcttcagcgagacgttagcaagcggctgggctgtcacggaggcggctcacaggaagtaaaagagcacagctttttcaaaggtgttgactggcagcatgtctacttacaaaagtacccaccacccttgattcctccccggggagaagtcaatgctgctgatgcctttgatattggctcatttgatgaagaggataccaaagggattaagctacttgattgcgaccaagaactctacaagaacttccctttggtcatctctgaacgctggcagcaagaagtaacggaaacagtttatgaagcagtaaatgcagacacagataaaatcgaggccaggaagagagctaaaaataagcaacttggccacgaagaagattacgctctggggaaggactgtattatgcacgggtacatgctgaaactgggaaacccatttctgactcagtggcagcgtcgctatttttacctctttccaaatagacttgaatggagaggagagggagagtcccggcaaaatttactgacaatggaacagattctctctgtggaagaaactcaaattaaagacaaaaaatgcattttgttcagaataaaaggagggaaacaatttgtcttgcaatgtgagagtgatccagagtttgtgcagtggaagaaagagttgaacgaaaccttcaaggaggcccagcggctattgcgtcgtgccccgaagttcctcaacaaacctcggtcaggtactgtggagctcccaaagccatccctctgtcacagaaacagcaacggcctctagcacccagaaacagggagggtcctcgaggaggacacaccagggtctcagccttttggggtgaacgaggatgaggcatctgatctattcgctaccgggactcctccaggctcccgagaggagtcgggacccttcggcttggggtcagctcagctccctgccttgtcacatttgtctgcattagaaactactgaagaaataaaagttctttttctttgctacacactttggtacctatgaacctagaacttgaagtgactcctacttatcacgtaaatttttatgtctgatatcaaacacatcttagactccccagaatggaatttaaagatgttcagtgttgggtaacagattgccctaagcattgccacatattctgtctagtcactgctgattttctatgtctttgctccatactgctgggggatgggagagccacagtgtgtttcttttgtgcacttcgcaactgacttcttgtcctggggttaaaagttgaagatattttctgatgatattaaaagttgaagatatttctgcacttgggccctcctctgggagccgcacccacatgactgccctgcctctgaccagtctgttccggggccccctcagccaggtgggaatgacggacacgtactatccaagtgtatgggattaactaatcattgaaggcattcatccgtccatcattggaaagatttacagtgattctgaaggacaggccgtggagttttaggtttcaggggcaagagcagttttcaaaagtctttgagtccagtgtgcacgagtcgacaagcagtacctggcatgcaggagcactcatgggtgagtccgtctcaggtctcgacaattagcagttgtgtgacagtcattctggttccttctgcctgaccctgggagacatatcagtaatggatgtacaaaagcaggtctgttttatgtcttagtataatttcagatgaattgtattgaaaaaatgctgaggaatgaatgtgtcaaaatgggttaactgtgtatattgactttcatgtcgtcatgcatctgtcatgaatgaatgatactttgcactgggctgtacgacagtgaggaccttagggcatgaagcctttttcctggtcccagcagcatctgccctgtgaagtttgttttctcccactgcctccaggccccactgatacccccaaatagatgctgggttatgagaaccagcgaaatcccccatgtcatcagtcttaaaaaaaaaattttacaaatccacgtatttgtcccattcttggagtagttttagtgtatgtctttacattaactactaacagtataaataacttgacatcgtaattgtctgcatcctgtccttgatatttttagcagttccaaatctttgtttttgtatttgtttgctgtgttcatgggcaaagtaagtactttttaatgcagttattttgagagtttggaagataattaccaaaagggtccattatttcataagagttactttgcaaaaaaaaaaatgtgggtttttttttttgtctatctcaactactagttggggtttaaattaacatacattttctactatctgttatttccagtgtgggaggagggatgtactacttacatgcattctccttatttaaaaaggaagaatagtattcaaattctgttgaaacacacacacacacacacacacacacacacacacactccagaagcagaaaagccattgttcttaaagagtgaatgtcttcccagccctggttaattatagctgtgactgatgccgttcccgtctgcatctcaagctcataggttctcagcatgtgcagttgaggatgcgctgggcctcatgcctgttctagatctccaggataaagggcctgctgttgactccaccagggtctgggcttagcgtctaatatctcgtacctagggcgtgagctgcacaaacgtgttcagaaagattattcaactttcccatacttgttctaaaattgagctgatccgcatctctttcaaaaactagaatttctgctctaagaatagaacataaggctccactcccttttagaaaagatatatgaattggaaaatgctctgaaagtccttttgcttcaaacaaaagtgtaaacttttacacttccccaactcacatttgatttgtaatgatatggttgagaagtacatctagatgtcatttattaaaagtgctttgtaagactagattgagctgtttctgagggcggtcaccagttgtgttggggtctggtttgagtgccttctgccaaaatgttgtgatggaggtgtttctgcgaccagacacaggataccgctgtgtctgcacccggttgcctgcatggccagaggaaaagtcagttggattaaacatcatggtatacttggctgttgtttttttttaattttttaattttttgggatagggcctcgctctgtcacccaggctggagaacagtgggatgatcatggctcactgcagccttgaattcctaggttcaagcaatcctcccacgtcagcctcctgagtagctaggactacaggtgcatgccacctttcctggctaatttattttttgggtagagatggggtcttgaactcttaggctcaagtgatcctccttccttggcctcccaaaatgctggaattagagatgtaagccaccatgcccagccatagtacttggatgttttagaaggttttccaagtattacataattcctagatgttcacccttattacactccaactattaaaaaggtcaaaattcagcctattttttttcattattttagattcctgtggttgggatattttaacattgatgagaaaaataattgaggttgatatttttacaaaatcatgcggtaataagtcttgatttcatgattcaaaagaatcaataaagcctaaaaataatagattactttaagctgctatgtaagatatatatggaataaattaaaaacctttgtgaattcaggtttattatttttaacctaaaacattctctttggttcattcatcccctcatgtcatgggggctcattggttttccttctttgtcatatttaagtatgatttttcaacaaaacttctagaagtcagcttattatgtcaccattcatgcaaagtgctcatgcctctgattggtccattcactgacgtgacaatttcaggtcctatgtttaaaaagaaggggctggccgggcacgatggctcacgcctataatcccagcactttgggaggccgagaggggcggttcacgaggtcaggagattgagaccatcctggttagcagagtgaaaccccgtctctactaaaaatacaaataaaaattagccgggcgtggtggcgggcgcctgtagtcccagctacttgggaggctgaggcaggagaatggcatgaacccgggaggcagagcttgcagtgagccgagattgcgccactgcactccagcctgggcgacagagcgagactctgtctcaaaaaaaaaaaggaggggggctaaatatccagtgagatgcactgaggaaaggaagcattttgctgaagacagcagcagcaacaaacaatggtctgtttgttgcaaacaagatgtagcttgatttctggtctgacatatgccatatacagatattagaaacgactgtttgaaggccacactggtcatctacaaagtaatgtttaccaattgacgacagggatttaactagattaaaaagatcaaagtgtggtttttctctgctttttaaaatttcactcggaatttgtagctgggccaattcaacacattttacttttcagtggaattgatttttctaatgtttcagaattttaacatatcaagaagaaaacaacgttctcaaagtctggcctctttagcatgatgtaaacctatagaaatgctttgaaatgtgctggtgtaagataagagttatcttgtatgatttaatcatatgcagtgttgtctcagttacgttcagggaaatgtttctgtgtcattcagagatgcttgatgaattaacacctcccaccctgagtgaggggttgacttgttgggagatgatttgggcttcactgggatctgtgacaggtgggggctgggctgggtgtcacaaagagaatagtggtagaaatcgggcgaaggaagaaagaagttactggtaaaaatcattacaccataaagcaccaaggaaataactgagttaaaataggtgaagtttcttttttcccccctgtaacaggagagttttccttatgataattattctgagacttggtcactttgtttttgaatgtggagctgctgaactcattcagaagccatttgctgcctatcaggactttctgaagaagttcttttgcctctgcctaccctctggcaccctcccatggaggcacaggggacccagagctaaagcattaccaggccatctccaaaacaccccgtgtgtgtgtgtgtgtgtgtgtgtgtgtgtgtgtgtgtgtgtgtgcactttgcagcccccgaggtggagaggcagtgtctggatcactgtgaatgcattgccccattggtcagttggggacactgttacaaatccactgaagtcctggtaaaactgtcaagagtaacaggcctcttctgttctaccctgctcacttccacggtgagttaccagcctgggcaacacagcaagaccccatctctacaaaaaaaatttttttaagtaattaaccgtttaaattttttcctaaagatttaacatgatttttccctcctatgtaaagtttactggagagacttgaattacttaaattcatgttaatatgatttttttttaatccaggtcacattttaacaaagtttattatgaaacaaatgaaatttgaactctaaaatggtactccttggcttcctcaagtcacaatgaactttatattttctttgtccttaaggactaagatagttgttttatttcagccgaatcacagagataaccactcctgcaggcccccacagctggcccaaaggggctgtctttctgacctggctgtgttagcactgattgagaaacgcaggctcccaaattttaaattgcctttattaaaaacacaaactacagaaaatgggttaagagtatacgcatttcatcaaacacatataggggaaaaaatccttcaatttagagttaaataactcagctttgtatagtagagttagcgctccagtatctaacaatctcagaatcatctctgaaaactggtaactatgcttccatttttaattttgtcctaaatatcagatgtctttgatgtaagggtagggaatggagaaatattttcaattgtgtatttgtattacaaagaacttgaaatttactttcttagttgattatattaaatgatgtatatattatatgtggtttataagctcaacactggccatttttttagttttattgttaaatggtatttttctatgtttaattataatagatctggctttttctggatagcataaagatcactgaactatatatatataagaaacaagagttctattttagcacaaaggcattttatattatttattgaatccataagtttgttttcgtcaaaaacattccatattatttctgctcctttttatttgtatagtttgttatttaaagaaatggcagtccttcctgttcttaatacaataaaattgaaataatgcacctagtaatgtggccgacatctcttctcaccaccatggactgttttcaacaacagttgatcttctggtctgtgctgagaggcgcatgcatgtctttcgtcacgtcgggcagcacacctgctgtgaaatactgctttcatctacctcttcagaaggcttcttgcttgttgacaagtaccgcaaaggctttattctggactggctatctcataaaaggatttctgtaagactttgcagtgtcattccctcagaacctaggtttgtttctaaagccacggtattgtccaggagcccctgtgtgtggggcaggtagctatccctcccatgtcattagtaatcctttaggatttaaggtacaactggacagcatcattccttccccttattgtgccaaatccccaccatcagccttgccattgccttaagatttgattattgcacccaattacctaaccactaaacagaaaggccaccttcactctttgaaaaaggcaagctgtgcttagaaacactgcttttaagagtagcacatttgagtgtgactttttccccccttcactatttcaaaatggttttgaaatggggtcttaaaggtaagcgccctcatacatgactgaaactttgtgagaggtcttatatttgaatggacccttaatgatttatgtgaaatagaatgaagtcctgtctctgtgagagaacgtgcctcctcactcatttgtctctgtctgttttcatagccatcaatatagtaacatatttactatattcttgaatacccttgaagaaagaaatccgttttctattgtgcattgctatacgaagtgaagccagtaaactagatactgtaaatctagatattgtacctagacaaaatatcattggttctatctctttttgtatctgttgtgccagggaaggtttataatcccttctcagtatacactcactagtgcacgtctgaaatagtatcccacgggagatgctgctccacgtctgaggtcacctgccctgtgtggggcacaccaccgtcagcaccaccgtttttacagttactttggagctgctagactggttttctgtgttggtaaattgcctatataaatctgaataaaaaggatctgtacaaaaaaaaaaaaaaa

SEQ ID NO: 13 - DNA, 2096 bp, Homo sapiens
gggcctgtggctggccgggggcggagaagcggggggtcggggtccctccccctggcgctggctcaggaatccgccgaagggcgggcggaggcgccggggtgggccgcgccgcggcaggcgggcgggcggggggcgcttcctggggccgcgcgtccagggagctgtgccgtccgcccgtccgtctgcccgcaggcattgcccgagccagccgagccgccagagccgcgggccgcgggggtgtcgcgggcccaaccccaggatgctcccctgcgcctcctgcctacccgggtctctactgctctgggcgctgctactgttgctcttgggatcagcttctcctcaggattctgaagagcccgacagctacacggaatgcacagatggctatgagtgggacccagacagccagcactgccgggatgtcaacgagtgtctgaccatccctgaggcctgcaagggggaaatgaagtgcatcaaccactacgggggctacttgtgcctgccccgctccgctgccgtcatcaacgacctacacggcgagggacccccgccaccagtgcctcccgctcaacaccccaacccctgcccaccaggctatgagcccgacgatcaggacagctgtgtggatgtggacgagtgtgcccaggccctgcacgactgtcgccccagccaggactgccataacttgcctggctcctatcagtgcacctgccctgatggttaccgcaagatcgggcccgagtgtgtggacatagacgagtgccgctaccgctactgccagcaccgctgcgtgaacctgcctggctccttccgctgccagtgcgagccgggcttccagctggggcctaacaaccgctcctgtgttgatgtgaacgagtgtgacatgggggccccatgcgagcagcgctgcttcaactcctatgggaccttcctgtgtcgctgccaccagggctatgagctgcatcgggatggcttctcctgcagtgatattgatgagtgtagctactccagctacctctgtcagtaccgctgcatcaacgagccaggccgtttctcctgccactgcccacagggttaccagctgctggccacacgcctctgccaagacattgatgagtgtgagtctggtgcgcaccagtgctccgaggcccaaacctgtgtcaacttccatgggggctaccgctgcgtggacaccaaccgctgcgtggagccctacatccaggtctctgagaaccgctgtctctgcccggcctccaaccctctatgtcgagagcagccttcatccattgtgcaccgctacatgaccatcacctcggagcggagcgtgcccgctgacgtgttccagatccaggcgacctccgtctaccccggtgcctacaatgcctttcagatccgtgctggaaactcgcagggggacttttacattaggcaaatcaacaacgtcagcgccatgctggtcctcgcccggccggtgacgggcccccgggagtacgtgctggacctggagatggtcaccatgaattccctcatgagctaccgggccagctctgtactgaggctcaccgtctttgtaggggcctacaccttctgaggagcaggagggagccaccctccctgcagctaccctagctgaggagcctgttgtgaggggcagaatgagaaaggcaataaagggagaaagaaagtcctggtggctgaggtgggcgggtcacactgcaggaagcctcaggctggggcagggtggcacttgggggggcaggccaagttcacctaaatgggggtctctatatgttcaggcccaggggcccccattgacaggagctgggagctctgcaccacgagcttcagtcaccccgagaggagaggaggtaacgaggagggcggactccaggccccggcccagagatttggacttggctggcttgcaggggtcctaagaaactccactctggacagcgccaggaggccctgggttccattcctaactctgcctcaaactgtacatttggataagccctagtagttccctgggcctgtttttctataaaacgaggcaactggactgttaaaaaaaaaaaaaaaaaaaaaaaaaaaaa

SEQ ID NO: 14 - DNA, 3847 bp, Homo sapiens
agagactctcactgcacgccggagggcgcccttcctcgctcgcgcccgcgcgaccgcgcgccccagtcccgccccgccccgctaaccgccccagacacagcgctcgccgagggtcgcttggaccctgatcttacccgtgggcaccctgcgctctgcctgccgcgaagaccggctccccgacccgcagaagtcaggagagagggtgaagcggagcagcccgaggcggggcagcctcccggagcagcgccgcgcagagcccgggacaatggggccgcggcggctgctgctggtggccgcctgcttcagtctgtgcggcccgctgttgtctgcccgcacccgggcccgcaggccagaatcaaaagcaacaaatgccaccttagatccccggtcatttcttctcaggaaccccaatgataaatatgaaccattttgggaggatgaggagaaaaatgaaagtgggttaactgaatacagattagtctccatcaataaaagcagtcctcttcaaaaacaacttcctgcattcatctcagaagatgcctccggatatttgaccagctcctggctgacactctttgtcccatctgtgtacaccggagtgtttgtagtcagcctcccactaaacatcatggccatcgttgtgttcatcctgaaaatgaaggtcaagaagccggcggtggtgtacatgctgcacctggccacggcagatgtgctgtttgtgtctgtgctcccctttaagatcagctattacttttccggcagtgattggcagtttgggtctgaattgtgtcgcttcgtcactgcagcattttactgtaacatgtacgcctctatcttgctcatgacagtcataagcattgaccggtttctggctgtggtgtatcccatgcagtccctctcctggcgtactctgggaagggcttccttcacttgtctggccatctgggctttggccatcgcaggggtagtgcctctgctcctcaaggagcaaaccatccaggtgcccgggctcaacatcactacctgtcatgatgtgctcaatgaaaccctgctcgaaggctactatgcctactacttctcagccttctctgctgtcttcttttttgtgccgctgatcatttccacggtctgttatgtgtctatcattcgatgtcttagctcttccgcagttgccaaccgcagcaagaagtcccgggctttgttcctgtcagctgctgttttctgcatcttcatcatttgcttcggacccacaaacgtcctcctgattgcgcattactcattcctttctcacacttccaccacagaggctgcctactttgcctacctcctctgtgtctgtgtcagcagcataagctgctgcatcgaccccctaatttactattacgcttcctctgagtgccagaggtacgtctacagtatcttatgctgcaaagaaagttccgatcccagcagttataacagcagtgggcagttgatggcaagtaaaatggatacctgctctagtaacctgaataacagcatatacaaaaagctgttaacttaggaaaagggactgctgggaggttaaaaagaaaagtttataaaagtgaataacctgaggattctattagtccccacccaaactttattgattcacctcctaaaacaacagatgtacgacttgcatacctgctttttatgggagctgtcaagcatgtatttttgtcaattaccagaaagataacaggacgagatgacggtgttattccaagggaatattgccaatgctacagtaataaatgaatgtcacttctggatatagctaggtgacatatacatacttacatgtgtgtatatgtagatgtatgcacacacatatattatttgcagtgcagtatagaataggcactttaaaacactctttccccgcaccccagcaattatgaaaataatctctgattccctgatttaatatgcaaagtctaggttggtagagtttagccctgaacatttcatggtgttcatcaacagtgagagactccatagtttgggcttgtaccacttttgcaaataagtgtattttgaaattgtttgacggcaaggtttaagttattaagaggtaagacttagtactatctgtgcgtagaagttctagtgttttcaattttaaacatatccaagtttgaattcctaaaattatggaaacagatgaaaagcctctgttttgatatgggtagtattttttacattttacacactgtacacataagccaaaactgagcataagtcctctagtgaatgtaggctggctttcagagtaggctattcctgagagctgcatgtgtccgcccccgatggaggactccaggcagcagacacatgccagggccatgtcagacacagattggccagaaaccttcctgctgagcctcacagcagtgagactggggccactacatttgctccatcctcctgggattggctgtgaactgatcatgtttatgagaaactggcaaagcagaatgtgatatcctaggaggtaatgaccatgaaagacttctctacccatcttaaaaacaacgaaagaaggcatggacttctggatgcccatccactgggtgtaaacacatctagtagttgttctgaaatgtcagttctgatatggaagcacccattatgcgctgtggccactccaataggtgctgagtgtacagagtggaataagacagagacctgccctcaagagcaaagtagatcatgcatagagtgtgatgtatgtgtaataaatatgtttcacacaaacaaggcctgtcagctaaagaagtttgaacatttgggttactatttcttgtggttataacttaatgaaaacaatgcagtacaggacatatattttttaaaataagtctgatttaattgggcactatttatttacaaatgttttgctcaatagattgctcaaatcaggttttcttttaagaatcaatcatgtcagtctgcttagaaataacagaagaaaatagaattgacattgaaatctaggaaaattattctataatttccatttacttaagacttaatgagactttaaaagcattttttaacctcctaagtatcaagtatagaaaatcttcatggaattcacaaagtaatttggaaattaggttgaaacatatctcttatcttacgaaaaaatggtagcattttaaacaaaatagaaagttgcaaggcaaatgtttatttaaaagagcaggccaggcgcggtggctcacgcctgtaatcccagcactttgggaggctgaggcgggtggatcacgaggtcaggagatcgagaccatcctggctaacacggtgaaacccgtctctactaaaaatgcaaaaaaaattagccgggcgtggtggcaggcacctgtagtcccagctactcgggaggctgaggcaggagactggcgtgaacccaggaggcggaccttgtagtgagccgagatcgcgccactgtgctccagcctgggcaacagagcaagactccatctcaaaaaataaaaataaataaaaaataaaaaaataaaagagcaaactatttccaaataccatagaataacttacataaaagtaatataactgtattgtaagtagaagctagcactggttttattaatttagtgactattcattttatctaaatcagtgaagatttactgtcattgtttattagtctgtatatattaaaatatgatatcattaatgtacttacaaaatagtatgtcactgtttttatgttcattcttaaaaacataacctgtattaataaatgtgaacatttgcttggtaaaaaaaaaaaaaaaaaaaaaaaaaaaaaaaaaaaaaa

SEQ ID NO: 15 - DNA, 2134 bp, Homo sapiens
ggagttgagaattagggaggaggtggtagagtccgggtagtgagcggagggacaggaagggtagggcaagaaagggagaggggacaggagggaagggtgggccaaagcggtgagaaaggagggccagccagttgggtgggggagagggccgaggcccgggggcaggagtgcagggctctgaggcggggagaggagaggagagaagagccgcggggggcccagcccggagccaggatgcccgcgccgcgcgcccgggagcagccccgcgtgcccggggagcgccagccgctgctgcctcgcggtgcgcggggccctcgacggtggcggcgggcggcgggcgcggccgtgctgctggtggagatgctggagcgcgccgccttcttcggcgtcaccgccaacctcgtgctgtacctcaacagcaccaacttcaactggaccggcgagcaggcgacgcgcgccgcgctggtattcctgggcgcctcctacctgctggcgcccgtgggcggctggctggccgacgtgtacctgggccgctaccgcgcggtcgcgctcagcctgctgctctacctggccgcctcgggcctgctgcccgccaccgccttccccgacggccgcagctccttctgcggagagatgcccgcgtcgccgctgggacctgcctgcccctcggccggctgcccgcgctcctcgcccagcccctactgcgcgcccgtcctctacgcgggcctgctgctactcggcctggccgccagctccgtccggagcaacctcacctccttcggtgccgaccaggtgatggatctcggccgcgacgccacccgccgcttcttcaactggttttactggagcatcaacctgggtgctgtgctgtcgctgctggtggtggcgtttattcagcagaacatcagcttcctgctgggctacagcatccctgtgggctgtgtgggcctggcatttttcatcttcctctttgccacccccgtcttcatcaccaagcccccgatgggcagccaagtgtcctctatgcttaagctcgctctccaaaactgctgcccccagctgtggcaacgacactcggccagagaccgtcaatgtgcccgcgtgctggccgacgagaggtctccccagccaggggcttccccgcaagaggacatcgccaacttccaggtgctggtgaagatcttgcccgtcatggtgaccctggtgccctactggatggtctacttccagatgcagtccacctatgtcctgcagggtcttcacctccacatcccaaacattttcccagccaacccggccaacatctctgtggccctgagagcccagggcagcagctacacgatcccggaagcctggctcctcctggccaatgttgtggtggtgctgattctggtccctctgaaggaccgcttgatcgaccctttactgctgcggtgcaagctgcttccctctgctctgcagaagatggcgctggggatgttctttggttttacctccgtcattgtggcaggagtcctggagatggagcgcttacactacatccaccacaacgagaccgtgtcccagcagattggggaggtcctgtacaacgcggcaccactgtccatctggtggcagatccctcagtacctgctcattgggatcagtgagatctttgccagcatcccaggcctggagtttgcctactcagaggccccgcgctccatgcagggcgccatcatgggcatcttcttctgcctgtcgggggtgggctcactgttgggctccagcctagtggcactgctgtccttgcccgggggctggctgcactgccccaaggactttgggaacatcaacaattgccggatggacctctacttcttcctgctggctggcattcaggccgtcacggctctcctatttgtctggatcgctggacgctatgagagggcgtcccagggcccagcctcccacagccgtttcagcagggacaggggctgaacaggccctattccagcccccttgcttcactctaccggacagacggcagcagtcccagctctggtttccttctcggtttattctgttagaatgaaatggttcccataaataaggggcatgagcccttcctcacgacaaaaaaaaaaaaaaaaaa

SEQ ID NO: 16 - DNA, 6194 bp, Homo sapiens
actaactcgcggctgcaggatcagcgtctggaagcagacgtttcggctacagacccagagaggaggagctggagatcaggaggcgtgagccgccaagagtttgcagaatctgtggtgtgaatgaactgggggcacctgggcgcacagatcgccccccttcccccgccccgggccacagttgagtagtggtacatttttttcaccctcttgtgaagaatttctttttattattatttgtcgtaaggtcttttgcacaatcacgcccacatttggggttggaaagccctaattaccgccgtcgctgatggacgttggaaacggagcgcctctccgtggaacagttgcctgcgcgccctcgccggaccggcggctccctagttgcgccccgaccaggccctgcccttgctgccggctcgcgcgcgtccgcgccccctccattcctgggcgcatcccagctctgccccaactcgggagtccaggcccgggcgccagtgcccgcttcagctccggttcactgcgcccgccggacgcgcgccggaggactccgcagccctgctcctgaccgtccccccaggcttaacccggtcgctccgctcggattcctcggctgcgctcgctcgggtggcgacttcctccccgcgccccctccccctcgccatgaagaagtccattggaatattaagcccaggagttgctttggggatggctggaagtgcaatgtcttccaagttcttcctagtggctttggccatatttttctccttcgcccaggttgtaattgaagccaattcttggtggtcgctaggtatgaataaccctgttcagatgtcagaagtatatattataggagcacagcctctctgcagccaactggcaggactttctcaaggacagaagaaactgtgccacttgtatcaggaccacatgcagtacatcggagaaggcgcgaagacaggcatcaaagaatgccagtatcaattccgacatcgaaggtggaactgcagcactgtggataacacctctgtttttggcagggtgatgcagataggcagccgcgagacggccttcacatacgcggtgagcgcagcaggggtggtgaacgccatgagccgggcgtgccgcgagggcgagctgtccacctgcggctgcagccgcgccgcgcgccccaaggacctgccgcgggactggctctggggcggctgcggcgacaacatcgactatggctaccgctttgccaaggagttcgtggacgcccgcgagcgggagcgcatccacgccaagggctcctacgagagtgctcgcatcctcatgaacctgcacaacaacgaggccggccgcaggacggtgtacaacctggctgatgtggcctgcaagtgccatggggtgtccggctcatgtagcctgaagacatgctggctgcagctggcagacttccgcaaggtgggtgatgccctgaaggagaagtacgacagcgcggcggccatgcggctcaacagccggggcaagttggtacaggtcaacagccgcttcaactcgcccaccacacaagacctggtctacatcgaccccagccctgactactgcgtgcgcaatgagagcaccggctcgctgggcacgcagggccgcctgtgcaacaagacgtcggagggcatggatggctgcgagctcatgtgctgcggccgtggctacgaccagttcaagaccgtgcagacggagcgctgccactgcaagttccactggtgctgctacgtcaagtgcaagaagtgcacggagatcgtggaccagtttgtgtgcaagtagtgggtgccacccagcactcagccccgctcccaggacccgcttatttatagaaagtacagtgattctggtttttggtttttagaaatattttttatttttccccaagaattgcaaccggaaccattttttttcctgttaccatctaagaactctgtggtttattattaatattataattattatttggcaataatgggggtgggaaccaagaaaaatatttattttgtggatctttgaaaaggtaatacaagacttcttttgatagtatagaatgaaggggaaataacacataccctaacttagctgtgtggacatggtacacatccagaaggtaaagaaatacattttctttttctcaaatatgccatcatatgggatgggtaggttccagttgaaagagggtggtagaaatctattcacaattcagcttctatgaccaaaatgagttgtaaattctctggtgcaagataaaaggtcttgggaaaacaaaacaaaacaaaacaaacctcccttccccagcagggctgctagcttgctttctgcattttcaaaatgataatttacaatggaaggacaagaatgtcatattctcaaggaaaaaaggtatatcacatgtctcattctcctcaaatattccatttgcagacagaccgtcatattctaatagctcatgaaatttgggcagcagggaggaaagtccccagaaattaaaaaatttaaaactcttatgtcaagatgttgatttgaagctgttataagaattaggattccagattgtaaaaagatccccaaatgattctggacactagatttttttgtttggggaggttggcttgaacataaatgaaaatatcctgttattttcttagggatacttggttagtaaattataatagtaaaaataatacatgaatcccattcacaggttctcagcccaagcaacaaggtaattgcgtgccattcagcactgcaccagagcagacaacctatttgaggaaaaacagtgaaatccaccttcctcttcacactgagccctctctgattcctccgtgttgtgatgtgatgctggccacgtttccaaacggcagctccactgggtcccctttggttgtaggacaggaaatgaaacattaggagctctgcttggaaaacagttcactacttagggatttttgtttcctaaaacttttattttgaggagcagtagttttctatgttttaatgacagaacttggctaatggaattcacagaggtgttgcagcgtatcactgttatgatcctgtgtttagattatccactcatgcttctcctattgtactgcaggtgtaccttaaaactgttcccagtgtacttgaacagttgcatttataaggggggaaatgtggtttaatggtgcctgatatctcaaagtcttttgtacataacatatatatatatatacatatatataaatataaatataaatatatctcattgcagccagtgatttagatttacagtttactctggggttatttctctgtctagagcattgttgtccttcactgcagtccagttgggattattccaaaagttttttgagtcttgagcttgggctgtggccctgctgtgatcataccttgagcacgacgaagcaaccttgtttctgaggaagcttgagttctgactcactgaaatgcgtgttgggttgaagatatcttttttcttttctgcctcacccctttgtctccaacctccatttctgttcactttgtggagagggcattacttgttcgttatagacatggacgttaagagatattcaaaactcagaagcatcagcaatgtttctcttttcttagttcattctgcagaatggaaacccatgcctattagaaatgacagtacttattaattgagtccctaaggaatattcagcccactacatagatagcttttttttttttttttttaataaggacacctctttccaaacagtgccatcaaatatgttcttatctcagacttacgttgttttaaaagtttggaaagatacacatctttcataccccccttaggcaggttggctttcatatcacctcagccaactgtggctcttaatttattgcataatgatattcacatcccctcagttgcagtgaattgtgagcaaaagatcttgaaagcaaaaagcactaattagtttaaaatgtcacttttttggtttttattatacaaaaaccatgaagtactttttttatttgctaaatcagattgttcctttttagtgactcatgtttatgaagagagttgagtttaacaatcctagcttttaaaagaaactatttaatgtaaaatattctacatgtcattcagatattatgtatatcttctagcctttattctgtacttttaatgtacatatttctgtcttgcgtgatttgtatatttcactggtttaaaaaacaaacatcgaaaggcttatgccaaatggaagatagaatataaaataaaacgttacttgtatattggtaagtggtttcaattgtccttcagataattcatgtggagatttttggagaaaccatgacggatagtttaggatgactacatgtcaaagtaataaaagagtggtgaattttaccaaaaccaagctatttggaagcttcaaaaggtttctatatgtaatggaacaaaaggggaattctcttttcctatatatgttccttacaaaaaaaaaaaaaaaagaaatcaagcagatggcttaaagctggttataggattgctcacattcttttagcattatgcatgtaacttaattgttttagagcgtgttgctgttgtaacatcccagagaagaatgaaaaggcacatgcttttatccgtgaccagatttttagtccaaaaaaatgtatttttttgtgtgtttaccactgcaactattgcacctctctatttgaatttactgtggaccatgtgtggtgtctctatgccctttgaaagcagtttttataaaaagaaagcccgggtctgcagagaatgaaaactggttggaaactaaaggttcattgtgttaagtgcaattaatacaagttattgtgcttttcaaaaatgtacacggaaatctggacagtgctccacagattgatacattagcctttgctttttctctttccggataaccttgtaacatattgaaaccttttaaggatgccaagaatgcattattccacaaaaaaacagcagaccaacatatagagtgtttaaaatagcatttctgggcaaattcaaactcttgtggttctaggactcacatctgtttcagtttttcctcagttgtatattgaccagtgttctttattgcaaaaacatatacccgatttagcagtgtcagcgtattttttcttctcatcctggagcgtattcaagatcttcccaatacaagaaaattaataaaaaatttatatataggcagcagcaaaagagccatgttcaaaatagtcattatgggctcaaatagaaagaagacttttaagttttaatccagtttatctgttgagttctgtgagctactgacctcctgagactggcactgtgtaagttttagttgcctaccctagctcttttctcgtacaattttgccaataccaagtttcaatttgtttttacaaaacattattcaagccactagaattatcaaatatgacgctatagcagagtaaatactctgaataagagaccggtactagctaactccaagagatcgttagcagcatcagtccacaaacacttagtggcccacaatatatagagagatagaaaaggtagttataacttgaagcatgtatttaatgcaaataggcacgaaggcacaggtctaaaatactacattgtcactgtaagctatacttttaaaatatttattttttttaaagtattttctagtcttttctctctctgtggaatggtgaaagagagatgccgtgttttgaaagtaagatgatgaaatgaatttttaattcaagaaacattcagaaacataggaattaaaacttagagaaatgatctaatttccctgttcacacaaactttacactttaatctgatgattggatattttattttagtgaaacatcatcttgttagctaactttaaaaaatggatgtagaatgattaaaggttggtatgatttttttttaatgtatcagtttgaacctagaatattgaattaaaatgctgtctcagtattttaaaagcaaaaaaggaatggaggaaaattgcatcttagaccatttttatatgcagtgtacaatttgctgggctagaaatgagataaagattatttatttttgttcatatcttgtacttttctattaaaatcattttatgaaatccaaaaaaaaaaaaaaaaaa

SEQ ID NO: 17 - DNA, 5599 bp, Homo sapiens
gctccactcgcctccgtgctcctctcgcccatggaattaattctggctccacttgttgctcggcccagaagtccattggaatattaagcccaggagttgctttggggatggctggaagtgcaatgtcttccaagttcttcctagtggctttggccatatttttctccttcgcccaggttgtaattgaagccaattcttggtggtcgctaggtatgaataaccctgttcagatgtcagaagtatatattataggagcacagcctctctgcagccaactggcaggactttctcaaggacagaagaaactgtgccacttgtatcaggaccacatgcagtacatcggagaaggcgcgaagacaggcatcaaagaatgccagtatcaattccgacatcgaaggtggaactgcagcactgtggataacacctctgtttttggcagggtgatgcagataggcagccgcgagacggccttcacatacgcggtgagcgcagcaggggtggtgaacgccatgagccgggcgtgccgcgagggcgagctgtccacctgcggctgcagccgcgccgcgcgccccaaggacctgccgcgggactggctctggggcggctgcggcgacaacatcgactatggctaccgctttgccaaggagttcgtggacgcccgcgagcgggagcgcatccacgccaagggctcctacgagagtgctcgcatcctcatgaacctgcacaacaacgaggccggccgcaggacggtgtacaacctggctgatgtggcctgcaagtgccatggggtgtccggctcatgtagcctgaagacatgctggctgcagctggcagacttccgcaaggtgggtgatgccctgaaggagaagtacgacagcgcggcggccatgcggctcaacagccggggcaagttggtacaggtcaacagccgcttcaactcgcccaccacacaagacctggtctacatcgaccccagccctgactactgcgtgcgcaatgagagcaccggctcgctgggcacgcagggccgcctgtgcaacaagacgtcggagggcatggatggctgcgagctcatgtgctgcggccgtggctacgaccagttcaagaccgtgcagacggagcgctgccactgcaagttccactggtgctgctacgtcaagtgcaagaagtgcacggagatcgtggaccagtttgtgtgcaagtagtgggtgccacccagcactcagccccgctcccaggacccgcttatttatagaaagtacagtgattctggtttttggtttttagaaatattttttatttttccccaagaattgcaaccggaaccattttttttcctgttaccatctaagaactctgtggtttattattaatattataattattatttggcaataatgggggtgggaaccaagaaaaatatttattttgtggatctttgaaaaggtaatacaagacttcttttgatagtatagaatgaaggggaaataacacataccctaacttagctgtgtggacatggtacacatccagaaggtaaagaaatacattttctttttctcaaatatgccatcatatgggatgggtaggttccagttgaaagagggtggtagaaatctattcacaattcagcttctatgaccaaaatgagttgtaaattctctggtgcaagataaaaggtcttgggaaaacaaaacaaaacaaaacaaacctcccttccccagcagggctgctagcttgctttctgcattttcaaaatgataatttacaatggaaggacaagaatgtcatattctcaaggaaaaaaggtatatcacatgtctcattctcctcaaatattccatttgcagacagaccgtcatattctaatagctcatgaaatttgggcagcagggaggaaagtccccagaaattaaaaaatttaaaactcttatgtcaagatgttgatttgaagctgttataagaattaggattccagattgtaaaaagatccccaaatgattctggacactagatttttttgtttggggaggttggcttgaacataaatgaaaatatcctgttattttcttagggatacttggttagtaaattataatagtaaaaataatacatgaatcccattcacaggttctcagcccaagcaacaaggtaattgcgtgccattcagcactgcaccagagcagacaacctatttgaggaaaaacagtgaaatccaccttcctcttcacactgagccctctctgattcctccgtgttgtgatgtgatgctggccacgtttccaaacggcagctccactgggtcccctttggttgtaggacaggaaatgaaacattaggagctctgcttggaaaacagttcactacttagggatttttgtttcctaaaacttttattttgaggagcagtagttttctatgttttaatgacagaacttggctaatggaattcacagaggtgttgcagcgtatcactgttatgatcctgtgtttagattatccactcatgcttctcctattgtactgcaggtgtaccttaaaactgttcccagtgtacttgaacagttgcatttataaggggggaaatgtggtttaatggtgcctgatatctcaaagtcttttgtacataacatatatatatatatacatatatataaatataaatataaatatatctcattgcagccagtgatttagatttacagtttactctggggttatttctctgtctagagcattgttgtccttcactgcagtccagttgggattattccaaaagttttttgagtcttgagcttgggctgtggccctgctgtgatcataccttgagcacgacgaagcaaccttgtttctgaggaagcttgagttctgactcactgaaatgcgtgttgggttgaagatatcttttttcttttctgcctcacccctttgtctccaacctccatttctgttcactttgtggagagggcattacttgttcgttatagacatggacgttaagagatattcaaaactcagaagcatcagcaatgtttctcttttcttagttcattctgcagaatggaaacccatgcctattagaaatgacagtacttattaattgagtccctaaggaatattcagcccactacatagatagcttttttttttttttttttaataaggacacctctttccaaacagtgccatcaaatatgttcttatctcagacttacgttgttttaaaagtttggaaagatacacatctttcataccccccttaggcaggttggctttcatatcacctcagccaactgtggctcttaatttattgcataatgatattcacatcccctcagttgcagtgaattgtgagcaaaagatcttgaaagcaaaaagcactaattagtttaaaatgtcacttttttggtttttattatacaaaaaccatgaagtactttttttatttgctaaatcagattgttcctttttagtgactcatgtttatgaagagagttgagtttaacaatcctagcttttaaaagaaactatttaatgtaaaatattctacatgtcattcagatattatgtatatcttctagcctttattctgtacttttaatgtacatatttctgtcttgcgtgatttgtatatttcactggtttaaaaaacaaacatcgaaaggcttatgccaaatggaagatagaatataaaataaaacgttacttgtatattggtaagtggtttcaattgtccttcagataattcatgtggagatttttggagaaaccatgacggatagtttaggatgactacatgtcaaagtaataaaagagtggtgaattttaccaaaaccaagctatttggaagcttcaaaaggtttctatatgtaatggaacaaaaggggaattctcttttcctatatatgttccttacaaaaaaaaaaaaaaaagaaatcaagcagatggcttaaagctggttataggattgctcacattcttttagcattatgcatgtaacttaattgttttagagcgtgttgctgttgtaacatcccagagaagaatgaaaaggcacatgcttttatccgtgaccagatttttagtccaaaaaaatgtatttttttgtgtgtttaccactgcaactattgcacctctctatttgaatttactgtggaccatgtgtggtgtctctatgccctttgaaagcagtttttataaaaagaaagcccgggtctgcagagaatgaaaactggttggaaactaaaggttcattgtgttaagtgcaattaatacaagttattgtgcttttcaaaaatgtacacggaaatctggacagtgctccacagattgatacattagcctttgctttttctctttccggataaccttgtaacatattgaaaccttttaaggatgccaagaatgcattattccacaaaaaaacagcagaccaacatatagagtgtttaaaatagcatttctgggcaaattcaaactcttgtggttctaggactcacatctgtttcagtttttcctcagttgtatattgaccagtgttctttattgcaaaaacatatacccgatttagcagtgtcagcgtattttttcttctcatcctggagcgtattcaagatcttcccaatacaagaaaattaataaaaaatttatatataggcagcagcaaaagagccatgttcaaaatagtcattatgggctcaaatagaaagaagacttttaagttttaatccagtttatctgttgagttctgtgagctactgacctcctgagactggcactgtgtaagttttagttgcctaccctagctcttttctcgtacaattttgccaataccaagtttcaatttgtttttacaaaacattattcaagccactagaattatcaaatatgacgctatagcagagtaaatactctgaataagagaccggtactagctaactccaagagatcgttagcagcatcagtccacaaacacttagtggcccacaatatatagagagatagaaaaggtagttataacttgaagcatgtatttaatgcaaataggcacgaaggcacaggtctaaaatactacattgtcactgtaagctatacttttaaaatatttattttttttaaagtattttctagtcttttctctctctgtggaatggtgaaagagagatgccgtgttttgaaagtaagatgatgaaatgaatttttaattcaagaaacattcagaaacataggaattaaaacttagagaaatgatctaatttccctgttcacacaaactttacactttaatctgatgattggatattttattttagtgaaacatcatcttgttagctaactttaaaaaatggatgtagaatgattaaaggttggtatgatttttttttaatgtatcagtttgaacctagaatattgaattaaaatgctgtctcagtattttaaaagcaaaaaaggaatggaggaaaattgcatcttagaccatttttatatgcagtgtacaatttgctgggctagaaatgagataaagattatttatttttgttcatatcttgtacttttctattaaaatcattttatgaaatccaaaaaaaaaaaaaaaaaa

SEQ ID NO: 18 - protein, 498 aa, Homo sapiens
MPHPRRYHSSERGSRGSYREHYRSRKHKRRRSRSWSSSSDRTRRRRREDSYHVRSRSSYDDRSSDRRVYDRRYCGSYRRNDYSRDRGDAYYDTDYRHSYEYQRENSSYRSQRSSRRKHRRRRRRSRTFSRSSSHSSRRAKSVEDDAEGHLIYHVGDWLQERYEIVSTLGEGTFGRVVQCVDHRRGGARVALKIIKNVEKYKEAARLEINVLEKINEKDPDNKNLCVQMFDWFDYHGHMCISFELLGLSTFDFLKDNNYLPYPIHQVRHMAFQLCQAVKFLHDNKLTHTDLKPENILFVNSDYELTYNLEKKRDERSVKSTAVRVVDFGSATFDHEHHSTIVSTRHYRAPEVILELGWSQPCDVWSIGCIIFEYYVGFTLFQTHDNREHLAMMERILGPIPSRMIRKTRKQKYFYRGRLDWDENTSAGRYVRENCKPLRRYLTSEAEEHHQLFDLIESMLEYEPAKRLTLGEALQHPFFARLRAEPPNKLWDSSRDISR

SEQ ID NO: 19 - protein, 350 aa, Homo sapiens
MPGPAAGSRARVYAEVNSLRSREYWDYEAHVPSWGNQDDYQLVRKLGRGKYSEVFEAINITNNERVVVKILKPVKKKKIKREVKILENLRGGTNIIKLIDTVKDPVSKTPALVFEYINNTDFKQLYQILTDFDIRFYMYELLKALDYCHSKGIMHRDVKPHNVMIDHQQKKLRLIDWGLAEFYHPAQEYNVRVASRYFKGPELLVDYQMYDYSLDMWSLGCMLASMIFRREPFFHGQDNYDQLVRIAKVLGTEELYGYLKKYHIDLDPHFNDILGQHSRKRWENFIHSENRHLVSPEALDLLDKLLRYDHQQRLTAKEAMEHPYFYPVVKEQSQPCADNAVLSSGLTAAR

SEQ ID NO: 20 - protein, 1014 aa, Homo sapiens
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNEYIVYDIAQVNLKYLLKLKFNFKTSLW

SEQ ID NO: 21 - protein, 282 aa, Homo sapiens
MERPSLRALLLGAAGLLLLLLPLSSSSSSDTCGPCEPASCPPLPPLGCLLGETRDACGCCPMCARGEGEPCGGGGAGRGYCAPGMECVKSRKRRKGKAGAAAGGPGVSGVCVCKSRYPVCGSDGTTYPSGCQLRAASQRAESRGEKAITQVSKGTCEQGPSIVTPPKDIWNVTGAQVYLSCEVIGIPTPVLIWNKVKRGHYGVQRTELLPGDRDNLAIQTRGGPEKHEVTGWVLVSPLSKEDAGEYECHASNSQGQASASAKITVVDALHEIPVKKGEGAEL

SEQ ID NO: 22 - protein, 279 aa, Homo sapiens
MERPSLRALLLGAAGLLLLLLPLSSSSSSDTCGPCEPASCPPLPPLGCLLGETRDACGCCPMCARGEGEPCGGGGAGRGYCAPGMECVKSRKRRKGKAGAAAGGPGVSGVCVCKSRYPVCGSDGTTYPSGCQLRAASQRAESRGEKAITQVSKGTCEQGPSIVTPPKDIWNVTGAQVYLSCEVIGIPTPVLIWNKVKRGHYGVQRTELLPGDRDNLAIQTRGGPEKHEVTGWVLVSPLSKEDAGEYECHASNSQGQASASAKITVVDALHEIPVKKGTQ

SEQ ID NO: 23 - protein, 398 aa, Homo sapiens
MEGAALLRVSVLCIWMSALFLGVGVRAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTMDYGKKWWTQAQAHDLVIKSLDKLKEVREFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 24 - protein, 414 aa, Homo sapiens
MRFKSHTVELRRPCSDMEGAALLRVSVLCIWMSALFLGVGVRAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTMDYGKKWWTQAQAHDLVIKSLDKLKEVREFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 25 - protein, 398 aa, Homo sapiens; identical to SEQ ID NO: 23

SEQ ID NO: 26 - protein, 380 aa, Homo sapiens
MEGAALLRVSVLCIWVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTMDYGKKWWTQAQAHDLVIKSLDKLKEVREFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 27 - protein, 487 aa, Homo sapiens
METVQLRNPPRRQLKKLDEDSLTKQPEEVFDVLEKLGEGSYGSVYKAIHKETGQIVAIKQVPVESDLQEIIKEISIMQQCDSPHVVKYYGSYFKNTDLWIVMEYCGAGSVSDIIRLRNKTLTEDEIATILQSTLKGLEYLHFMRKIHRDIKAGNILLNTEGHAKLADFGVAGQLTDTMAKRNTVIGTPFWMAPEVIQEIGYNCVADIWSLGITAIEMAEGKPPYADIHPMRAIFMIPTNPPPTFRKPELWSDNFTDFVKQCLVKSPEQRATATQLLQHPFVRSAKGVSILRDLINEAMDVKLKRQESQQREVDQDDEENSEEDEMDSGTMVRAVGDEMGTVRVASTMTDGANTMIEHDDTLPSQLGTMVINAEDEEEEGTMKRRDETMQPAKPSFLEYFEQKEKENQINSFGKSVPGPLKNSSDWKIPQDGDYEFLKSWTVEDLQKRLLALDPMMEQEIEEIRQKYQSKRQPILDAIEAKKRRQQNF

SEQ ID NO: 28 - protein, 323 aa, Homo sapiens
MEPRVRVEGWKVPTSRCRFLLARVLGYLVVMEAVLTEELDEEEQLLRRHRKEKKELQAKIQGMKNAVPKNDKKRRKQLTEDVAKLEKEMEQKHREELEQLKLTTKENKIDSVAVNISNLVLENQPPRISKAQKRREKKAALEKEREERIAEAEIENLTGARHMESEKLAQILAARQLEIKQIPSDGHCMYKAIEDQLKEKDCALTVVALRSQTAEYMQSHVEDFLPFLTNPNTGDMYTPEEFQKYCEDIVNTAAWGGQLELRALSHILQTPIEIIQADSPPIIVGEEYSKKPLILVYMRHAYGLGEHYNSVTRLVNIVTENCS

SEQ ID NO: 29 - protein, 688 aa, Homo sapiens
MADLEAVLADVSYLMAMEKSKATPAARASKRIVLPEPSIRSVMQKYLAERNEITFDKIFNQKIGFLLFKDFCLNEINEAVPQVKFYEEIKEYEKLDNEEDRLCRSRQIYDAYIMKELLSCSHPFSKQAVEHVQSHLSKKQVTSTLFQPYIEEICESLRGDIFQKFMESDKFTRFCQWKNVELNIHLTMNEFSVHRIIGRGGFGEVYGCRKADTGKMYAMKCLDKKRIKMKQGETLALNERIMLSLVSTGDCPFIVCMTYAFHTPDKLCFILDLMNGGDLHYHLSQHGVFSEKEMRFYATEIILGLEHMHNRFVVYRDLKPANILLDEHGHARISDLGLACDFSKKKPHASVGTHGYMAPEVLQKGTAYDSSADWFSLGCMLFKLLRGHSPFRQHKTKDKHEIDRMTLTVNVELPDTFSPELKSLLEGLLQRDVSKRLGCHGGGSQEVKEHSFFKGVDWQHVYLQKYPPPLIPPRGEVNAADAFDIGSFDEEDTKGIKLLDCDQELYKNFPLVISERWQQEVTETVYEAVNADTDKIEARKRAKNKQLGHEEDYALGKDCIMHGYMLKLGNPFLTQWQRRYFYLFPNRLEWRGEGESRQNLLTMEQILSVEETQIKDKKCILFRIKGGKQFVLQCESDPEFVQWKKELNETFKEAQRLLRRAPKFLNKPRSGTVELPKPSLCHRNSNGL

SEQ ID NO: 30 - protein, 443 aa, Homo sapiens
MLPCASCLPGSLLLWALLLLLLGSASPQDSEEPDSYTECTDGYEWDPDSQHCRDVNECLTIPEACKGEMKCINHYGGYLCLPRSAAVINDLHGEGPPPPVPPAQHPNPCPPGYEPDDQDSCVDVDECAQALHDCRPSQDCHNLPGSYQCTCPDGYRKIGPECVDIDECRYRYCQHRCVNLPGSFRCQCEPGFQLGPNNRSCVDVNECDMGAPCEQRCFNSYGTFLCRCHQGYELHRDGFSCSDIDECSYSSYLCQYRCINEPGRFSCHCPQGYQLLATRLCQDIDECESGAHQCSEAQTCVNFHGGYRCVDTNRCVEPYIQVSENRCLCPASNPLCREQPSSIVHRYMTITSERSVPADVFQIQATSVYPGAYNAFQIRAGNSQGDFYIRQINNVSAMLVLARPVTGPREYVLDLEMVTMNSLMSYRASSVLRLTVFVGAYTF

SEQ ID NO: 31 - protein, 425 aa, Homo sapiens
MGPRRLLLVAACFSLCGPLLSARTRARRPESKATNATLDPRSFLLRNPNDKYEPFWEDEEKNESGLTEYRLVSINKSSPLQKQLPAFISEDASGYLTSSWLTLFVPSVYTGVFVVSLPLNIMAIVVFILKMKVKKPAVVYMLHLATADVLFVSVLPFKISYYFSGSDWQFGSELCRFVTAAFYCNMYASILLMTVISIDRFLAVVYPMQSLSWRTLGRASFTCLAIWALAIAGVVPLLLKEQTIQVPGLNITTCHDVLNETLLEGYYAYYFSAFSAVFFFVPLIISTVCYVSIIRCLSSSAVANRSKKSRALFLSAAVFCIFIICFGPTNVLLIAHYSFLSHTSTTEAAYFAYLLCVCVSSISCCIDPLIYYYASSECQRYVYSILCCKESSDPSSYNSSGQLMASKMDTCSSNLNNSIYKKLLT

SEQ ID NO: 32 - protein, 581 aa, Homo sapiens
MPAPRAREQPRVPGERQPLLPRGARGPRRWRRAAGAAVLLVEMLERAAFFGVTANLVLYLNSTNFNWTGEQATRAALVFLGASYLLAPVGGWLADVYLGRYRAVALSLLLYLAASGLLPATAFPDGRSSFCGEMPASPLGPACPSAGCPRSSPSPYCAPVLYAGLLLLGLAASSVRSNLTSFGADQVMDLGRDATRRFFNWFYWSINLGAVLSLLVVAFIQQNISFLLGYSIPVGCVGLAFFIFLFATPVFITKPPMGSQVSSMLKLALQNCCPQLWQRHSARDRQCARVLADERSPQPGASPQEDIANFQVLVKILPVMVTLVPYWMVYFQMQSTYVLQGLHLHIPNIFPANPANISVALRAQGSSYTIPEAWLLLANVVVVLILVPLKDRLIDPLLLRCKLLPSALQKMALGMFFGFTSVIVAGVLEMERLHYIHHNETVSQQIGEVLYNAAPLSIWWQIPQYLLIGISEIFASIPGLEFAYSEAPRSMQGAIMGIFFCLSGVGSLLGSSLVALLSLPGGWLHCPKDFGNINNCRMDLYFFLLAGIQAVTALLFVWIAGRYERASQGPASHSRFSRDRG

SEQ ID NO: 33 - protein, 380 aa, Homo sapiens
MKKSIGILSPGVALGMAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 34 - protein, 365 aa, Homo sapiens
MAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 35 - DNA, 19 bp, Artificial sequence
  siRNA target sequence
gaatcgatattgttacaac

SEQ ID NO: 36 - DNA, 19 bp, Artificial sequence
  siRNA target sequence
atatcgaggtgaacatcac

SEQ ID NO: 37 - DNA, 19 bp, Artificial sequence
  siRNA target sequence
gcagtcaagtttccacaac

SEQ ID NO: 38 - DNA, 19 bp, Artificial sequence
  siRNA target sequence
gctccatctcctactacga

SEQ ID NO: 39 - DNA, 19 bp, Artificial sequence
  siRNA target sequence
gtgttccattgcttacttt

SEQ ID NO: 40 - DNA, 19 bp, Artificial sequence
  siRNA target sequence
gcagagtaatgctccatca

SEQ ID NO: 41 - DNA, 19 bp, Artificial sequence
  siRNA target sequence
gaaagcattggcaaaggtc

SEQ ID NO: 42 - DNA, 19 bp, Artificial sequence
  siRNA target sequence
gcagtcaagtttccacaac

SEQ ID NO: 43 - DNA, 19 bp, Artificial sequence
  siRNA target sequence
ctgtgtcacaatcacccac

SEQ ID NO: 44 - DNA, 19 bp, Artificial sequence
  siRNA target sequence
gaactgcgagatactgatt

SEQ ID NO: 45 - DNA, 19 bp, Artificial sequence
  siRNA target sequence
acagatgcctttctgtgac

SEQ ID NO: 46 - DNA, 19 bp, Artificial sequence
  siRNA target sequence
acttctgagaggtcacagc

SEQ ID NO: 47 - DNA, 19 bp, Artificial sequence
  siRNA target sequence
gaacacgtacaaagtcatt

SEQ ID NO: 48 - DNA, 19 bp, Artificial sequence
  siRNA target sequence
ggatggagttgggaatcac

SEQ ID NO: 49 - DNA, 19 bp, Artificial sequence
  siRNA target sequence
gaggatgccattaagtatt

SEQ ID NO: 50 - DNA, 19 bp, Artificial sequence
  siRNA target sequence
gaggcagccttgtactctt

SEQ ID NO: 51 - DNA, 19 bp, Artificial sequence
  siRNA target sequence
ggatcttgggtcctatccc

SEQ ID NO: 52 - DNA, 19 bp, Artificial sequence
  siRNA target sequence
tgaatactatgtgggattc

SEQ ID NO: 53 - DNA, 19 bp, Artificial sequence
  siRNA target sequence
tcagctgggcgctatgttc

SEQ ID NO: 54 - DNA, 19 bp, Artificial sequence
  siRNA target sequence
gactggaaagcgacgggtc

SEQ ID NO: 55 - DNA, 19 bp, Artificial sequence
  siRNA target sequence
aggctcacttgcctttggc

SEQ ID NO: 56 - DNA, 19 bp, Artificial sequence
  siRNA target sequence
tgatggttaccgcaagatc

SEQ ID NO: 57 - DNA, 19 bp, Artificial sequence
  siRNA target sequence
ccaaacctgtgtcaacttc

SEQ ID NO: 58 - DNA, 19 bp, Artificial sequence
  siRNA target sequence
gatcccagcagttataaca

SEQ ID NO: 59 - DNA, 19 bp, Artificial sequence
  siRNA target sequence
tgaaggtcaagaagccggc

SEQ ID NO: 60 - DNA, 19 bp, Artificial sequence
  siRNA target sequence
aacctggccattcagaccc

SEQ ID NO: 61 - DNA, 19 bp, Artificial sequence
  siRNA target sequence
caattcccaaggacaggct

SEQ ID NO: 62 - DNA, 19 bp, Artificial sequence
  siRNA target sequence
cagattccatctgatggcc

SEQ ID NO: 63 - DNA, 19 bp, Artificial sequence
  siRNA target sequence
gaatttcagaagtactgtg

SEQ ID NO: 64 - DNA, 19 bp, Artificial sequence
  siRNA target sequence
gtccaacagaagtacgtgc

SEQ ID NO: 65 - DNA, 19 bp, Artificial sequence
  siRNA target sequence
ggccatgattgagaaactc

SEQ ID NO: 66 - DNA, 19 bp, Artificial sequence
  siRNA target sequence
gaaggagctactcatcttc

SEQ ID NO: 67 - DNA, 19 bp, Artificial sequence
  siRNA target sequence
caagagcgatgcctattac

SEQ ID NO: 68 - DNA, 19 bp, Artificial sequence
  siRNA target sequence
catcagcttcctgctgggc

SEQ ID NO: 69 - DNA, 19 bp, Artificial sequence
  siRNA target sequence
gatggagcgcttacactac

SEQ ID NO: 70 - DNA, 19 bp, Artificial sequence
  siRNA target sequence
gagtttgcctactcagagg

SEQ ID NO: 71 - DNA, 19 bp, Artificial sequence
  siRNA target sequence
cacggctctcctatttgtc

SEQ ID NO: 72 - DNA, 19 bp, Artificial sequence
  siRNA target sequence
gagttggacagtggaggac

SEQ ID NO: 73 - DNA, 19 bp, Artificial sequence
  siRNA target sequence
gaaaccatcctttcttgaa

SEQ ID NO: 74 - DNA, 19 bp, Artificial sequence
  siRNA target sequence
agacctggtctacatcgac

SEQ ID NO: 75 - DNA, 19 bp, Artificial sequence
  siRNA target sequence
tcgctaggtatgaataacc

SEQ ID NO: 76 - RNA, 12 nt, Artificial sequence
  Loop sequence
guuugcuauaac

SEQ ID NO: 77 - DNA, 18 bp, Artificial sequence
  Forward primer
accctgtgctgctcaccg

SEQ ID NO: 78 - DNA, 23 bp, Artificial sequence
  Reverse primer
aggtctcaaacatgatctgggtc